SEQUENCE LISTING (28 sequences)

SEQ ID NO: 1 - protein, 232 aa, Homo sapiens [sequence omitted]

SEQ ID NO: 2 - protein, 181 aa, Homo sapiens
GPVPTPPDNIQVQENFNISRIYGKWYNLAIGSTCPLKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 3 - protein, 249 aa, Homo sapiens
RHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 4 - protein, 242 aa, Homo sapiens
RLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 5 - protein, 256 aa, Homo sapiens
RLAILPASAPPATSNPDPAVSRVMNMKIEETTMTTQTPACPSCSRSRAPAVPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 6 - protein, 300 aa, Homo sapiens
AKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 7 - protein, 399 aa, Homo sapiens
DEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPEVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - protein, 313 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 9 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 10 - protein, 291 aa, Homo sapiens
EAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 11 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 12 - protein, 348 aa, Homo sapiens
MSDLGAVISLLLWGRQLFALYSGNDVTDISDDRFPKPPEIANGYVEHLFRYQCKNYYRLRTEGDGVYTLNDKKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYHQVDIGLIKLKQKVLVNERVMPICLPSKNYAEVGRVGYVSGWGQSDNFKLTDHLKYVMLPVADQYDCITHYEGSTCPKWKAPKSPVGVQPILNEHTFCVGMSKYQEDTCYGDAGSAFAVHDLEEDTWYAAGILSFDKSCAVAEYGVYVKVTSIQDWVQKTIAEN

SEQ ID NO: 13 - protein, 347 aa, Homo sapiens
MALLFSLILAICTRPGFLASPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPESSFSPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSG

SEQ ID NO: 14 - protein, 347 aa, Homo sapiens
MALLFSLILAICTRPGFLASPSGVRLVGGLHRCEGRVEVEQKGQWGTVCDDGWDIKDVAVLCRELGCGAASGTPSGILYEPPAEKEQKVLIQSVSCTGTEDTLAQCEQEEVYDCSHDEDAGASCENPESSFSPVPEGVRLADGPGHCKGRVEVKHQNQWYTVCQTGWSLRAAKVVCRQLGCGRAVLTQKRCNKHAYGRKPIWLSQMSCSGREATLQDCPSGPWGKNTCNHDEDTWVECEDPFDLRLVGGDNLCSGRLEVLHKGVWGSVCDDNWGEKEDQVVCKQLGCGKSLSPSFRDRKCYGPGVGRIWLDNVRCSGEEQSLEQCQHRFWGFHDCTHQEDVAVICSV

SEQ ID NO: 15 - protein, 147 aa, Homo sapiens [sequence omitted]

SEQ ID NO: 16 - protein, 142 aa, Homo sapiens [sequence omitted]

SEQ ID NO: 17 - protein, 142 aa, Homo sapiens [sequence omitted]

SEQ ID NO: 18 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 19 - protein, 135 aa, Homo sapiens
DETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPR

SEQ ID NO: 20 - protein, 114 aa, Homo sapiens
AESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKK

SEQ ID NO: 21 - protein, 148 aa, Homo sapiens
AESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDR

SEQ ID NO: 22 - protein, 127 aa, Homo sapiens
AESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEK

SEQ ID NO: 23 - protein, 698 aa, Homo sapiens; identical to SEQ ID NO: 9

SEQ ID NO: 24 - protein, 351 aa, Homo sapiens
MRSLGALLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 25 - protein, 928 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMNIDGMTYPGIIKEKAEAQAQYSAAVAKGKNAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 26 - protein, 464 aa, Homo sapiens
QDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSAGAAGSRMNFRPGVLSSRQLGLPGPPDVPDHAAYHPFRRLAILPASAPPATSNPDPAVSRVMNMKIEETTMTTQTPACPSCSRSRAPAVPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 27 - protein, 480 aa, Homo sapiens
QDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 28 - protein, 1744 aa, Homo sapiens [sequence omitted]

(19) United States
(12) Patent Application Publication
(10) Pub. No.: US 2010/0004871 A1
(43) Pub. Date: Jan. 7, 2010

(54) Identities, specificities, and use of twenty two (22) differentially expressed protein biomarkers for blood based diagnosis of breast cancer

(75) Inventor: Ira L. Goldknopf, The Woodlands, TX (US)

Correspondence Address: Benjamin A. Adler, PhD, JD, 8011 Candle Lane, Houston, TX, 77071, US

(73) Assignee: Power3 Medical Products, Inc., The Woodlands, TX (US)

(21) Appl. No.: 12/313,136

(22) Filed: Nov. 17, 2008

Related U.S. Application Data
(63) Continuation-in-part of application No. 11/635,281, filed on Dec. 7, 2006.
(60) Provisional application No. 60/754,441, filed on Dec. 27, 2005.

(51) Int. Cl. G06F 19/00; C40B 40/10; C07K 14/00; C07K 16/00; C07K 14/775; C07K 14/76; C07K 14/52; G06F 17/18

(52) U.S. Cl. 702/19; 506/18; 530/350; 530/387.1; 530/359; 530/363; 530/351; 702/179

(57) ABSTRACT

The present invention discloses twenty two 22 protein biomarkers of breast cancer. More specifically, the present invention discloses the identities, specificities, and uses of up to twenty two (22) protein biomarkers in blood serum for distinguishing between patients with earlier and later stages of breast cancer, patients with benign breast diseases or abnormalities, and normal individuals lacking breast abnormalities.

### CROSS REFERENCE TO RELATED APPLICATIONS

This application claims priority under 35 U.S.C. §120 to pending nonprivisional U.S. Ser. No. 11/635,281, filed Dec. 7, 2006, which claims benefit of priority under 35 U.S.C. §119(e) of provisional U.S. Ser. No. 60/834,649, filed Aug. 1, 2006, now abandoned, and of provisional U.S. Ser. No. 60/754,441, filed Dec. 27, 2005, now abandoned.

### BACKGROUND OF THE INVENTION

1. Field of the Invention

This invention relates to twenty two (22) protein biomarkers of breast cancer. More specifically, the invention relates to the differential expression of up to 22 protein biomarkers in blood serum that can be used in diagnosis, determination of disease severity, and monitoring of therapeutic response of patients with breast cancer. The method is based on the use of two-dimensional (2D) gel electrophoresis to separate the complex mixture of proteins found in blood serum, the quantitation of up to 22 identified protein spots, and statistical analysis, to distinguish between patients with early and later stages of breast cancer, patients with benign breast disease or abnormalities, and normal women, for the purpose of screening, diagnosis, for determination of disease severity, and for treatment response monitoring.

2. Description of the Related Art

There is an urgent need for objective diagnostic tests to detect breast cancer in its earliest stages. By the time a patient is diagnosed with breast cancer by mammography and subsequent biopsy, the patient has had the disease for an average 6-10 years (Spratt, J. S. et al. 1986, Cancer Research 46, 970-974, A. Hollingsworth, personal communication Dec. 2, 2004 re Spratt et al). In addition, when mammography is the only screening tool utilized, it has to be remembered that sensitivity here is only 70% overall even with digital technology, and mammography was recently found in a major trial to have a mere 41% sensitivity when a 15-month follow-up period was used to define false-negatives. (Pisano et al. 2005, N Engl J Med 353, 1773-1783). MRI detects breast cancer earlier, and with much greater sensitivity, than mammograms (Hollingsworth, A. B. et al. 2003, J. OK. St. Med. Assoc. 96, Hollingsworth A. B. et al. 2004 Amer. J. Surgery 187 349-362). Genetic mutational tests (BRCA 1 and 2 genes) detect genetic disposition of breast cancer risk, but aggressive screening, usually with breast MRI, is chosen more often than preventive mastectomy by patients who tests BRCA-positive (Hollingsworth A. B. et al. 2004; Robson, M. E. et al. 2004, JAMA 292, 1368-1370). Whereas the need for imaging of breast tumors will always be required for localization and treatment, a sensitive early detection screening test with cost comparable to mammograms is needed to justify the high cost and insurance reimbursement for auxiliary imaging with ultrasound and/or MRI.

There has been a tremendous interest in the potential ability of proteomic technology to fulfill the unmet needs of effective strategies for early diagnosis of cancer (Alaiya, A. et al. 2005, J. Proteome Res. 4: 1213-1222) with a special emphasis on cancer detection in biological fluids from patients, including ovarian cancer (Emmanuel F. Petricoin, A. M. Ardekani, B. A. Hitt et al. 2002, Lancet 359: 572-577) and breast cancer (Paweletz C. P. et al 2001, Dis. Markers 17: 301-307; Henry M. Kuerer, H. M. et al. 2002, Cancer 95: 2276-2282). Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of proteins already known to be involved in certain biological functions has progressed from research applications alone to use in disease screening and diagnosis for clinicians. However, proteomic testing for diagnostic purposes remains in its infancy.

Detection of abnormalities in the genome of an individual can reveal the risk or potential risk for individuals to develop a disease. The transition from gene based risk to emergence of disease can be characterized as an expression of genomic abnormalities in the proteome. In fact, whether arising from genetic, environmental, or other factors, the appearance of abnormalities in the proteome signals the beginning of the process of cascading effects that can result in the deterioration of the health of the patient. Therefore, detection of proteomic abnormalities at an early stage is desired in order to allow for detection of disease processes either before the disease is established or in its earliest stages where treatment may be more effective.

Recent progress using a novel form of mass spectrometry called surface enhanced laser desorption and ionization time of flight (SELDI-TOF) for the testing of ovarian cancer and Alzheimer's disease has led to an increased interest in proteomics as a diagnostic tool (Petrocoin, E. F. et al. 2002. Lancet 359:572-577, Lewczuk, P. et al. 2004. Biol. Psychiatry 55:524530). Furthermore, proteomics has been applied to the study of breast cancer through use of 2D gel electrophoresis and image analysis to study the development and progression of breast carcinoma in patients' breast ductal fluid specimens (Kuerer, H. M. et al. 2002. Cancer 95:2276-2282) and in plasma (Goufman, et al. 2006. Biochemistry 2006, 71(4):35460). In the case of breast cancer, breast ductal fluid specimens were used to identify distinct protein expression patterns in bilateral matched pair ductal fluid samples of women with unilateral invasive breast carcinoma (Kuerer, H. M. et al. 2002).

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B1 discloses measurement of total urinary 17 ketosteroid-sulfates as biomarkers of biological age. U.S. Pat. No. 6,693,177 B1 discloses a process for preparation of a single biomarker specific for O-acetylated sialic acid and useful for diagnosis and outcome monitoring in patients with lymphoblastic leukemia.

Two-dimensional (2D) gel electrophoresis has been used in research laboratories for biomarker discovery since the 1970's (Margolis J. et al. 1969, Nature. 1969 221: 1056-1057; Orrick, L. R. et al. 1973; Proc Nat'l Acad. Sci. USA. 70: 1316-1320; Goldknopf, I. L. et al. 1975, J Biol Chem. 250: 7182-7187; Goldknopf, I. L. et al. 1977, Proc Nat'l Acad Sci USA. 74: 5492-5495; O'Farrell, P. H. 1975, J. Biol. Chem. 250: 4007-4021; Anderson, L. 1977, Proc Nat'l Aced Sci USA. 74: 864-868; Klose, J. 1975, Human Genetic. 26: 231-243). The advent of much faster identification of proteins spots by in-gel digestion and mass spectroscopy ushered in the accelerated development of proteomic science through large-scale application of these techniques (Aebersold R. 2003, Nature, 422: 198-207; Kuruma, H. et al. 2004, Prostate Cancer and Prostatic Disease 1: 1-8; Kuncewicz, T. et al. 2003, Molecular & Cellular Proteomics 2: 156-163). With the advent of bioinformatics, progression of proteomics towards diagnostics and personalized medicine has become feasible (White, C. N. et al. 2004 Clinical Biochemistry, 37: 636-641; Anderson N. L. et al. 2002, Molecular & Cellular Proteomics 1:845-867). Clinical proteomics is maturing fast into a powerful approach for comprehensive analyses of disease mechanisms and disease markers (Kuruma, H. et al. 2004; Sheta, E. A. et al. 2006, Expert Rev. Proteomics 3: 45-62). We have recently applied 2D gel proteomics of human serum combined with discriminant biostatistics to the differential diagnosis of neurodegenerative diseases (Goldknopf, I. L. et al. 2006, Biochem. Biophys. Res. Commun. 342: 1034-1039; Sheta, E. A. et al. 2006). In the present invention, we use the same approach to monitor the concentrations of 22 protein biomarkers, resolved and quantitated by 2D gel electrophoresis of blood serum, to distinguish between patients who have been diagnosed with earlier and later stages of breast cancer, with benign breast disease, and with no breast abnormalities as normal controls.

### SUMMARY OF THE INVENTION

The present invention relates to 22 protein biomarkers in blood serum for screening, diagnosis, determination of disease severity, and monitoring response to treatment, of breast cancer. More specifically, the present invention consists of up to 22 protein biomarkers in blood and their use in diagnostic assays for differentiating between patients with earlier and later stages of breast cancer, patients having benign breast disease or abnormalities, and normal individuals. The method comprises collecting a biological sample from patients having biopsy confirmed and histological staged breast cancer, patients having benign breast disease or abnormalities, and patients having no evidence of breast disease or breast abnormality, then determining the concentrations of up to 22 protein biomarkers identified as related to breast cancer. Patients are then sorted into these respective groupings based on a statistical analysis of the concentration in blood serum of up to 22 protein biomarkers.

One aspect of the present invention is the use of up to 22 biomarkers for screening a patient for breast cancer. The method includes: collecting a biological sample from a patient, determining the concentrations of up to 22 protein biomarkers identified as related to breast cancer, and determining whether or not the patient has breast cancer, based on a statistical analysis of the concentration in blood serum of one or more of the selected 22 protein biomarkers. This aspect of the invention can be used as an early blood screen in patients to complement mammography, such that a negative mammogram but a positive blood test would signal the need for more sensitive imaging such as breast MRI. In the case of an equivocal mammogram, the predictive power of a blood test would help the radiologist to decide whether or not to proceed with biopsy.

Another aspect of the present invention is the use of up to 22 protein biomarkers for determining the severity of breast cancer and/or monitoring the response to treatment of a patient. The method includes: collecting a biological sample from a patient, determining the concentrations of up to 22 protein biomarkers identified as related to breast cancer, and determining the severity of breast cancer and/or response of the patient to treatment based on the concentrations in blood serum of up to 22 protein biomarkers. For example, this aspect of the invention can be used to help the oncologist make decisions about specific chemotherapeutic and/or anti-hormonal regimens, and/or therapeutic antibodies and/or other therapeutic agents and regimens, and to monitor the response of the patient to treatment.

Another aspect of the present invention is the use of up to 22 biomarkers for determining the biological mechanism of disease of a patient and/or the drug target of the patient for treatment of breast cancer. The method includes: collecting a biological sample from a patient, determining the concentrations of up to 22 protein biomarkers identified as related to breast cancer, and determining the mechanism of disease active in the patient and/or identifying the drug target appropriate for treatment of the patient, based on the concentration in blood serum of up to 22 protein biomarkers.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiments disclosed might be readily utilized as a basis for modifying or redesigning the methods for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1: Representative 2D gel electrophoretic image of human serum proteins with the positions of 4 of the 22 protein biomarker spots, the electrophoretic isoforms of the Inter-alpha-trypsin inhibitor heavy chain (H4) related 35 KD (ITI (H4) RP 35 KD) protein spots B2422, B2505, B3410, and B4404, indicated by arrows, circles and numbers.

FIGS. 2A-2D: Statistical box and whiskers plots (constructed using Analyze-it software for Microsoft XL) of blood serum concentrations (2D gel spot density, PPM) of the four electrophoretic isoforms of the Inter-alpha-trypsin inhibitor heavy chain (H4) related 35 KD (ITI (H4) RP 35 KD) protein spots: FIG. 2A: B2422; FIG. 2B: B2505; FIG. 2C: B3410; and FIG. 2D: B4404, as depicted in FIG. 1, from patients with breast cancer (BC), benign breast abnormalities or disease (B9), and normal controls subjects (N). B2505 is up-regulated in breast cancer and B2422, B3410 and B4404 are down-regulated in breast cancer. Summary statistics are illustrated in Table XXXIII a-d.

FIGS. 3A-3D: Statistical box and whiskers plots and Receiver Operator Characteristics (ROC) plot (constructed using Analyze-it software for Microsoft XL) of blood serum concentrations of the sum of the four electrophoretic isoforms of the biomarker Inter-α-Trypsin Heavy Chain Related (H4) Protein, 35 KD, processing product (ITI (H4) RP 35 KD), corresponding to the sum of biomarker spots (B2422+B2505+B3410+B4404) in normal control subjects (N), patients with benign breast abnormalities or disease (B9), and breast cancer patients (BC), expressed both as: FIG. 3A, FIG. 3B: concentration=2D gel spot density (PPM); and as FIG. 3C, FIG. 3D: differential expression from normal=fold of average normal 2D gel spot density (PPM) (i.e. Normalized to the average of the normal concentrations). Values for retrospective and prospective samples determined separately and then combined for statistical analysis. Summary statistics are depicted: for FIGS. 3A, 3B in Table XXXIII e and Table XXXIV a; and for FIGS. 3C, 3D in Table XXXIV b.

FIGS. 4A-4D Statistical box and whiskers plots of the differential expression from normal of the blood serum concentrations (as fold of average normal blood serum concentration (2D gel spot density PPM) of the four electrophoretic isoforms of Inter-α-Trypsin Heavy Chain (H4) Related 35 KD protein spots: FIG. 4A: B2422; FIG. 4B: B2505; FIG. 4C: B3410; and FIG. 4D: B4404, in normal control subjects (N), patients with benign breast abnormalities or disease (B9), combined non-breast cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC). Summary statistics are depicted in Table XXXV.

FIGS. 5A-5D: Receiver Operator Characteristics (ROC) of the patients in FIG. 4, including sensitivities and specificities of diagnosis based on the individual performances of the four electrophoretic isoforms of Inter-α-Trypsin Heavy Chain (H4) Related 35 KD protein spots: FIG. 5A: B2422; FIG. 5B: B2505; FIG. 5C: B3410; and FIG. 5D: B4404.

FIG. 6: A representative 2D gel electrophoretic image of human serum proteins with the positions of the 22 protein biomarker spots: B1322; B1418; B2317; B2422; B2505; B3406; B3410; B4404; B5539; B6519; B6605; B7408; B1512; B2412; B4008; B4206; B3506; B4414; B5713; B6014; B6218; and B7108, indicated by arrows, circles and numbers.

FIGS. 7A-7B: illustrates: FIG. 7A: the estimation of the molecular weights (MW) of protein biomarker spots: B1322; B1418; B2317; B2422; B2505; B3406; B3410; B4404; B5539; B6519; B6605; B7408; B1512; B2412; B4008; B4206; B3506; B4414; B5713; B6014; B6218; and B7108, by 2D gel electrophoresis (relative migration in the SDS second dimension) employing protein standards of known molecular weights; and FIG. 7B: the estimation of isoelectric points of protein biomarker spots: B1322; B1418; B2317; B2422; B2505; B3406; B3410; B4404; B5539; B6519; B6605; B7408; B1512; B2412; B4008; B4206; B3506; B4414; B5713; B6014; B6218; and B7108, by 2D gel electrophoresis (relative focusing position in the isoelectric focusing first dimension between the extremes of the pH gradient, pH 5-8). Summary data are depicted in Tables III-V.

FIGS. 8A-8B: FIG. 8A: Statistical box and whiskers plots of the differential expression from normal of the blood serum concentrations (as fold of average normal blood serum concentration (2D gel spot density, PPM) of immunoglobulin lambda (λ) light chain spot B1322, in normal control subjects, patients with benign breast abnormalities, combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 8B: Receiver Operator Characteristics (ROC) of immunoglobulin lambda (λ) light chain spot B1322 for the patients in FIG. 8, including sensitivities and specificities of diagnosis for differentiation between N vs. B9; N vs. Non-DCIS BC; and N vs. DCIS BC. Summary statistics are depicted in Table XXXVI.

FIGS. 9A-9B: FIG. 9A: Statistical box and whiskers plots of the differential expression from normal of the blood serum concentrations (as fold of average normal blood serum concentration (2D gel spot density, PPM) of alpha-1-microglobulin protein spot B1418 in normal control subjects, patients with benign breast abnormalities, combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 9B: Receiver Operator Characteristics (ROC) of alpha-1-microglobulin protein spot B1418 with sensitivities and specificities of diagnosis for differentiation of N vs. DCIS BC; N vs. Non-DCIS BC; and N vs. combined BC. Summary statistics are depicted in Table XXXVII.

FIGS. 10A-10B: FIG. 10A: Statistical box and whiskers plots of the differential expression from normal of the blood serum concentrations (as fold of average normal blood serum concentration (fold of 2D gel spot density, PPM) of Apolipoprotein A-I protein spot B2317, in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 10B: Receiver Operator Characteristics (ROC) of Apolipoprotein A-I protein spot B2317 with sensitivities and specificities of diagnosis for distinguishing DCIS BC vs. N; DCIS BC vs. B9; B9 vs. N; Non-DCIA BC vs. B9; Non-DCIS BC vs. N; and Combined BC vs. N+B9. Summary statistics are depicted in Table XXXVIII.

FIGS. 11A-11B: FIG. 11A: Statistical box and whiskers plots of the differential expression from normal of the blood serum concentrations (as fold of average normal blood serum concentration (concentration=2D gel spot density, PPM) of Apolipoprotein E3 protein spot B3406 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 11B: Receiver Operator Characteristics (ROC) of Apolipoprotein E3 protein spot B3406 with sensitivities and specificities of diagnosis for distinguishing DCIS BC vs. N+B9. Summary statistics are depicted in Table XXXIX.

FIGS. 12A-12B: FIG. 12A: Statistical box and whiskers plots of the differential expression from normal of the blood serum concentrations (as fold of average normal blood serum concentration (concentration=normal 2D gel spot density, PPM) of Serum Albumin protein spot B5539 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 12B: Receiver Operator Characteristics (ROC) of Serum Albumin protein spot B5539, with sensitivities and specificities of diagnosis for distinguishing Non-DCIS BC vs. N+B9. Summary statistics are depicted in Table XL.

FIGS. 13A-13B: FIG. 13A: Statistical box and whiskers plots of the differential expression from normal of the blood serum concentrations (as fold of average normal blood serum concentration (concentration=2D gel spot density, PPM) of Lectin P35 protein spot B6519 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 13B: Receiver Operator Characteristics (ROC) of Lectin P35 protein spot B6519, with sensitivities and specificities of diagnosis for distinguishing Combined BC vs. N. Summary statistics are depicted in Table XLIX.

FIGS. 14A-14B: FIG. 14A: Statistical box and whiskers plots of the differential expression from normal of the blood serum concentrations (as fold of average normal blood serum concentration (concentration=2D gel spot density, PPM) of Transferrin protein spot B6605 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 14B: Receiver Operator Characteristics (ROC) of Transferrin protein spot B6605, with sensitivities and specificities of diagnosis for distinguishing B9 vs. N and DCIS BC vs. N. Summary statistics are depicted in Table XLI.

FIGS. 15A-15B: FIG. 15A: Statistical box and whiskers plots of the differential expression from normal of the blood serum concentrations as fold of average normal blood serum concentration (concentration=2D gel spot density, PPM) of Complement C4A protein spot B7408 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 15B: Receiver Operator Characteristics (ROC) of Complement C4A protein spot B7408, with sensitivities and specificities of diagnosis for distinguishing DCIS BC vs. N, B9 vs. N, and for not distinguishing Non-DCIS BC vs. N. Summary statistics are depicted in Table L.

FIGS. 16A-16D: FIG. 16A: Statistical box and whiskers plots of the differential expression from normal of the blood serum concentrations as fold of average normal blood serum concentration (concentration=2D gel spot density, PPM) of Haptoglobin protein spot B1512 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 16B-16D: Receiver Operator Characteristics (ROC) of Haptoglobin protein spot B1512, with sensitivities and specificities of diagnosis for distinguishing Non-DCIS BC vs. N, DCIS BC vs. B9 vs. N, vs. N+B9 vs. Combined BC. Summary statistics are depicted in Table XLIII.

FIG. 17: FIG. 17A: Statistical box and whiskers plots of the differential expression from normal of the blood serum concentrations (as fold of average normal blood serum concentration (2D gel spot density, PPM) of Apoptosis Inhibitor CD5L protein spot B2412 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 17B: Receiver Operator Characteristics (ROC) of Apoptosis Inhibitor CD5L protein spot B2412, with sensitivities and specificities of diagnosis for distinguishing Combined BC vs. N+B9. Summary statistics are depicted in Table LI.

FIGS. 18A-18B: FIG. 18A: Statistical box and whiskers plots of the differential expression from normal of the blood serum concentrations (as fold of average normal blood serum concentration (2D gel spot density, PPM) of Haptoglobin protein spot B4008 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 18B: Receiver Operator Characteristics (ROC) of Haptoglobin protein spot B4008, with sensitivities and specificities of diagnosis for distinguishing Combined BC vs. N. Summary statistics are depicted in Table XLV.

FIGS. 19A-19B: FIG. 19A: Statistical box and whiskers plots of the differential expression from normal of the blood serum concentrations as fold of average normal blood serum concentration (concentration=2D gel spot density, PPM) of Haptoglobin protein spot B4206 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 19B: Receiver Operator Characteristics (ROC) of Haptoglobin protein spot B4206, with sensitivities and specificities of diagnosis for distinguishing DCIS BC vs. N. Summary statistics are depicted in Table XLVI.

FIGS. 20A-20B: FIG. 20A: Statistical box and whiskers plots of the differential expression from normal of the blood serum concentrations as fold of average normal blood serum concentration (concentration=2D gel spot density, PPM) of Haptoglobin Related Protein spot B4424 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 20B: Receiver Operator Characteristics (ROC) of Haptoglobin Related Protein spot B4424, with lack of sensitivity and specificity of diagnosis for distinguishing N+B9 vs. BC. Summary statistics are depicted in Table XLVIII.

FIGS. 21A-21B: FIG. 21A: Statistical box and whiskers plots of the differential expression from normal of the blood serum concentrations as fold of average normal blood serum concentration (concentration=2D gel spot density, PPM) of Haptoglobin Related Protein spot B3506 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 21B: Receiver Operator Characteristics (ROC) of Haptoglobin Related Protein spot B3506, with lack of sensitivity and specificity of diagnosis for distinguishing N+B9 vs. Combined BC. Summary statistics are depicted in Table XLVII.

FIGS. 22A-22B: FIG. 22A: Statistical box and whiskers plots of the differential expression from normal of the blood serum concentrations as fold of average normal blood serum concentration (concentration=2D gel spot density, PPM) of Serotransferrin protein spot B5713 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 22B: Receiver Operator Characteristics (ROC) of Serotransferrin protein spot B5713, with sensitivities and specificities of diagnosis for distinguishing Combined BC vs. Normal. Summary statistics are depicted in Table XLII.

FIGS. 23A-23C: FIG. 23A: Statistical box and whiskers plots of the differential expression from normal of the blood serum concentrations as fold of average normal blood serum concentration (concentration=2D gel spot density, PPM) of Haptoglobin protein spot B6014 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC) wherein FIG. 23B: 62.2% of the Non-DCIS BC patients have detectable levels of Haptoglobin protein spot B6014 as compared to 32.3% of N+B9 patients and 33.3% of DCIS BC patients, and FIG. 23C: Receiver Operator Characteristics (ROC) with sensitivities and specificities of diagnosis for distinguishing Non-DCIS BC vs. N+B9 and Non-DCIS BC vs. DCIS BC based on detection of Haptoglobin protein spot B6014. Summary statistics are depicted in Table XLIV.

FIGS. 24A-24B: FIG. 24A: Statistical box and whiskers plots of the differential expression from normal of the blood serum concentrations as fold of average normal blood serum concentration (concentration=2D gel spot density, PPM) of Ribosomal and Nucleolar protein L27a protein spot B6218 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and FIG. 24B: Receiver Operator Characteristics (ROC) of Ribosomal and Nucleolar protein L27a spot B6218, with sensitivities and specificities of diagnosis for distinguishing DCIS BC vs. N+B9 and Non-DCIS BC vs. N+B9. Summary statistics are depicted in Table LII.

FIGS. 25A-25D): FIG. 25A: Statistical box and whiskers plots of the differential expression from normal of the blood serum concentrations as fold of average normal blood serum concentration (concentration=2D gel spot density, PPM) of NSB protein spot B7108 in normal control subjects (N), patients with benign breast abnormalities (B9), combined non-cancer controls (N+B9), combined breast cancer patients (DCIS BC+Non-DCIS BC); “purely invasive” breast cancer without in-situ breast cancer (Non-DCIS BC) and breast cancer patients with in-situ breast cancer (DCIS BC); and

FIGS. 25B-25D: Receiver Operator Characteristics (ROC) of NSB protein spot B7108, with sensitivities and specificities of diagnosis for distinguishing N vs. B9 vs. N+B9 vs. Combined BC vs. DCIS BC vs. Non-DCIS BC. Summary statistics are depicted in Table LIII.

FIGS. 26A-26B: Median differential expression profiles of blood serum concentrations of: FIG. 26A: the 22 breast cancer biomarkers; and FIG. 26B: 4 isoforms of the ITI (H4) RP 35 KD protein (protein spots B2505, B2422, B4404, B3410); 4 isoforms of a Haptoglobin protein (protein spots B6014, B1512; B4008, B4206); and 2 isoforms of a Haptoglobin related protein (B3506, B4424), as median fold of average mean spot concentration (concentration=2D gel spot density, PPM) for N, B9, DCIS-BC and Non-DCIS BC. Summary statistics are depicted in Table LIV.

Table I: Staging of Breast Cancer

Table II: Isoelectric points (pI) and molecular weights (Da) of standard protein mixture with isoforms separated as spots on 2D gels.

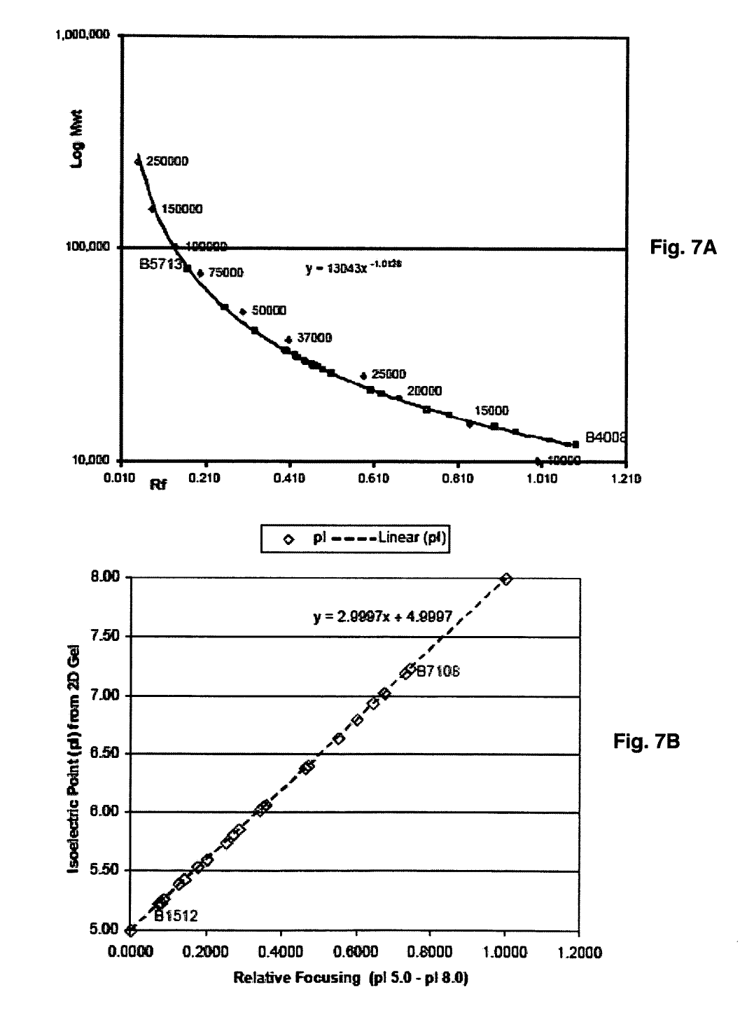

Table III: Molecular weights (MW) of the 22 breast cancer biomarker protein spots, based upon migration relative to the 10 KD protein standard in the SDS 2^{nd} dimension of the 2D gel electrophoresis as depicted in FIG. 7A.

Table IV: Isoelectric points (pI) of the 22 breast cancer biomarker protein spots, based upon their relative mobility, i.e. their position between the pH 5.0 and pH 8.0 range attained by isoelectric focusing in the 1^{st} dimension of the 2D gel electrophoresis as depicted in FIG. 7B.

Table V: Protein biomarker spot molecular weights (MW) and isoelectric points (pI) as determined from 2D gels (FIG. 7) as compared to the values calculated from the amino acid sequences as identified by LC MS/MS of the in-gel tryptic digests of the spots (Tables VI-XXXII, SEQ ID NOS: 1-22).

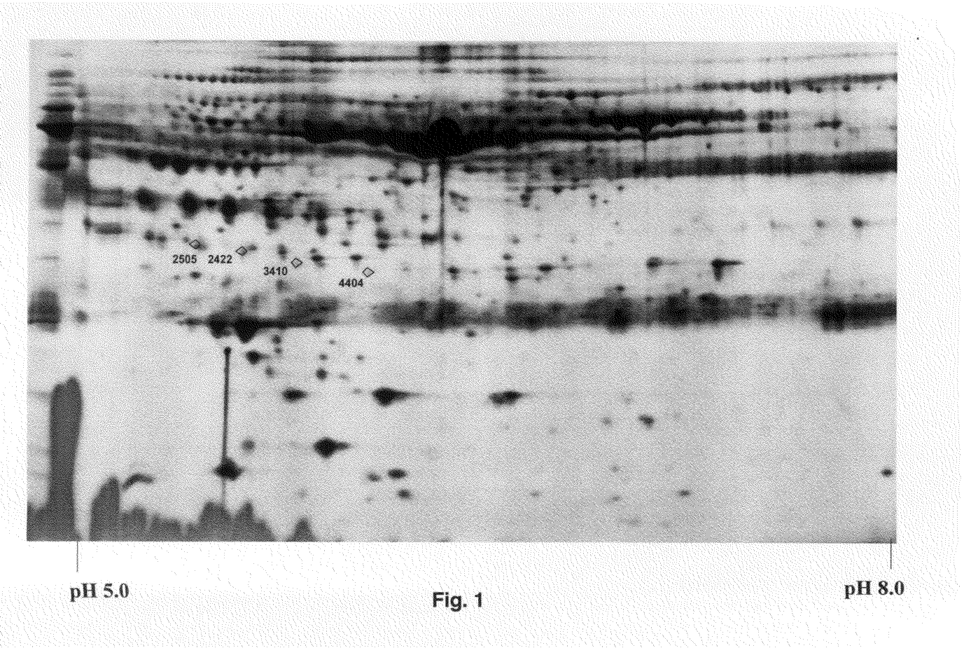

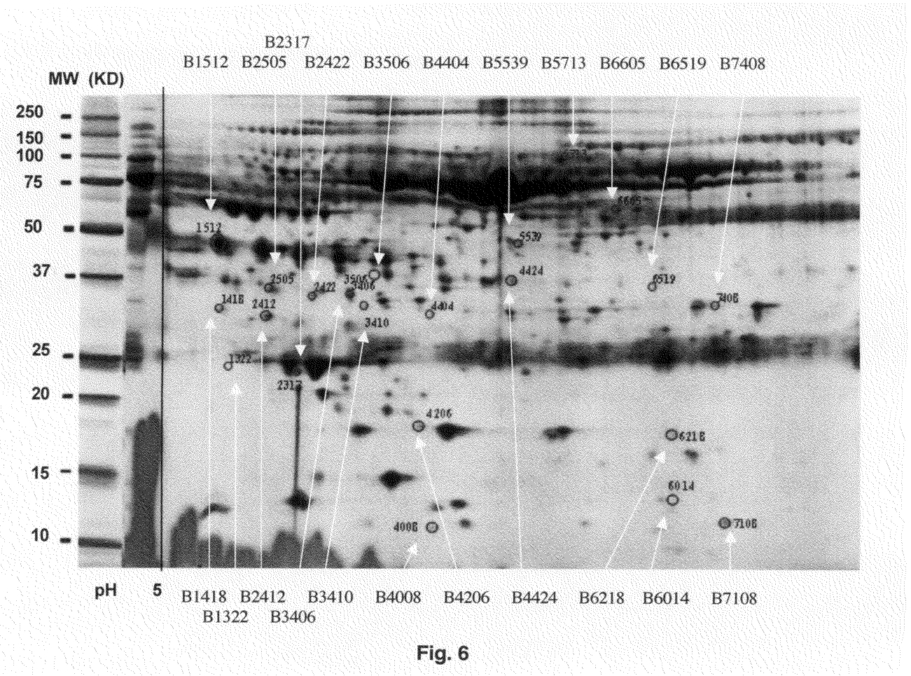

Table VI: The 22 Breast Cancer Biomarkers—Protein Identification by LC MSMS of 2D gel spot in-gel trypsin digests (FIGS. 1, 6, Tables VI-XXXII, SEQ ID NOS: 1-22).

Table VII: Single letter amino acid sequence (SEQ ID NO: 1) of Immunoglobulin Lambda Chain protein spot B1322.

Table VIII: Single letter amino acid sequence (SEQ ID NO:_{—}2) of Alpha-1-microglobulin protein spot B1418. Also shown is its placement in the single letter amino acid sequence of the precursor, which also contains the protein bikunin (SEQ ID NO: 24).

Table IX: Single letter amino acid sequence (SEQ ID NO: 3) of Apolipoprotein A-I protein spot B2317.

Table X: Amino acid sequence of Inter-alpha-Trypsin inhibitor heavy chain (H4) related protein (ITIHRP, PK120), the precursor to the 35 KD biomarker protein spots B2422, B2505, B3410, and B4404 (SEQ ID NO: 25). The placement of the Inter-alpha-trypsin Inhibitor Heavy Chain (H4) Related 35 KD Protein, and the corresponding 75 KD protein, are indicated within the sequence of the PK120 precursor.

Table XI: Single letter amino acid sequences of isoforms 1 (SEQ ID NO: 4) and 2 (SEQ ID NO: 5) of the Inter-alpha-trypsin Inhibitor Heavy Chain (H4) Related 35 KD protein spots B2422, B2505, B3410, and B4404.

Table XII: Single letter amino acid sequence alignment of the Inter-alpha-trypsin Inhibitor Heavy Chain (H4) Related 35 KD Protein Isoform 1 (SEQ ID NO: 26) and Isoform 2 (SEQ ID NO: 27). Identical sequences are marked with stars while unmatched sequences are marked by dashes.

Table XIII: Single letter amino acid sequence (SEQ ID NO: 6) of Apolipoprotein E3 protein spot B3406.

Table XIV: Single letter amino acid sequence (SEQ ID NO: 7) of human albumin protein spot B5539.

Table XV: Single letter amino acid sequence (SEQ ID NO: 8) of human Lectin P35 3 protein spot B6519.

Table XVI: Single letter amino acid sequence (SEQ ID NO: 9) of Transferrin protein spot B6605.

Table XVII: Single letter amino acid sequence (SEQ ID NO: 10) of Complement C4A gamma protein spot B7408.

Table XVIII: Single letter amino acid sequence of parental protein Complement C4A (SEQ ID NO: 28).

Table XIX: Single letter amino acid sequence (SEQ ID NO: 11) of Haptoglobin protein spots B1512; B4008; B4206; and B6014.

Table XX: Single letter amino acid sequence (SEQ ID NO: 12) of Haptoglobin-related protein spots B3506 and B4424.

Table XXI: Single letter amino acid sequences of peptides identified by LC MS/MS of in-gel tryptic digests of protein spot B2412.

Table XXII: Single letter amino acid sequence (SEQ ID NO: 13) of AIM protein spot B2412.

Table XXIII: Single letter amino acid sequence (SEQ ID NO: 14) of CD5L protein alternate sequence of protein spot B2412.

Table XXIV: Single letter amino acid sequence (SEQ ID NO: 23) of Serotransferrin protein B5713.

Table XXV: Single letter amino acid sequence (SEQ ID NO: 15) of nucleolar/ribosomal protein L27a protein spot B6218.

Table XXVI: Alternate single letter amino acid sequence (SEQ ID NO:_{—}16) of nucleolar/ribosomal protein L27a protein spot B6218.

Table XXVII: Alternate single letter amino acid sequence (SEQ ID NO: 17) of nucleolar/ribosomal protein L27a protein spot B6218.

Table XXVIII: Single letter amino acid sequence (SEQ ID NO: 18) of Reticulon-4 precursor to protein spot B7108.

Table XXIX: Single letter amino acid sequence (SEQ ID NO: 19) of Reticulon-4 protein spot B7108.

Table XXX: Alternate single letter amino acid sequence (SEQ ID NO: 20) of Reticulon-4 protein spot B7108.

Table XXXI: Alternate single letter amino acid sequence (SEQ ID NO: 21) of Reticulon-4 protein spot B7108.

Table XXXII: Alternate single letter amino acid sequence (SEQ ID NO: 22) of Reticulon-4 protein spot B7108.

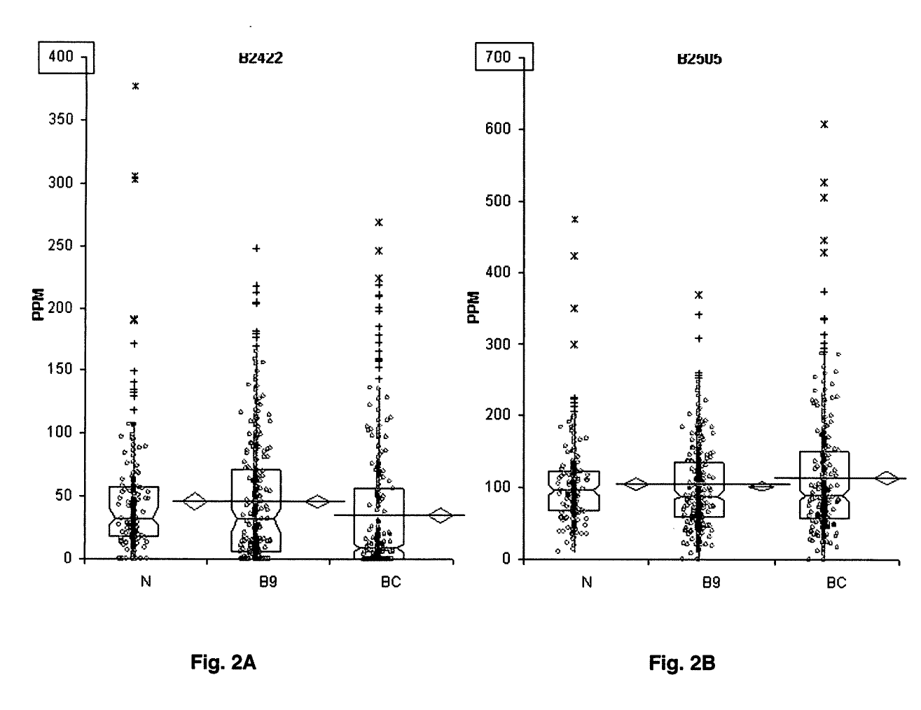

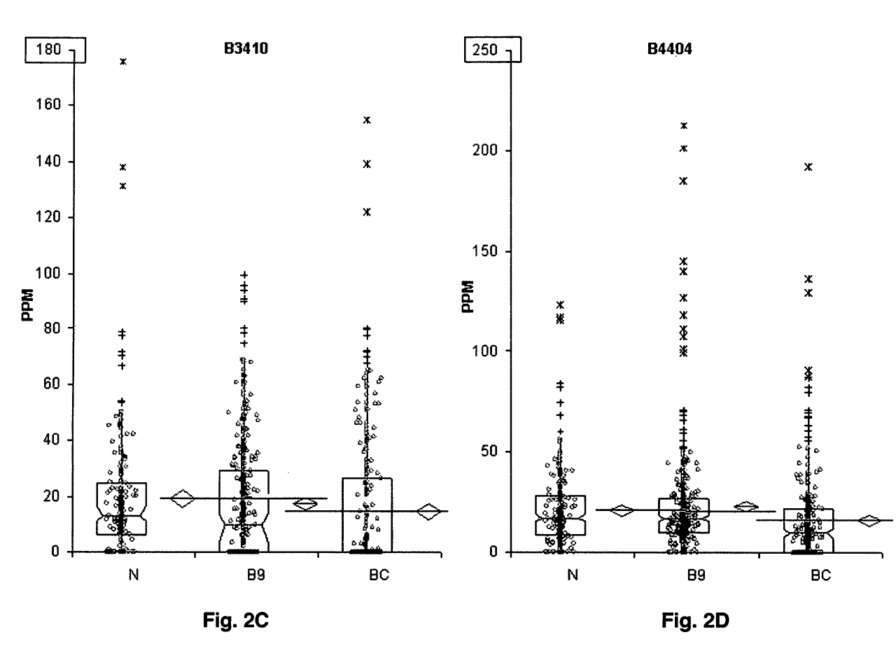

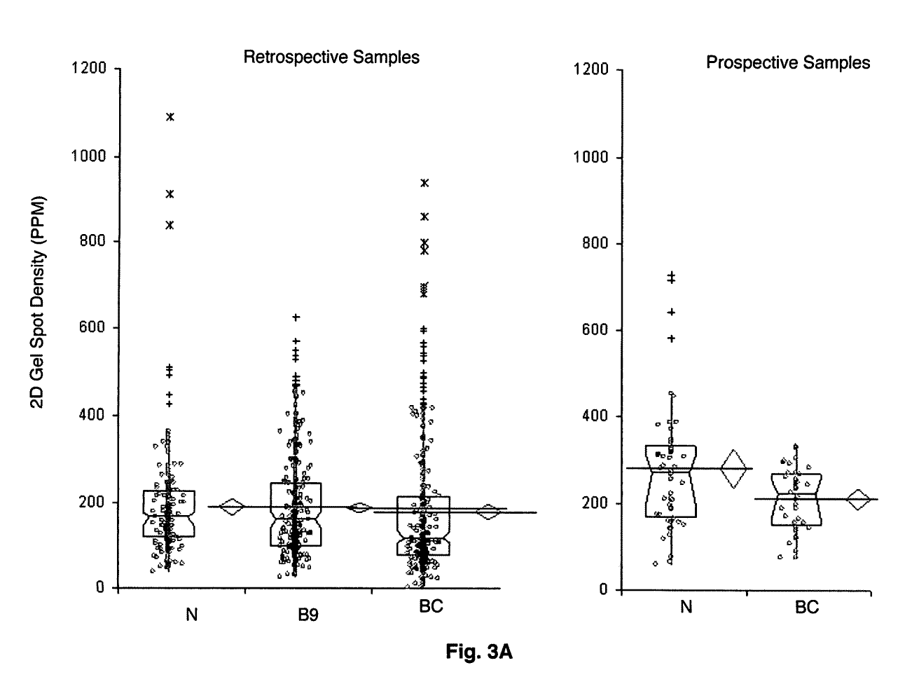

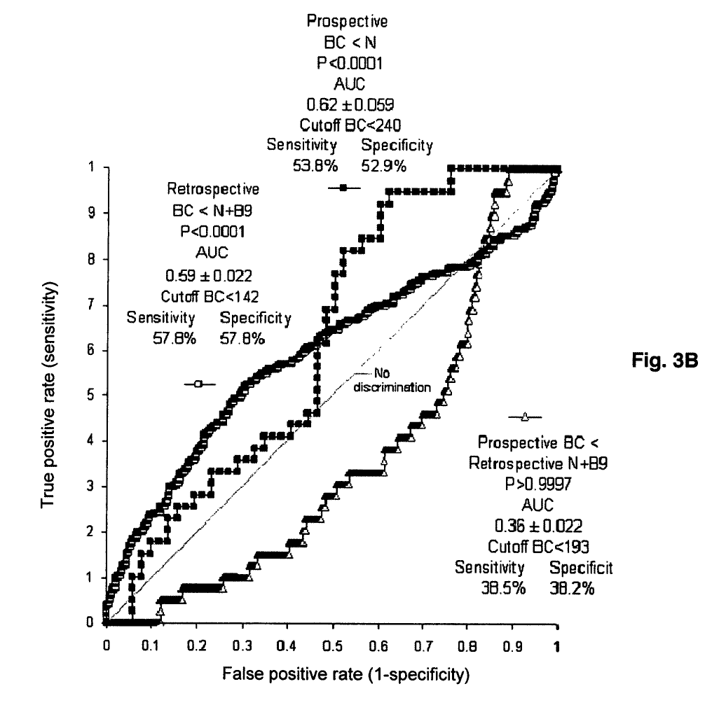

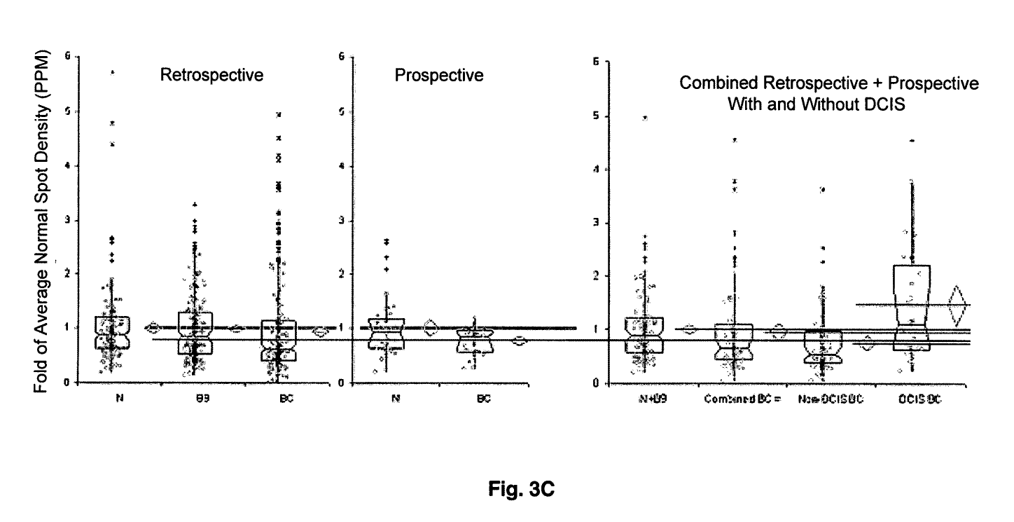

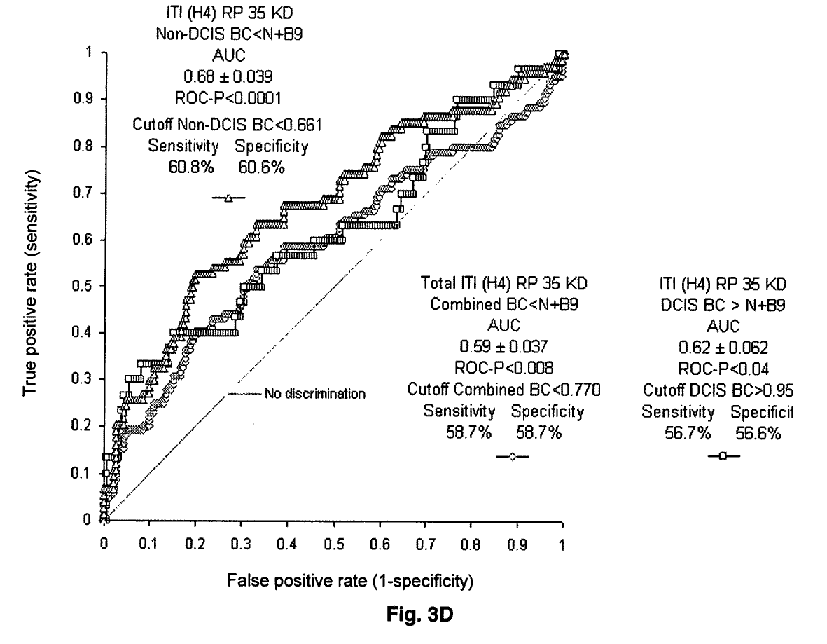

Table XXXIII: Summary statistics for ITI (H4) RP 35 KD isoform electrophoretic variants (FIG. 1) as depicted in graphs in FIG. 2, and for the sum of the isoforms (FIG. 3A, graph, retrospective samples, N, B9, BC).

Table XXXIV: Summary statistics for the Total ITI (H4) RP 35 KD proteins equal to the sum of the blood serum concentrations of protein spots B2422+B2505+B3410+B4404: a. measured as 2D gel spot density (PPM) as depicted in FIG. 3A; b measured as differential expression from normal as depicted in FIG. 3B, wherein differential expression from normal=fold of average normal concentration, and wherein concentration=2D gel spot density, PPM.

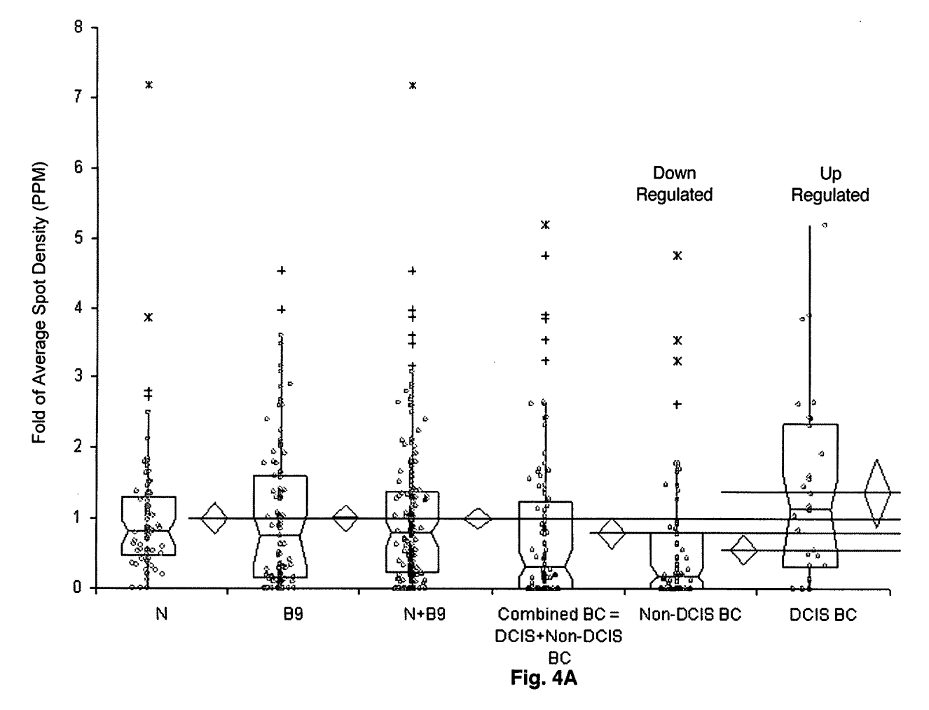

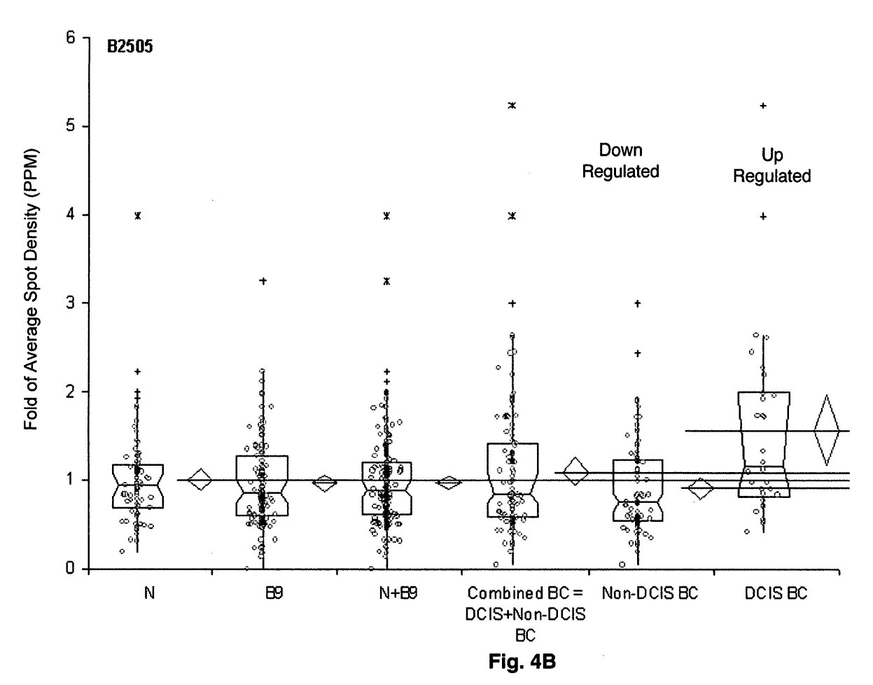

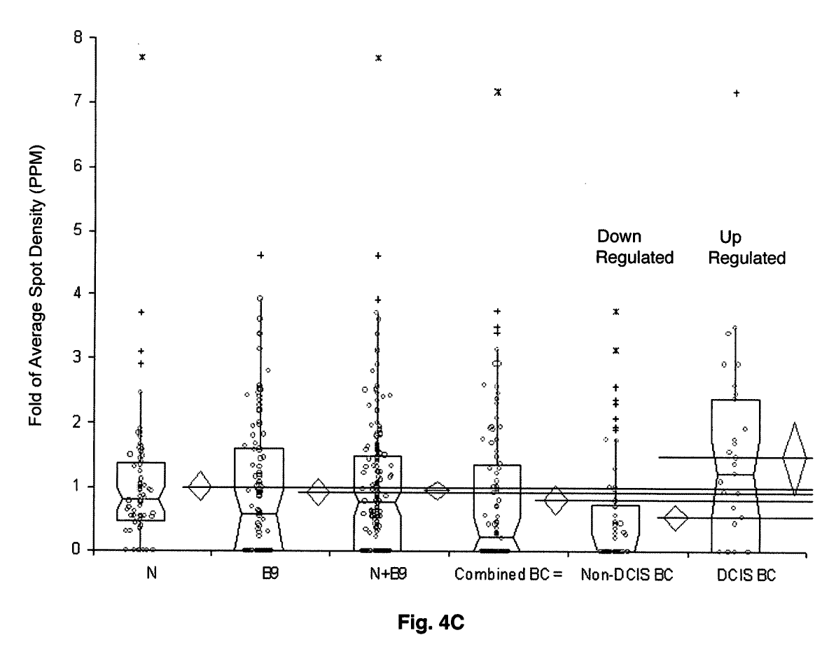

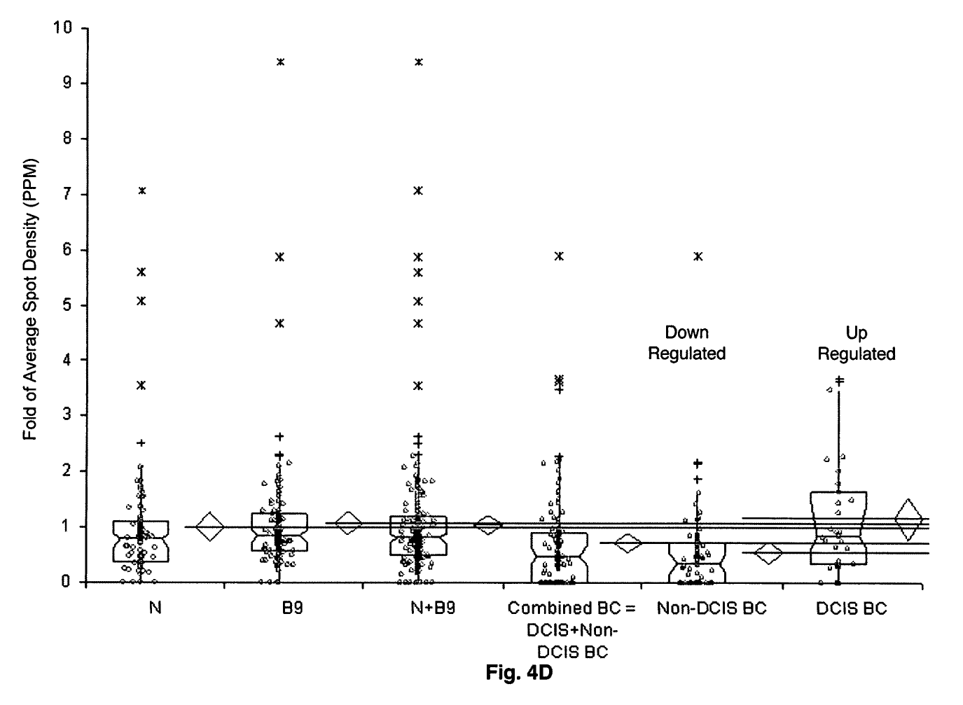

Table XXXV: Summary statistics of the differential expression of the Individual ITI (H4) RP 35 KD Protein Spots B2422, B2505, B3410, and B4404, equal to the fold of average normal blood serum concentration (concentration measured as protein spot density, PPM) as depicted in the graphs in FIGS. 4A-4D.

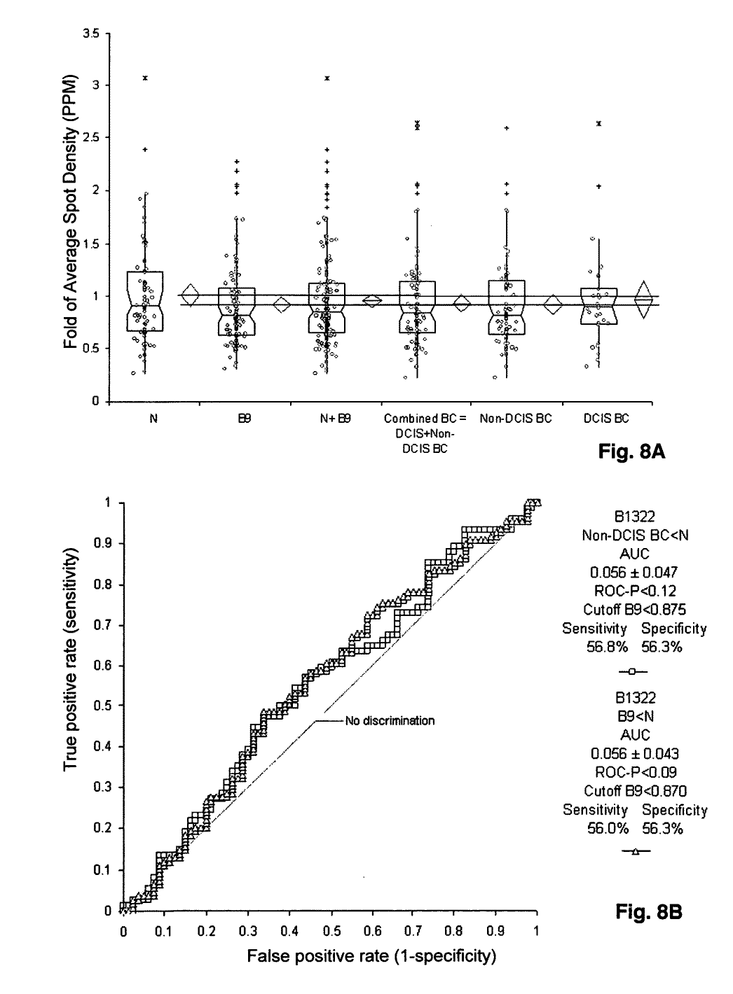

Table XXXVI: Summary statistics of the differential expression of Immunoglobulin lambda chain protein spot B1322, equal to the fold of average normal blood serum concentration (concentration measured as protein spot density, PPM) as depicted in the graph in FIG. 8A.

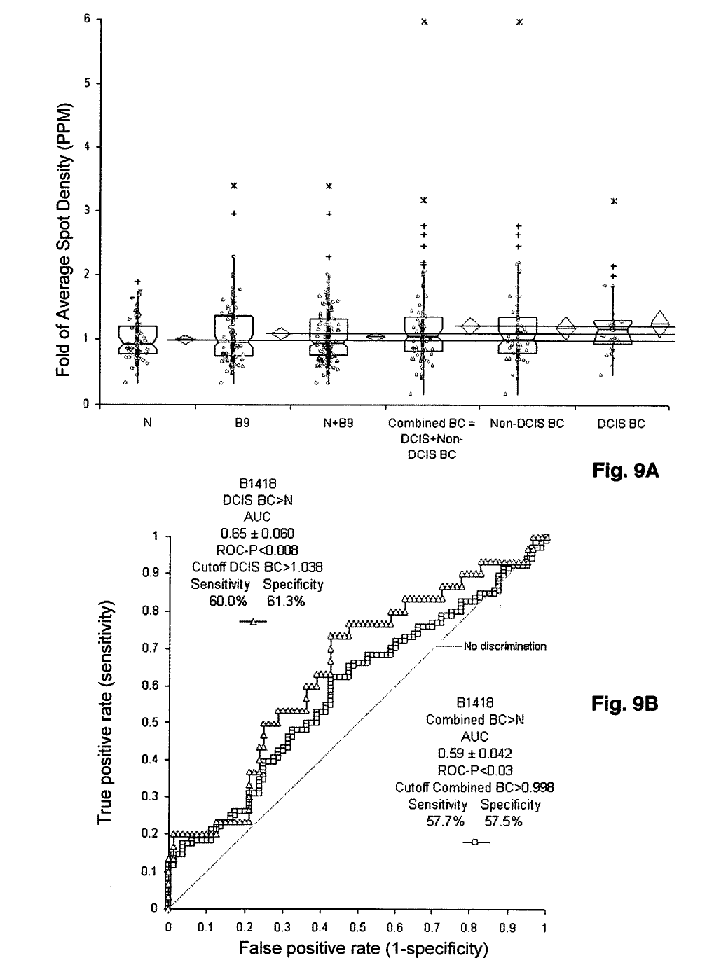

Table XXXVII: Summary statistics of the differential expression of Alpha-1-microglobulin protein spot B1418, equal to the fold of average normal blood serum concentration (concentration measured as protein spot density, PPM) as depicted in the graph in FIG. 9A.

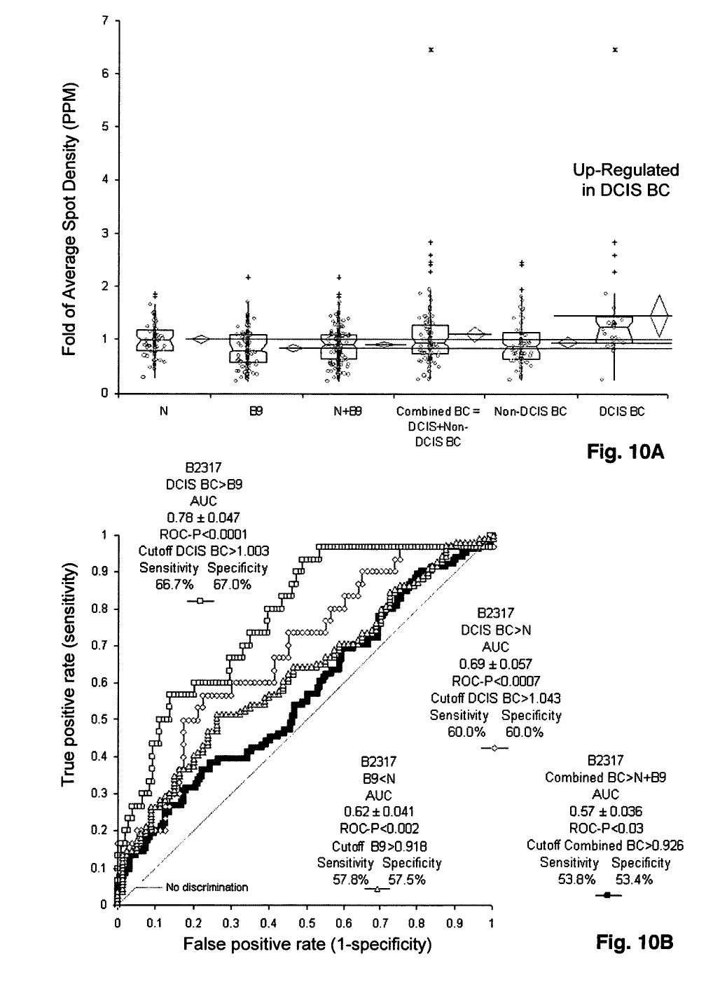

Table XXXVIII: Summary statistics of the differential expression of Apolipoprotein A1 protein spot B2317, equal to the fold of average normal blood serum concentration (concentration measured as protein spot density, PPM) as depicted in the graph in FIG. 10A.

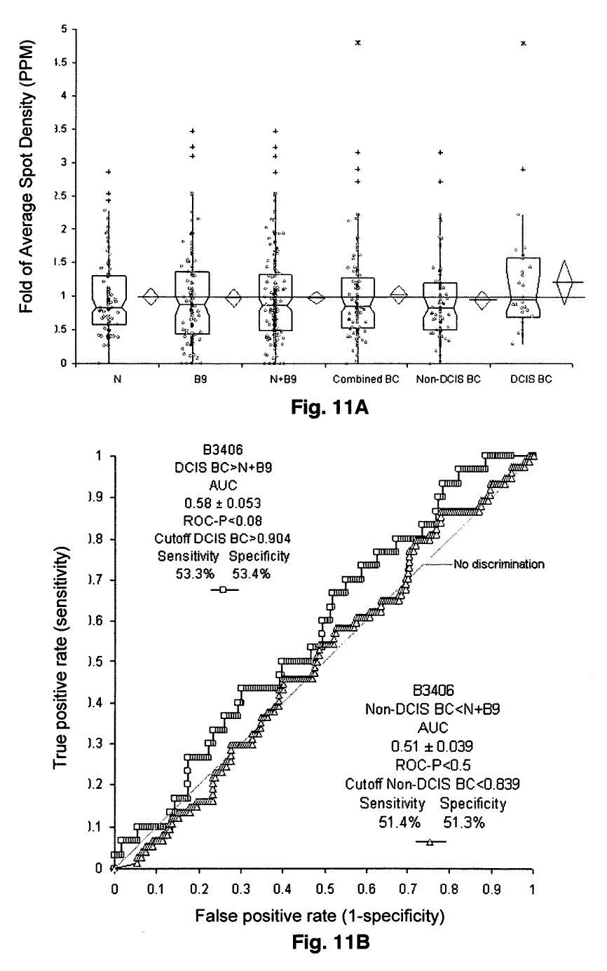

Table XXXIX: Summary statistics of the differential expression of Apolipoprotein E3 protein spot B3406, equal to the fold of average normal blood serum concentration (concentration measured as protein spot density, PPM) as depicted in the graph in FIG. 11A.

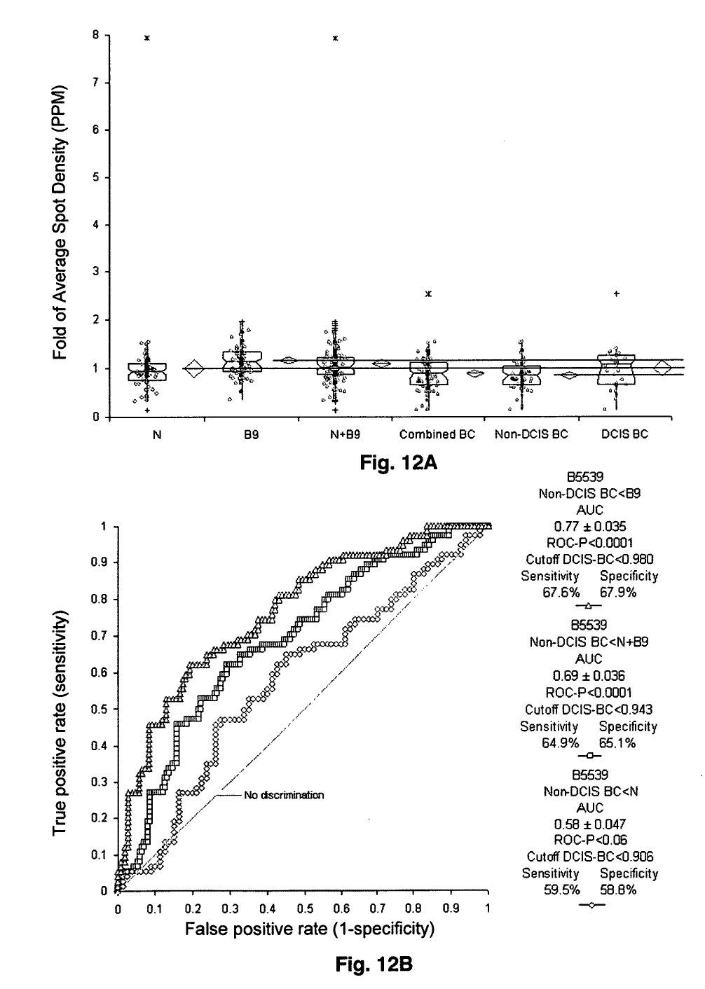

Table XL: Summary statistics of the differential expression of Serum albumin protein spot B5539, equal to the fold of average normal blood serum concentration (concentration measured as protein spot density, PPM) as depicted in the graph in FIG. 12A.

Table XLI: Summary statistics of the differential expression of protein Transferrin protein spot B6605, equal to the fold of average normal blood serum concentration (concentration measured as protein spot density, PPM) as depicted in the graph in FIG. 14A.

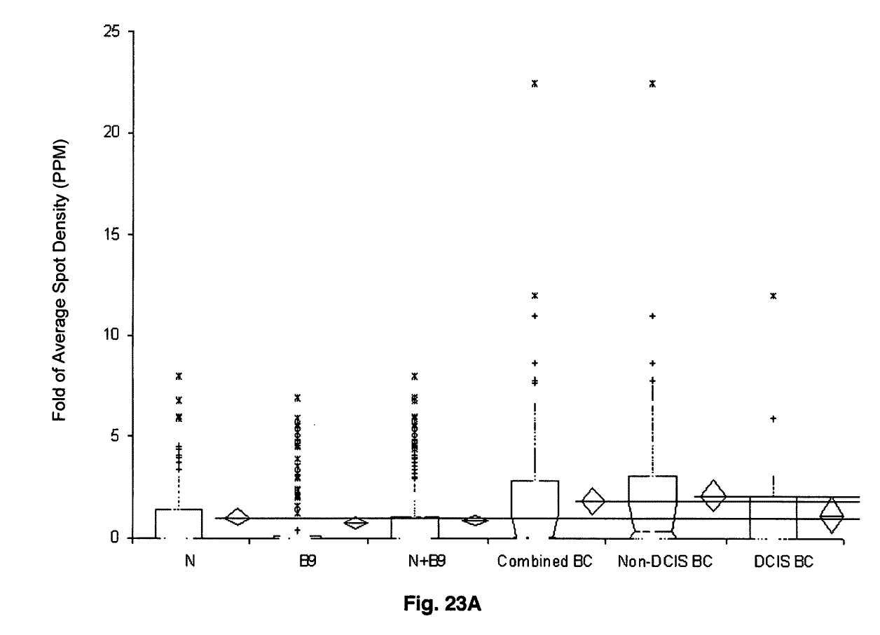

Table XLII: Summary statistics of the differential expression of Serotransferrin protein spot B5713, equal to the fold of average normal blood serum concentration (concentration measured as protein spot density, PPM) as depicted in the graph in FIG. 22A.

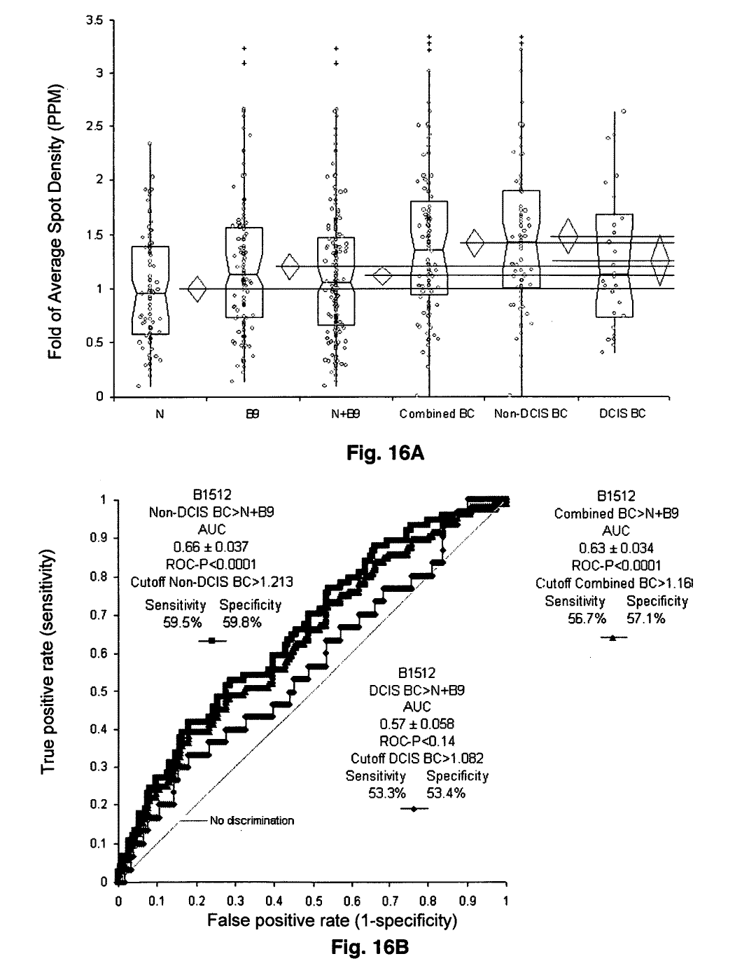

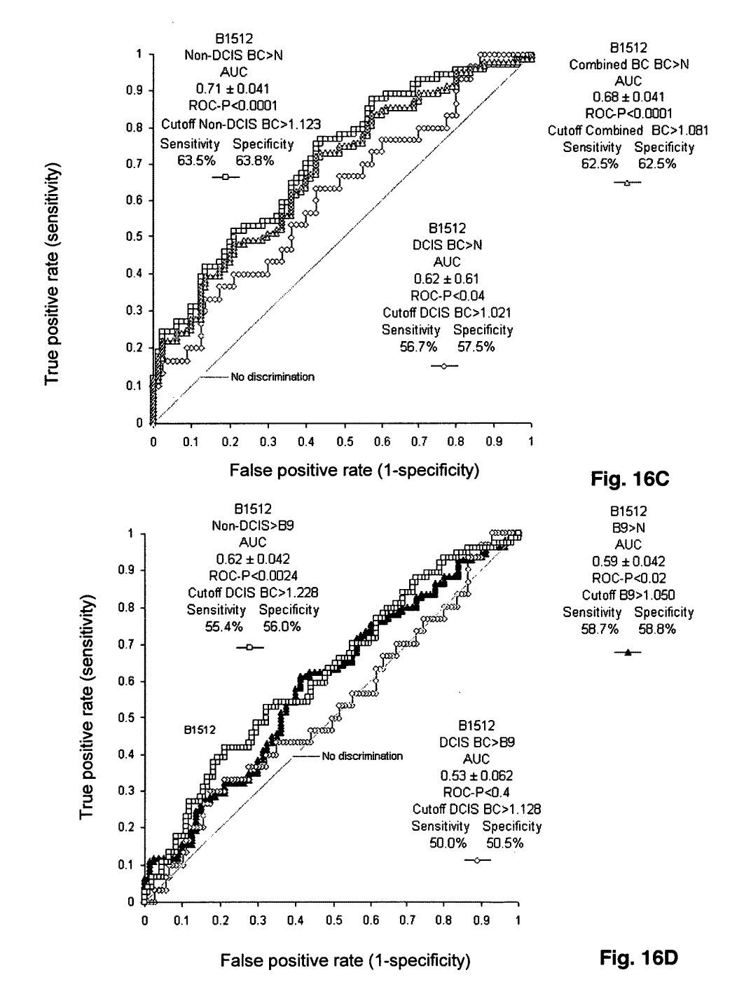

Table XLIII: Summary statistics of the differential expression of Haptoglobin protein spot B1512, equal to the fold of average normal blood serum concentration (concentration measured as protein spot density, PPM) as depicted in the graph in FIG. 16A.

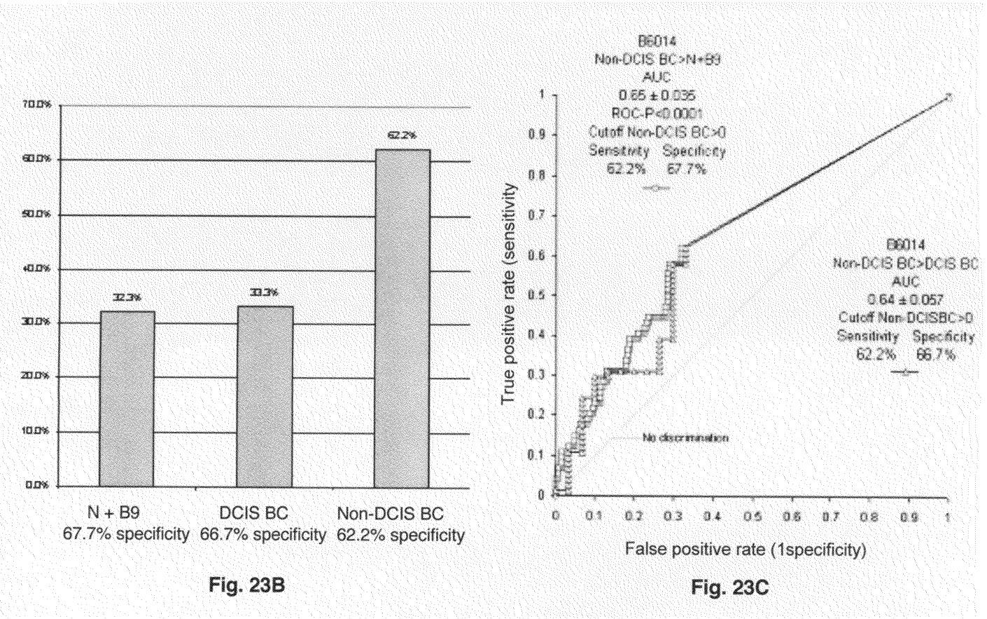

Table XLIV: Summary statistics of the differential expression of Haptoglobin protein spot B6014, equal to the fold of average normal blood serum concentration (concentration measured as protein spot density, PPM) as depicted in the graph in FIG. 23A.

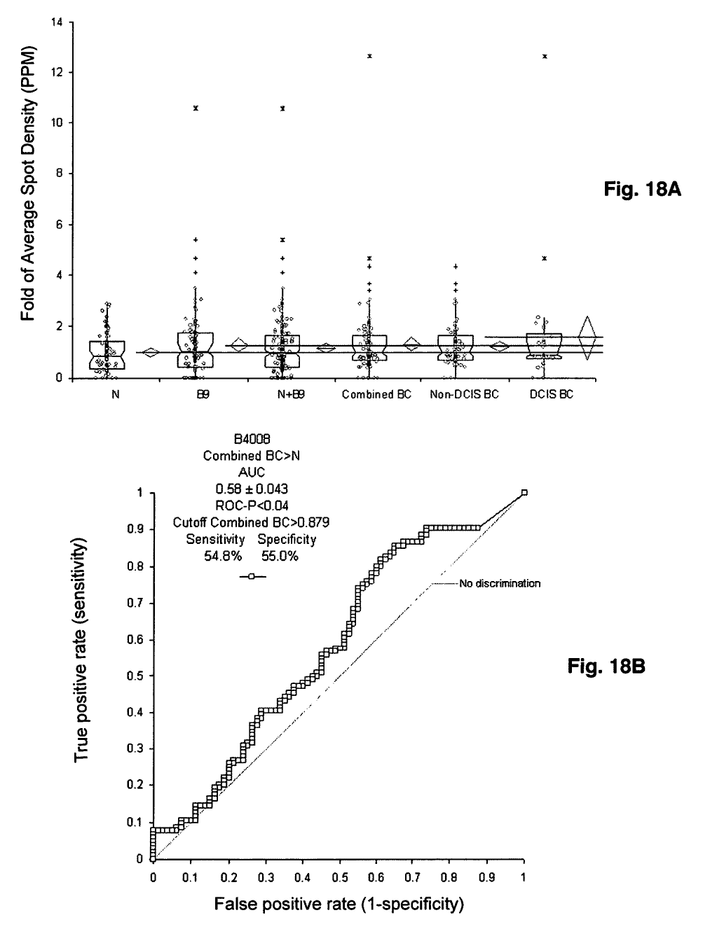

Table XLV: Summary statistics of the differential expression of Haptoglobin protein spot B4008, equal to the fold of average normal blood serum concentration (concentration measured as protein spot density, PPM) as depicted in the graph in FIG. 18A.

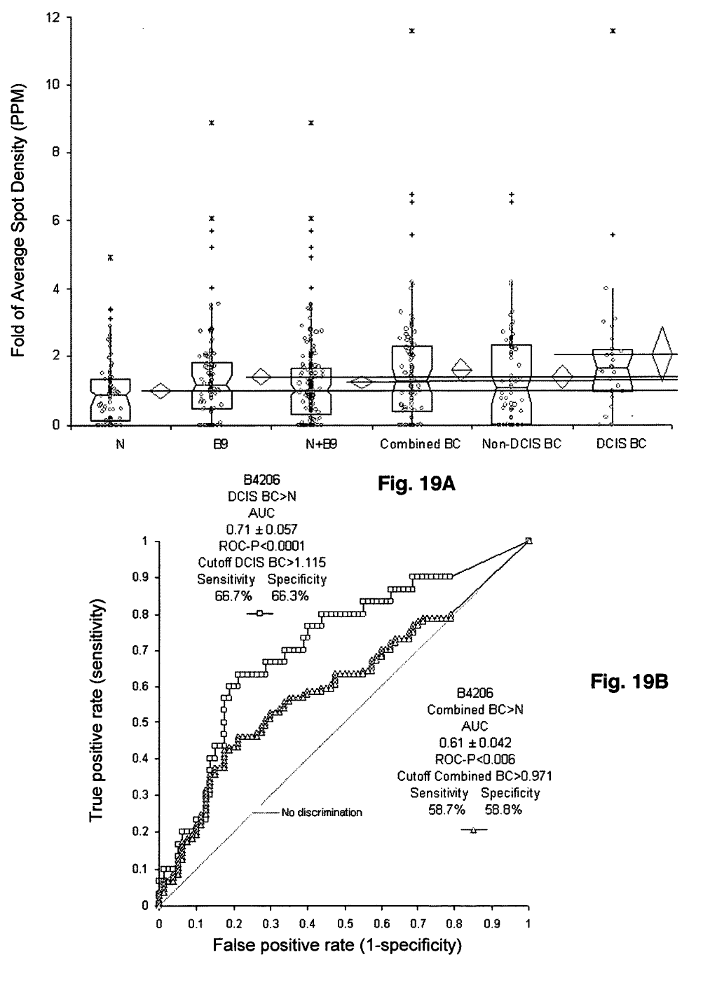

Table XLVI: Summary statistics of the differential expression of Haptoglobin protein spot B4206, equal to the fold of average normal blood serum concentration (concentration measured as protein spot density, PPM) as depicted in the graph in FIG. 19A.

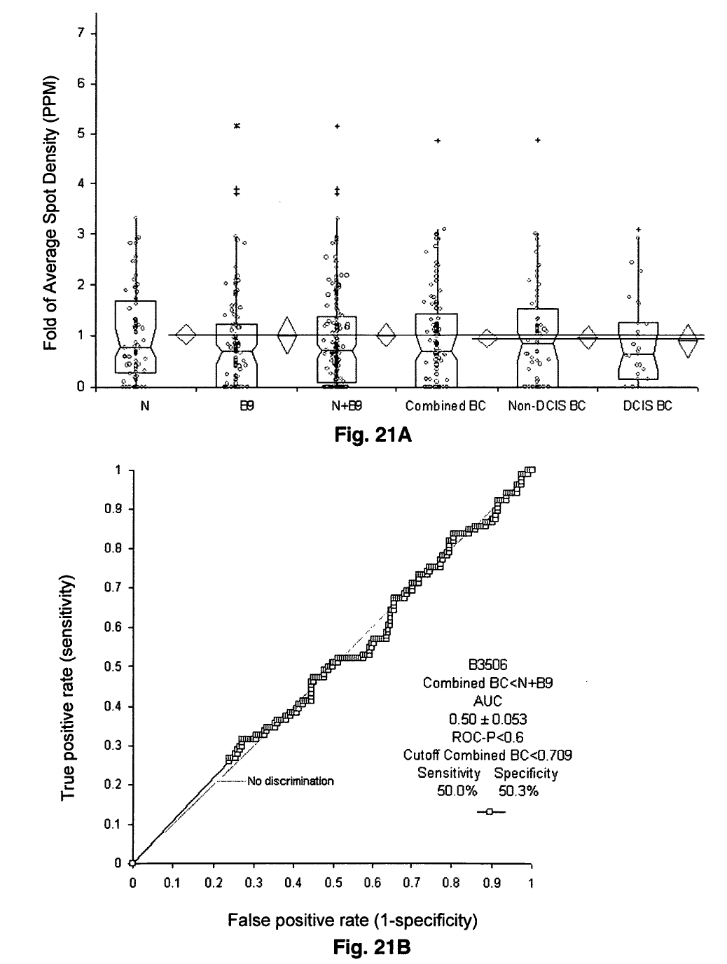

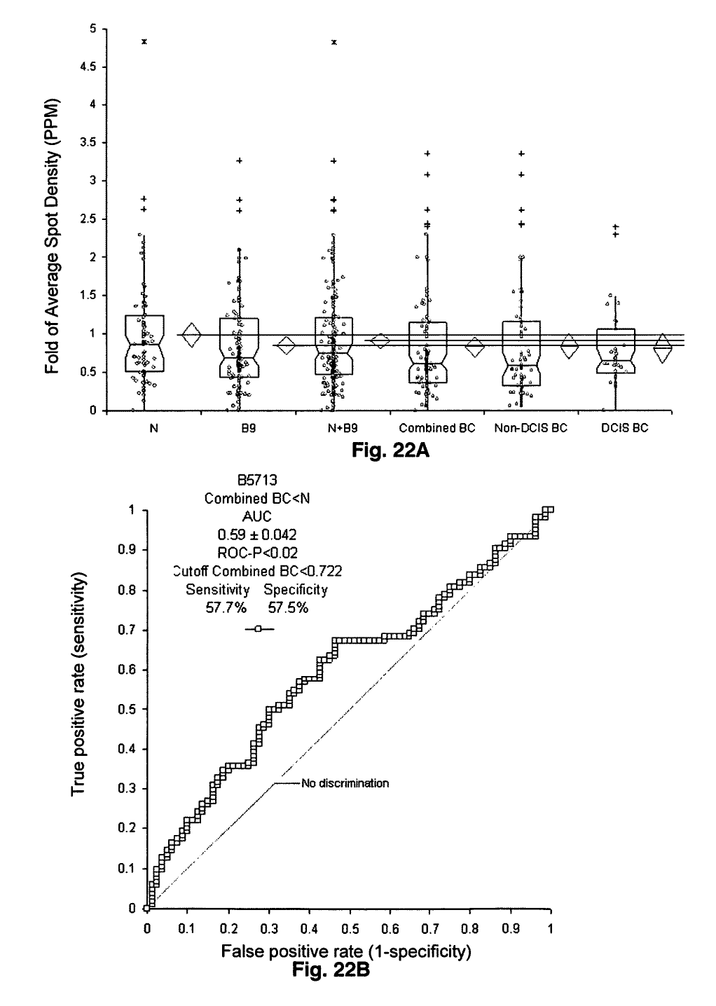

Table XLVII: Summary statistics of the differential expression of Haptoglobin related protein spot B3506, equal to the fold of average normal blood serum concentration (concentration measured as protein spot density, PPM) as depicted in the graph in FIG. 21A.

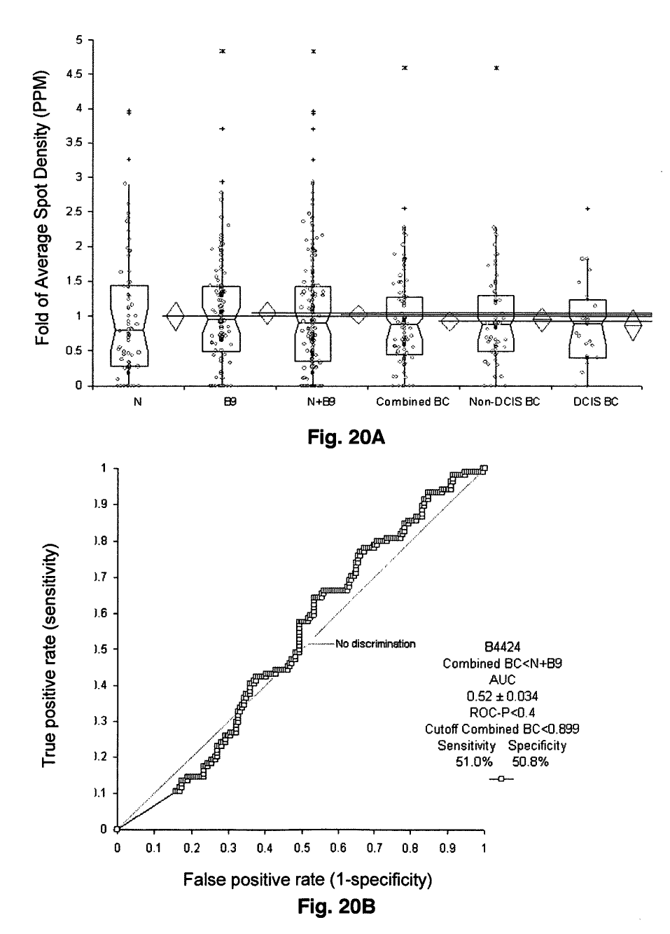

Table XLVIII: Summary statistics of the differential expression of Haptoglobin related protein spot B4424, equal to the fold of average normal blood serum concentration (concentration measured as protein spot density, PPM) as depicted in the graph in FIG. 20A.

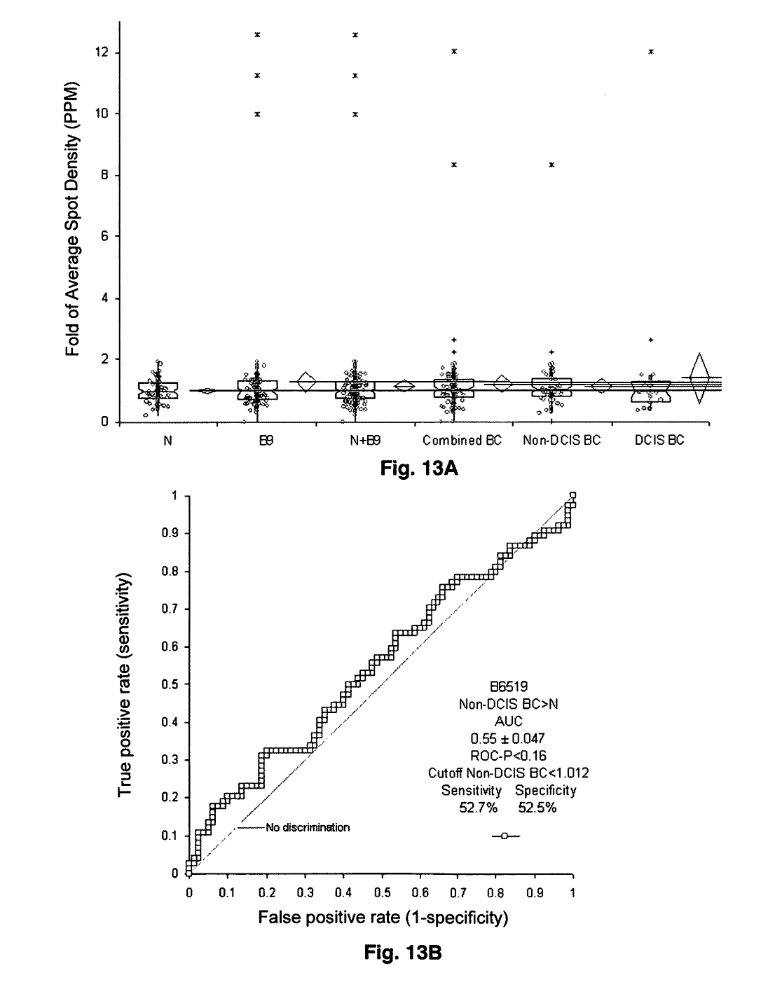

Table XLIX: Summary statistics of the differential expression of Lectin P35 3 protein spot B6519, equal to the fold of average normal blood serum concentration (concentration measured as protein spot density, PPM) as depicted in the graph in FIG. 13A.

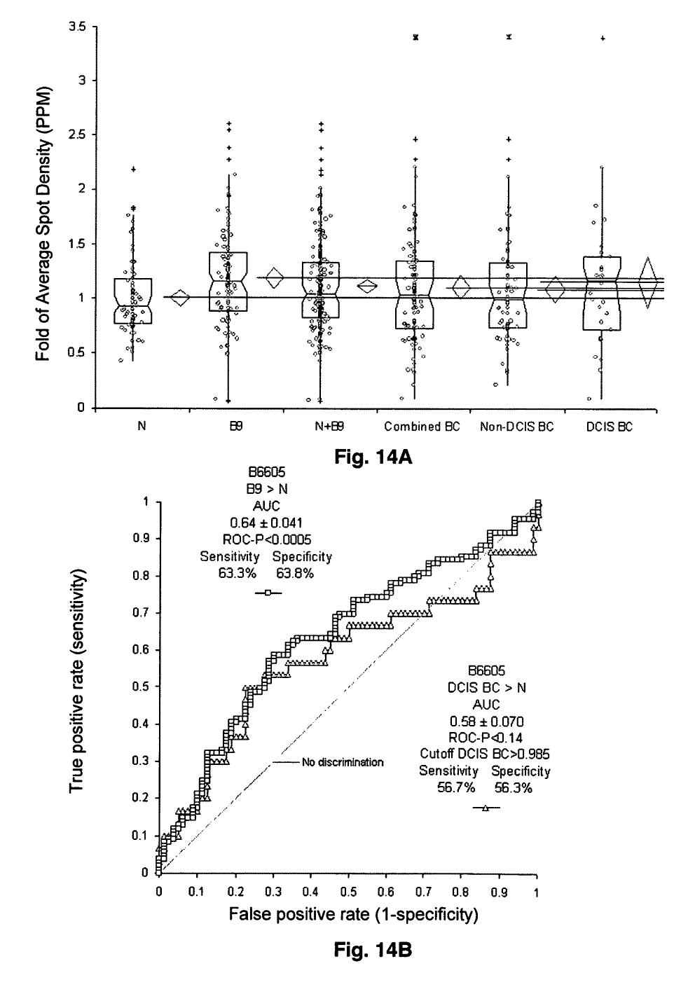

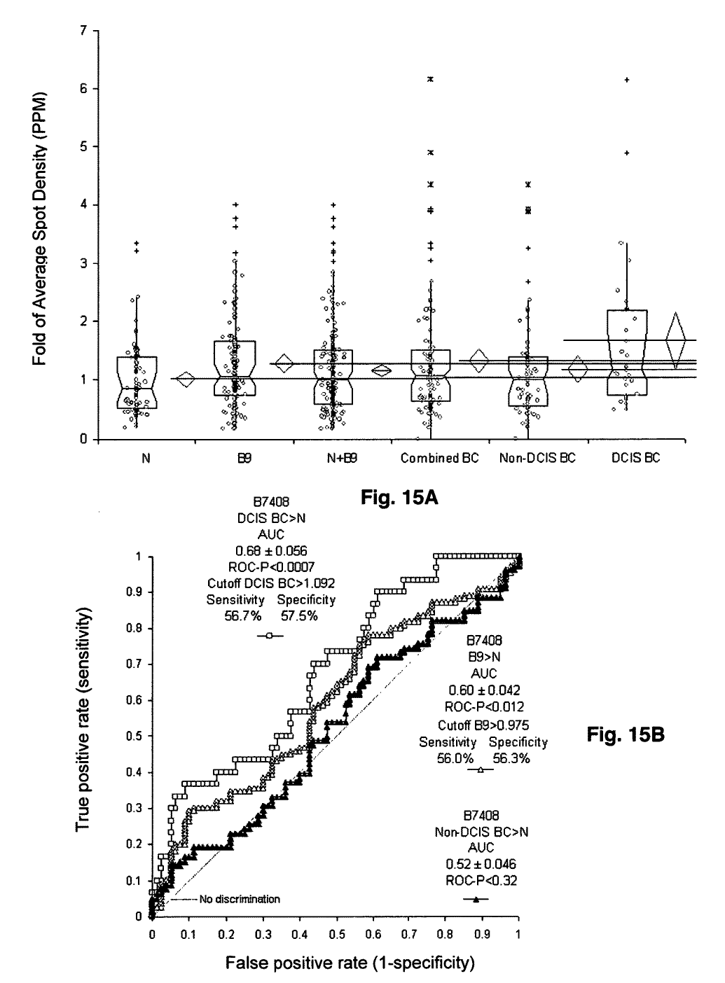

Table L: Summary statistics of the differential expression of Complement C4A gamma protein spot B7408, equal to the fold of average normal blood serum concentration (concentration measured as protein spot density, PPM) as depicted in the graph in FIG. 15A.

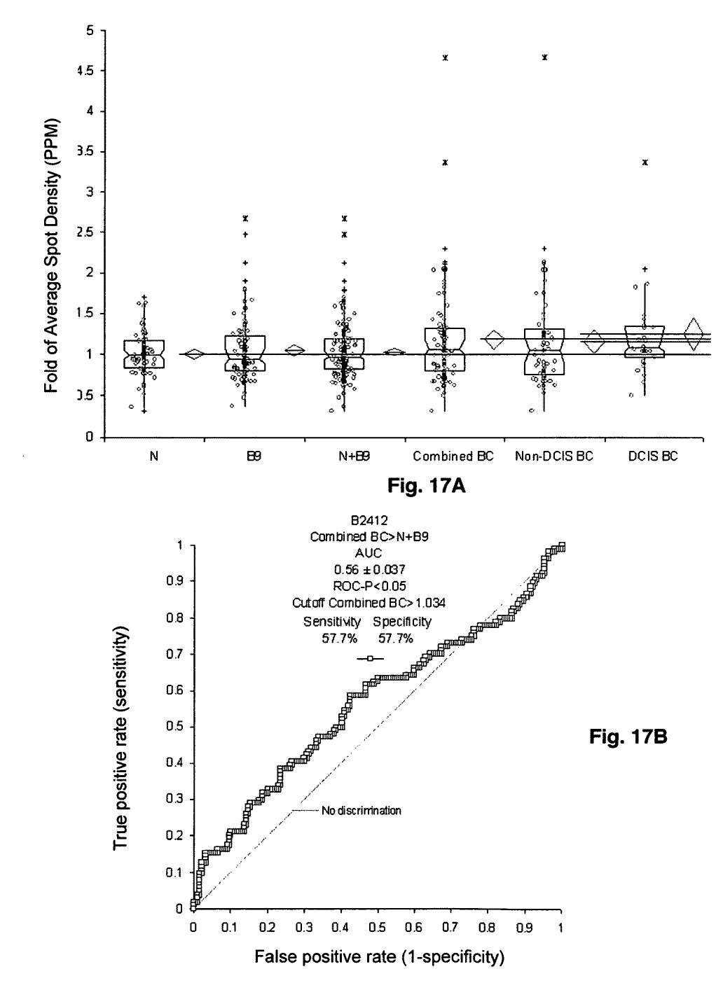

Table LI: Summary statistics of the differential expression of Apoptosis Inhibitor (CD5L) protein spot B2412, equal to the fold of average normal blood serum concentration (concentration measured as protein spot density, PPM) as depicted in the graph in FIG. 17A.

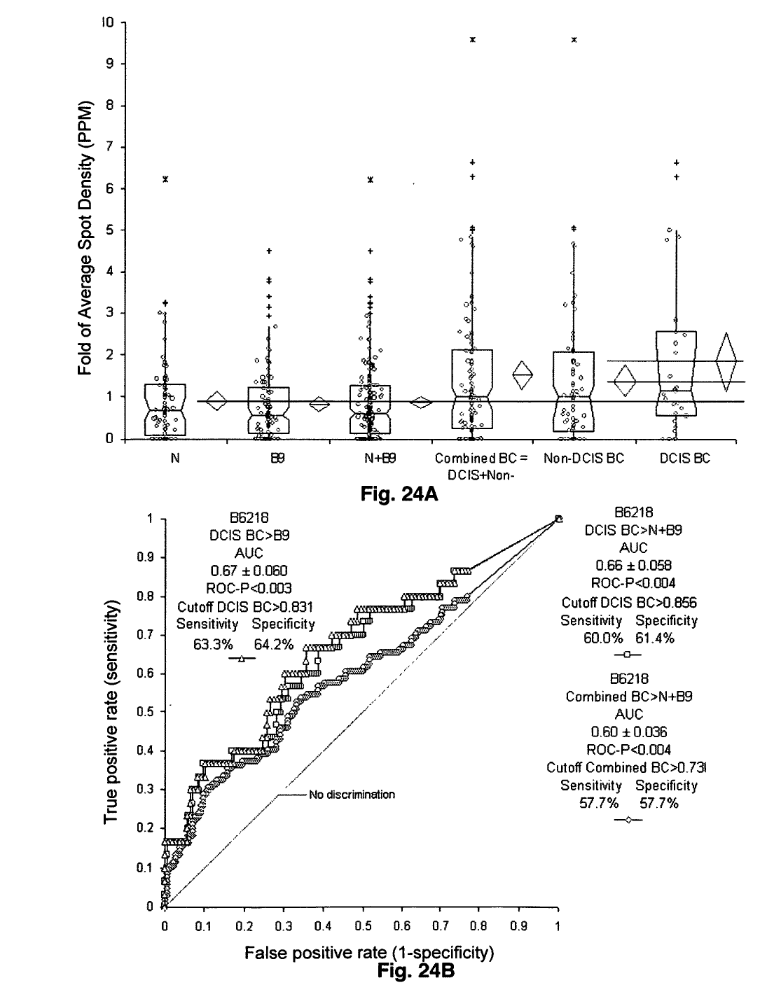

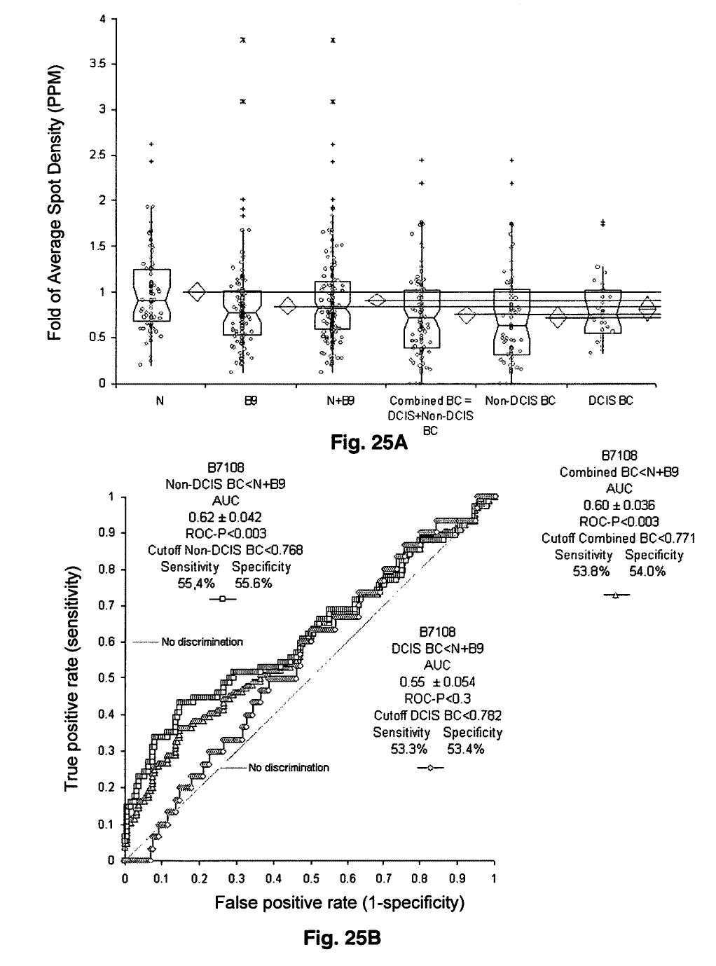

Table LII: Summary statistics of the differential expression of Nucleolar/ribosomal protein spot B6218, equal to the fold of average normal blood serum concentration (concentration measured as protein spot density, PPM) as depicted in the graph in FIG. 24A.

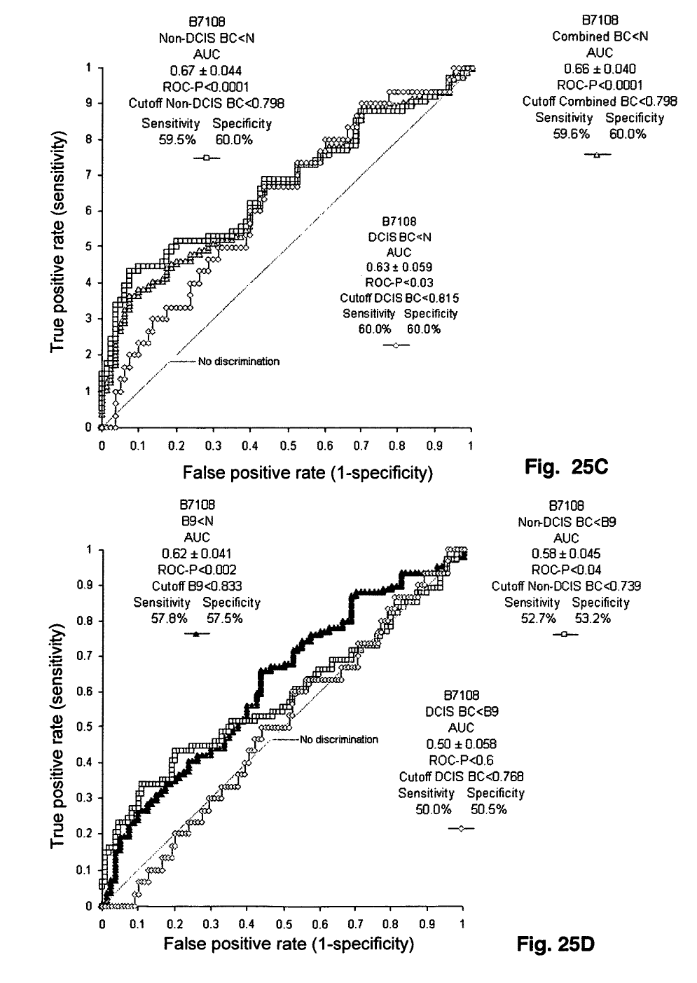

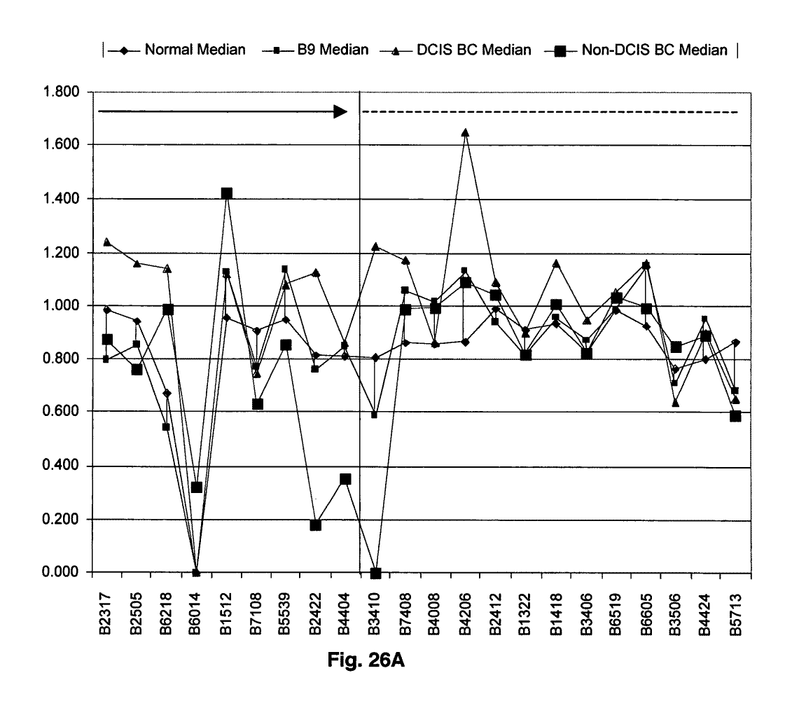

Table LIII: Summary statistics of the differential expression of Reticulon-4 protein spot B7108, equal to the fold of average normal blood serum concentration (concentration measured as protein spot density, PPM) as depicted in the graph in FIG. 25A.

Table LIV: Linear discriminant biostatistics of the differential expression in blood serum: a. the 9 Step Disk biomarkers and b. the total 22 breast cancer protein biomarkers.

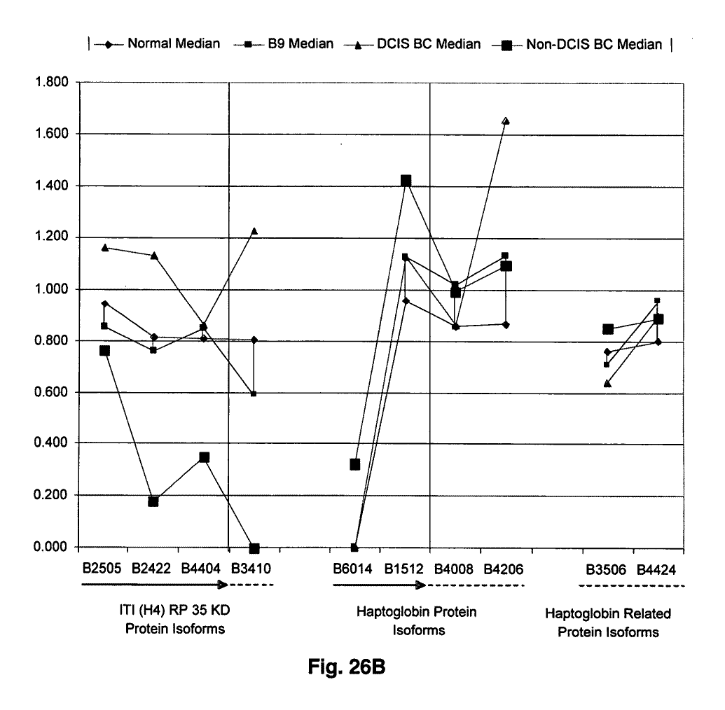

Table LV: The 22 breast cancer protein biomarker disease median profiles as depicted in the graphs in FIGS. 26A-26B.

### DESCRIPTION OF THE PREFERRED EMBODIMENTS

The present invention is a diagnostic assay for differentiating between patients having earlier and/or later stages of breast cancer, patients with benign breast disease and/or abnormalities, and normal control individuals. The method is based on the use of two-dimensional (2D) gel electrophoresis to separate the complex mixture of proteins found in blood serum and the quantitation of a group of identified biomarkers to differentiate between patients having earlier or later stages of breast cancer, patients with benign breast disease or abnormalities, and normal control individuals.

In the context of the present invention breast cancer consists of biopsy confirmed and histological staged disease. The breast cancer may be from a plurality of stages, wherein staging is the process physicians use to assess the size and location of a patient's cancer. Identifying the cancer stage is one of the most important factors in selecting treatment options. It should be noted that a patient may have more than stage of breast cancer at any one time, further complicating treatment and outcomes for the patient.

In the present invention, the stages of breast cancer are defined as shown in Table 1: In the context of the present invention, the “protein expression profile” corresponds to the steady state level of the various proteins in biological samples that can be expressed quantitatively. These steady state levels are the result of the combination of all the factors that control protein concentration in a biological sample. These factors include but are not limited to: the rates of transcription of the genes encoding the mRNAs; processing of the mRNAs into mRNAs; The rates of splicing and the splicing variations during the processing of the mRNAs into mRNAs which govern the relative amounts of the protein sequence isoforms; the rates of processing of the various mRNAs by 3′-polyadenylation and 5′-capping; the rates of transport of the mRNAs to the sites of protein synthesis; the rate of translation of the mRNA's into the corresponding proteins; the rates of protein post-translational modifications, including but not limited to phosphorylation, nitrosylation, methylation, acetylation, glycosylation, poly-ADP-ribosylation, ubiquitinylation, and conjugation with ubiquitin Like proteins; the rates of protein turnover via the ubiquitin-proteosome system and via proteolytic processing of the parent protein into various active and inactive subcomponents; the rates of intracellular transport of the proteins among compartments, such as but not limited to the nucleus, the lysosomes, golgi, the membrane, and the mitochondrion; the rates of secretion of the proteins into the interstitial space; the rates of secretion related protein processing; and the stability and rates of proteolytic processing and degradation of the proteins in the biological sample before and after the sample is taken from the patient.

In the context of the present invention, a “biomarker” corresponds to a protein or protein fragment present in a biological sample from a patient, wherein the quantity of the biomarker in the biological sample provides information about whether the patient exhibits an altered biological state such as earlier breast cancer such as ductal carcinoma in situ (DCIS, Stage 0), later breast cancer (Invasive, Stages I, II, III, IV), or combinations thereof, such as breast cancer that includes ductal carcinoma in situ (DCIS DCIS-BC), or breast cancer that does not include ductal carcinoma in-situ (Non-DCIS-BC), or benign breast disease or abnormalities (B9).

A “normal” sample is a sample, preferably a normal serum sample, is taken from an individual with no known breast disease and/or no known breast abnormalities.

The present invention is based on the quantification of specified proteins. Preferably the proteins are separated and identified by 2D gel electrophoresis. In the past, this method has been considered highly specialized, labor intensive and non-reproducible.

Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, nitrosylation, ubiquitination, conjugation with ubiquitin-Like proteins, acetylation, and glycosylation. These are important variables in cell regulatory processes involved in disease states.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

### Materials and Methods:

### Sample Collection and Preparation

Serum samples were prepared from blood acquired by venipuncture. The blood was allowed to clot at room temperature for 30-60 minutes, centrifuged at 1200×g for 15 minutes, and the separated serum was divided into aliquots, and frozen at −40° C. or below until shipment. Samples were shipped on dry ice and were delivered within 24 hours of shipping.

Once the serum samples were received, logged in, and assigned a sample number; they were further processed in preparation for 2D gel electrophoresis. All samples were stored at −80° C. or below. When the serum samples were removed from storage, they were placed on ice for thawing and kept on ice for further processing.

### Separation of Proteins in Patient Samples

The serum protein from patients and normal control subjects analyzed in the present invention were separated using 2D gel electrophoresis. Other various techniques known in the art for separating proteins can also be used. These other techniques include but are not limited to gel filtration chromatography, ion exchange chromatography, reverse phase chromatography, affinity chromatography, or any of the various centrifugation techniques well known in the art. In some cases, a combination of one or more chromatography or centrifugation steps may be combined via electrospray or nanospray with mass spectroscopy or tandem mass spectroscopy, or any protein separation technique that determines the pattern of proteins in a mixture either as a one-dimensional, two-dimensional, three-dimensional or multi-dimensional pattern or list of proteins present.

### Two Dimensional Gel Electrophoresis of Samples

Preferably the protein profiles of the present invention are obtained by subjecting biological samples to two-dimensional (2D) gel electrophoresis to separate the proteins in the biological sample into a two-dimensional array of protein spots.

Two-dimensional gel electrophoresis is a useful technique for separating complex mixtures of proteins and can be performed using a variety of methods known in the art (see, e.g., U.S. Pat. Nos. 5,534,121; 6,398,933; and 6,855,554).

Preferably, the first dimensional gel is an isoelectric focusing gel and the second dimension gel is a denaturing polyacrylamide gradient gel.

Proteins are amphoteric, containing both positive and negative charges and like all ampholytes exhibit the property that their charge depends on pH. At low pH (acidic conditions), proteins are positively charged while at high pH (basic conditions) they are negatively charged. For every protein there is a pH at which the protein is uncharged, the protein's isoelectric point. When a charged molecule is placed in an electric field it will migrate towards the opposite charge.

In a pH gradient such as those used in the present invention, containing a reducing agent such as dithiothreitol (DTT), a protein will migrate to the point at which it reaches its isoelectric point and becomes uncharged. The uncharged protein will not migrate further and stops. Each protein will stop at its isoelectric point and the proteins can thus be separated according to their isoelectric points. In order to achieve optimal separation of proteins, various pH gradients may be used. For example, a very broad range of pH, from about 3 to 11 or 3 to 10 can be used, or a more narrow range, such as from pH 4 to 7 or 5 to 8 or 7 to 10 or 6 to 11 can be used. The choice of pH range is determined empirically and such determinations are within the skill of the ordinary practitioner and can be accomplished without undue experimentation.

In the second dimension, proteins are separated according to molecular weight by measuring mobility through a uniform or gradient polyacrylamide gel in the detergent sodium dodecyl sulfate (SDS). In the presence of SDS and a reducing agent such as dithiothreitol (DTT), the proteins act as though they are of uniform shape with the same charge to mass ratio. When the proteins are placed in an electric field, they migrate into and through the gel from one edge to the other. As the proteins migrate though the gel, individual proteins move at different speeds with the smaller ones moving faster than the larger ones. This process is stopped when the fastest moving components reach the other side of the gel. At this point, the proteins are distributed across the gel with the higher molecular weight proteins near the origin and the low molecular weight proteins near the other side of the gel.

It is well known in the art that various concentration gradients of acrylamide may be used for such protein separations. For example, a gradient of about 5% to 20% may be used in certain embodiments or any other gradient that achieves a satisfactory separation of proteins in the sample may be used. Other gradients would include but not be limited to about 5 to 18%, about 6 to 20%, about 8 to 20%, about 8 to 18%, about 8 to 16%, about 10 to 20%, or any range as determined by one of skill.

The end result of the 2D gel procedure is the separation of a complex mixture of proteins into a two dimensional array, a pattern of protein spots, based on the differences in their individual characteristics of isoelectric point and molecular weight.

### Reagents

Protease inhibitor cocktail were from Roche Diagnostics Corporation (Indianapolis, Ind.), Protein assay and purification reagents were from Bio-Rad Laboratories (Hercules, Calif.). Immobilon-P membranes and ECL reagents were from Pierce (Rockford, Ill.). All other chemicals were from Sigma Chemical (St. Louis, Mo.).

### 2D Gel Standards

Purified proteins having known characteristics are used as internal and external standards and as a calibrator for 2D gel electrophoresis. The standards consist of seven reduced, denatured proteins that can be run either as spiked internal standards or as external standards to test the ampholyte mixture and the reproducibility of the gels. A set mixture of proteins (the “standard mixture”) is used to determine pH gradients and molecular weights for the two dimensions of the electrophoresis operation. As shown below, Table II lists the isoelectric point (pI) values and molecular weights for the proteins included in a standard mixture.

In addition, standard mixtures such as Precision Plus Protein Standards (Bio-Rad Laboratories), a mixture of 10 recombinant proteins ranging from 10-250 kD, are typically added as external molecular weight standards for the second dimension, or the SDS-PAGE portion of the system. The Precision Plus Protein Standards have an r² value of the R_{f} vs. log molecular weight plot of >0.99.

### Separation of Proteins in Serum Samples

An appropriate amount of isoelectric focusing (IEF) loading buffer (LB-2), was added to the diluted serum sample, incubated at room temperature and vortexed periodically until the pellet was dissolved to visual clarity. The samples were centrifuged briefly before a protein assay was performed on the sample.

Approximately 100 μg of the serum proteins were suspended in a total volume of 184 μl of IEF loading buffer containing 5 M urea, 2 M Thiourea, 1% CHAPS, 2% ASB-14, 0.25% Tween 20, 100 mM DTT, 1% ampholytes pH 3-10, 5% glycerol, 1×EDTA-free protease inhibitor cocktail and 1 μl Bromophenol Blue as a color marker to monitor the process of gel electrophoresis. Each sample was loaded onto an 11 cm IEF strip (Bio-Rad Laboratories), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad Laboratories running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

### Staining and Analysis of the 2D Gels

Once the 2D gel patterns of the serum samples are obtained, the protein spots resolved in the gels are visualized with either a fluorescent or colored stain. In the preferred embodiment, the fluorescent dye Lava Purple (Fluorotechnics) is the fluorescent stain. In another embodiment, another fluorescent stain, such as SyproRuby™ (Bio-Rad Laboratories) is employed. Once the protein spots are stained, the gel is scanned by a digital fluorescent scanner. In a preferred embodiment the FLA-7000 (Fujifilm) is the fluorescent scanner. In another embodiment, another fluorescent scanner, such as an FX-Imager (Bio-Rad Laboratories) is employed, or when visible dyes, such as silver or Coomassie Blue, are employed, a digital visible light scanner, such as a GS-800 densitometer (Bio-Rad Laboratories) is employed. The fluorescent or visible digital image of the protein spot pattern of the 2D gel, i.e. a protein expression profile of the sample, is thus obtained.

The digital image of the scanned gel is processed using PDQuest™ (Bio-Rad Laboratories) image analysis software to first detect the proteins, locate the selected biomarkers, and then to quantitate the protein in each of the selected spots. The scanned image is cropped and filtered to eliminate artifacts, using the image editing control. Individual cropped and filtered images are then placed in a matched set for comparison to other images and controls.

This process allowed quantitative and qualitative spot comparisons across gels and the determination of protein biomarker molecular weight and isoelectric point values. Multiple gel images were normalized to allow an accurate and reproducible comparison of spot quantities across two or more gels. The gels were normalized using the “total of all valid (detected and confirmed by the operator) spots method” in that a small percentage of the 1200 protein spots detected and verified change between serum samples, and that all spots detected and verified is a good estimate to correct for any differences in total protein amount applied to each gel. The quantitative amounts of the selected biomarkers present in each sample were then exported for further analysis using statistical programs.

### Tryptic Digestion, MALDI/MS, and LC-MS/MS

Following software analysis, unique spots were excised from the gel using the ProteomeWorks™ robotic spot cutter (Bio-Rad). In-gel spots were subjected to proteolytic digestion on a ProGest™ (Genomic Solutions, Ann Arbor, Mich.). A portion of the resulting digest supernatant was used for MALDI/MS analysis. Peptide solutions were concentrated and desalted using μ-C18 ZipTips™ (Millipore). Peptides were eluted with MALDI matrix alpha-cyano 4-hydroxycinnamic acid prepared in 60% acetonitrile, 0.2% TFA. Samples were robotically spotted onto MALDI chip, using ProMS™ (Genomic Solutions, Ann Arbor, Mich.).

MALDI/MS data was acquired on an Applied Biosystems Voyager DE-STR instrument and the observed m/z values were submitted to ProFound (Proteometrics software package) for peptide mass fingerprint searching using NCBInr database.

For LC/MS/MS, samples were analyzed by nano-LC/MS/MS on a Micromass Q-TOF 2. Aliquots of 15 μl of hydrolysate were processed on a 75 mm C18 column at a flow rate of 200 nL/min. MS/MS data were searched using a local copy of MASCOT, using peptide mass tolerance of ±100 ppm and fragment mass tolerance of ±0.1 Da, fixed modification of carbamidomethyl (C) and variables, including oxidation (M), acetyl (N-term), Pyro-glu (N-term Q), Pyro-glu (N-term E) and max missed cleavages of trypsin of 1.

### Biostatistical Analysis

Statistical significance of differences in biomarker blood serum concentrations between different patient and control groups is performed using methods well known in the art, such as Box and Whiskers plots, Receiver Operator Characteristics (ROC), and analysis of variance, employing a standard off the shelf software package, such as “Analyze-it” in Microsoft XL.

Discriminant analysis is a well-validated multivariate analysis procedure. Discriminant analysis identifies sets of linearly independent functions that will successfully classify individuals into a well-defined collection of groups. The statistical model assumes a multivariate normal distribution for the set of biomarkers identified from each disease group. Let _ be the p-tuple vector of biomarkers from the i^{th} patient in the j^{th} group, j=1, 2 Let ^{—} be the p-tuple centroid of the j^{th} group, made up of the mean biomarker values from the j^{th} disease group. S is the estimate of the within group variance-covariance matrix. The discriminant function is then that set of linear functions determined by the vector a that maximizes the quantity:

$$\frac{{n}_{1} + {n}_{2}}{{n}_{1} {n}_{2}} \frac{{[ {a}^{′} ( {x}_{1} - {x}_{2} ) ]}^{2}}{{\underline{a}}^{′} Sa}$$

The outcome of the discriminant analysis is a collection of m−1 linear functions of the biomarkers (m) that maximize the ability to separate individuals into disease groups. The vector a is the p-tuple vector which contains the coefficients that, when multiplied by an individual's biomarkers, produces the linear discriminant function, or index that is used to classify that individual.

In general, if there are m biomarkers, there will be a maximum of (m−1, g−1) discriminant functions where g is the number of groups. Let aⱼ (k) be the k^{th} p-tuple discriminant function. Then the value of that discriminator for the i^{th} patient is aⱼ (k)′xᵢ. Thus for each patient there are k such values computed, which are used in a classification analysis. The discriminant functions themselves are linearly independent, i.e., for each pair of the m discriminant functions, aⱼ(k) and aⱼ(l), then, aⱼ(k)′aⱼ(l)=0. Thus, the m−1 discriminant functions provide incremental and non-redundant discriminant ability.

Identifying the discriminant function involves identifying the coefficients A from the linear algebraic system of equations |H−λᵢ(H+E)|=0 where H and E are the one way analysis of variance hypotheses and error matrices respectively. It is this computation that is provided by SAS. SAS identifies the collection of best discriminators using a forward entry procedure where the p-value to enter and the p value to stay in the model are each 0.15.

While the discrimination procedure is fairly robust in the presence of mild departures from the normality assumption, it is very sensitive to the assumption of homogeneity of variance. This means that the variance-covariance matrices of the groups between which discrimination is sought must be equal. In this circumstance, these variance-covariance matrices can be pooled. However, in the situation where the variance-covariance matrices are not equal (multivariate heteroscedasticity), this pooling procedure is sub-optimal. In this circumstance, the individual variance-covariance matrices are used.

The use of the two within-group variance-covariance matrices is an important complication in the computation of discriminant functions. When the homoscedasticity assumption is appropriate, the within group variance-covariance matrices can be pooled, producing a linear discriminant function. The use of the within-group variance-covariance matrices produces a quadratic discriminant function, (i.e., where the discriminant function is a function of the squares of the proteomic measures). Both linear and quadratic statistical functions are illustrated in the embodiments of this invention.

### Classification Analysis

Discriminant analysis was applied to the training set, from which the contribution of each individual biomarker was determined. The SAS® statistical software program was then used to determine the linear combinations of biomarkers that provided an optimum classification of individuals into disease groups. Alternatively, the programmer manually selected different combinations of biomarkers to be incorporated into a linear or quadratic discriminant function to optimize the classification of individuals into disease groups.

The output of discriminant analysis (DA) is a classification table that permits the calculation of clinical sensitivity, specificity, positive predictive value (PPV) and negative predictive value (NPV):
Clinical Sensitivity is how often the test is positive in diseased patients.
Clinical Specificity is how often the test is negative in non-diseased individuals.
Negative Predictive Value (NPV) is the probability that the patient will not have the disease when restricted to all individuals who test negative.
Positive Predictive Value (PPV) is the probability that the patient has the disease when restricted to those individuals who test positive.

NPV and PPV were not assessed in the case of the present study as these values are dependent upon patient mix and the present study used different numbers of patients in each category, due to sample availability.

### 2D Gel Electrophoretic Controls

Representative samples from individuals with known cases of breast cancer, benign breast disease, or normal controls, were run as positive and negative reference controls. Serum containing all of the selected biomarkers was also provided as a reference standard. A reference control was periodically run as an external standard and for tracking overall performance and reproducibility. In addition, 2D gel images from samples classified as breast cancer, benign breast disease, or normal controls, were used for reference. The spot locations for the selected biomarkers were illustrated in FIG. 1.

### Samples Analyzed

The present invention is a two-dimensional gel electrophoresis assay of patient blood serum samples, employing the 22 biomarker spots, combined with multivariate biostatistics, is used to distinguish between subjects with normal breasts, patients with benign breast disease, and patients with breast cancer.

The 2D gel electrophoresis of the human blood serum samples of this study separated >1200 spots in the pH 5-8 range, 22 of which (FIGS. 1 and 8A-8B, numbered spots: B1322, B1418, B2317, B2422, B2525, B3406, B3410, B4404, B5539, B6505, B6519, B7408, B1512, 2412, B4008, B4206, B3506, B4424, B5713, B6014, B6218, and B7108) displayed differences in serum concentrations between samples from normal subjects, patients with benign breast disease or abnormalities, and patients with breast cancer.

When the 22 biomarker spots were robotically excised, subjected to in-gel trypsin digestion and the peptides analyzed by LC-MS/MS fingerprint identification, (Tables III), comparison of the 2D gel measured and the protein sequence calculated masses and isoelectric points of the biomarker spots, with the peptides identified by LC-MS/MS, indicated that some of the biomarker protein spots appear on 2D gels as smaller components of parent molecules, i.e. smaller than the original translation products of the mRNA, whereas others are the full length translated products, including those with additional molecular weight contribution from post-synthetic modifications, such as glycosylation, etc (FIGS. 1, 6, 7A-7B, Tables III-VI, VII-XXXII, SEQ ID NOS: 1-22).

Spot identification by LC MS/MS of in-gel trypsin digests, and pI and Molecular Weight estimations from 2D gels and amino acid sequences (FIGS. 1, 6, 7A-7B, Tables III-VI) indicated that biomarker protein spots B2422, B2505, B3410, and B4404 (FIGS. 1, 6) correspond to electrophoretic variants of the 35 KD processing product of Inter-alpha-trypsin inhibitor heavy chain (H4) related protein, isoforms 1 and 2 (Tables VI, X-XII, SEQ ID NOS: 4-5).

Normal Controls Vs. Benign Breast Abnormalities Vs. Breast Cancer

These four spots corresponding to the 35 KD isoforms of the Inter-alpha-trypsin inhibitor Heavy Chain (H4) related protein, individually FIG. 2[[ ]]A-2D) and collectively (=B25422+B2505+B3410+B4404, FIG. 3A), demonstrated differences in blood serum concentrations between normal controls (N), patients with benign breast disease or abnormalities (B9), and patients with breast cancer (BC) (Table XXXIII and XXXIV).

FIG. 2 illustrates that when these four spots corresponding to the 35 KD isoforms of the Inter-alpha-trypsin inhibitor Heavy Chain (H4) related protein were analyzed for individual performance by 2D gel electrophoresis (FIGS. 2[[: ]]A: B2422; 2B: B2505; 2C: B3410; 2D: B4404), three of the four, A: B2422; B: B3410; and D: B4404, demonstrated down-shifts in blood serum concentration in breast cancer patients (BC) vs. normal controls (N) and patients with benign breast disease or abnormalities (B9) (Table XXXIII a, c, d). Conversely, the other isoform spot (B2505, FIG. 2B) actually displayed an increase in concentration in breast cancer patients (Table XXXIII b).

FIG. 3A and Table XXXIII e illustrates that when all four isoforms are analyzed as the total sum, the combined effect is a more modest down-shift (Table XXXIII e), masking the differences in performance between the isoforms seen in FIGS. 2A-2B. Furthermore, as also illustrated in FIG. 3A and Table XXXIV, there is a difference between the concentrations in the retrospective samples vs. the concentrations in the prospective samples, such that the normal (N) and breast cancer (BC) prospective samples both have higher concentrations of the combined 35 KD isoforms of the Inter-alpha-trypsin inhibitor Heavy Chain (H4) related protein biomarkers (sum of the concentrations of B2422+B2505+B3410+B4404), than that of the retrospective samples (Table XXXIV). This renders the retrospective samples no longer capable of performing as a model to diagnose the prospective samples (FIG. 3A arrow). This in part explains why so many protein biomarkers, originally discovered in retrospective biological samples, such as blood serum stored in freezers, fail to validate clinically upon fresh prospective samples.

While the use of absolute values of concentrations of the protein biomarkers (for example 2D gel spot density, PPM) do not provide for consistency between retrospective and prospective databases, another embodiment of the invention consists of determining the differential expression on the basis of the fold value of the normal concentrations, wherein:
Differential Expression: The deviation in biomarker concentration from the normal state as a function of disease, and wherein:

$$\begin{matrix}Differential Expression = Fold of average normal biomarker \\ protein concentration \\ = \frac{{( \begin{matrix}Biomarker spot protein \\ concentration per patient\end{matrix} )}^{**}}{{( \begin{matrix}Mean of normal biomarker spot \\ protein {concentrations}^{*}\end{matrix} )}^{**}}\end{matrix}$$

$${}^{*} Separately for Prospective and Retrospective samples , \\ {}^{**} Preferentially using 2 D gel protein spot density ( PPM ) , or in$$

$$another embodiment , using another measure of protein$$

$$concentration , such as µ g biomarker protein \text{/} ml of blood$$

$$serum , e . g . by Elisa immunossay$$

In this embodiment of the invention, comparison of prospective and retrospective samples on a fold differential expression basis provides for consistent results, as illustrated in FIGS. 3B, 3C.

FIGS. 3B, 3C illustrates a comparison for the retrospective samples, wherein the pattern of differential expression is essentially unaltered when converted from protein concentration as 2D gel protein spot density (PPM, FIG. 3A) to differential expression as fold of average 2D gel protein spot density (FIGS. 3B, 3C).

As also illustrated in FIGS. 3B and 3C, when retrospective and prospective samples are separately placed on a differential expression (fold of average normal) basis, the normal means coincide at 1.0 fold, and the differential expression of the prospective samples is now consistent with and readable on the retrospective samples (FIG. 3A, compared to FIG. 3B, Table XXXIV).

Ductal Carcinoma In Situ Breast Cancer (DCIS Bc) Vs. Non-Ductal Carcinoma In-Situ Breast Cancer (Non-DCIS BC)

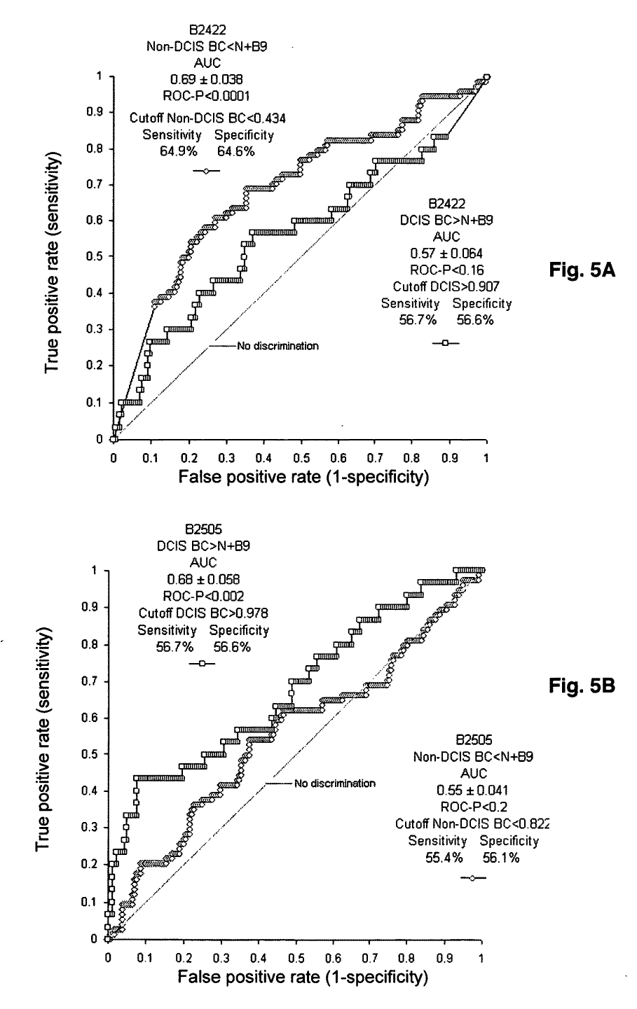

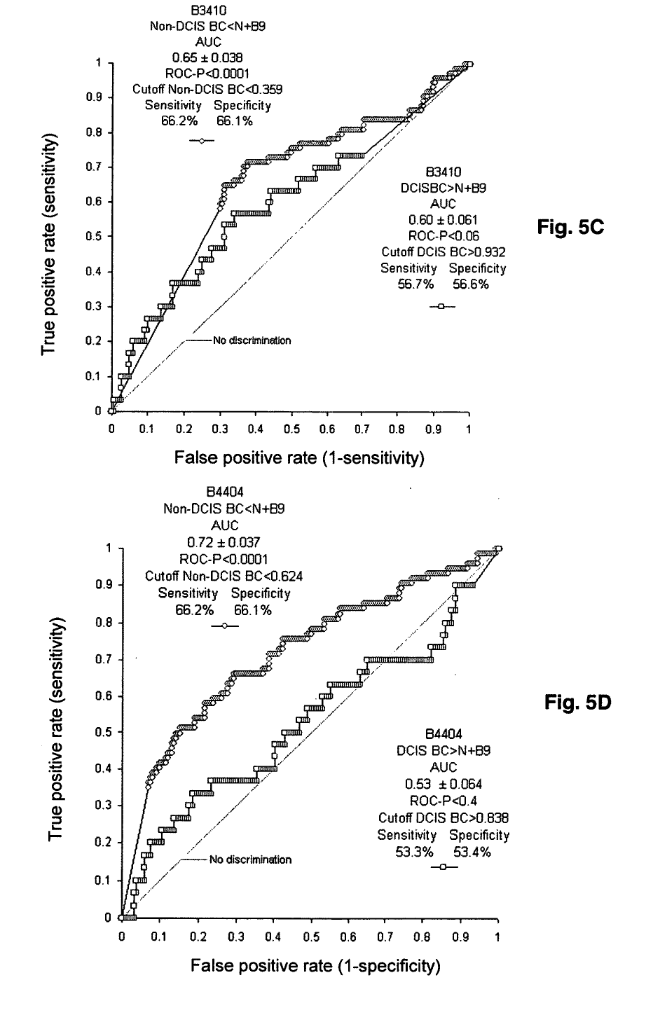

Illustrated in FIGS. 4A-4D and 5A-5D and Table XXXV a-d are the differential expression (in fold of average normal concentrations) of the individual biomarkers, the isoform spots of the 35 KD isoforms of the Inter-alpha-trypsin inhibitor heavy chain (H4) related protein biomarkers (FIGS. 4A, 5A: B2422, FIGS. 4A, 5B: B2505, FIGS. 4A, 5C: B3410, FIGS. 4A, 5D: B4404), wherein retrospective and prospective samples are combined after fold conversion. When these biomarkers are considered individually and earlier (DCIS BC) and later (Non-DCIS BC) stages of breast cancer are considered separately, isoform specific and stage specific differences in the differential expression from the normal controls are revealed. The non-DCIS breast cancer (Non-DCIS BC) concentrations are down-regulated, and the DCIS breast cancer (DCIS BC) concentrations are up-regulated in the blood serum of patients relative to the normal samples (FIGS. 4A-4D, 5A-5D, Table XXXV). Furthermore, the individual biomarker performance is not identical for each of the four isoforms, in that different degrees of up and/or down-regulation are found with statistically significant single variable biostatistics (FIGS. 4A-4D, 5A-5D, Table XXXV). This is illustrated by the less significant down-regulation of protein biomarker spot B2505 (FIG. 4B, FIG. 5B, Table XXXV b*) in non-DCIS breast cancer, relative to the other isoforms B2422, B3410, and B4404 ((FIGS. 4A, 4C, and 4D, Table XXXV a, c, d).

Thus, in a preferred embodiment of the invention, the blood serum concentrations of the different electrophoretic isoforms with the same protein amino acid sequence are nonetheless determined separately for greater diagnostic performance. Also in a preferred embodiment of the invention, DCIS, DCIS breast cancer, and non-DCIS breast cancer may be considered as separate groups for the purposes of the invention.

### Additional Protein Biomarkers

Additional spot identifications by LC MS/MS of in-gel trypsin digests, and pI and Molecular Weight estimations from 2D gels and amino acid sequences (FIGS. 6, 7 and Tables III-VI) indicated that:
Biomarker protein spot B1322 (FIG. 6) corresponds to an Immunoglobulin Lambda protein (Tables VI-VII, SEQ ID NO: 1); and
Biomarker protein spot B1418 (FIG. 6) corresponds to an Alpha-1-microglobulin protein (Tables VI and VIII, SEQ ID NO: 2); and
Biomarker protein spot B2317 (FIG. 6) corresponds to an Apolipoprotein A-1 protein (Tables VI, IX, SEQ ID NO: 3); and
Biomarker protein spot B3406 (FIG. 6) corresponds to an Apolipoprotein E3 protein (Tables VI, XIII, SEQ ID NO: 6); and
Biomarker protein spot B5539 (FIG. 6) corresponds to a human Albumin protein (Tables VI, XIV, SEQ ID NO: 7); and
Biomarker protein spot B6519 (FIG. 6) corresponds to a human Albumin protein (Tables VI, XV, SEQ ID NO: 8); and
Biomarker protein spot B6605 (FIG. 6) corresponds to a Transferrin protein (Tables VI, XVI, SEQ ID NO: 9); and
Biomarker protein spot B7408 (FIG. 6) corresponds to a Complement C4A gamma protein (Tables VI, XVII-XVIII, SEQ ID NO:_{—}10); and
Biomarker protein spots B1512, B4008, B4206, and B6014 (FIG. 6) correspond to electrophoretic isoforms of a Haptoglobin alpha chain and/or a Haptoglobin beta chain protein (Tables VI, XIX, SEQ ID NO: 11); and
Biomarker protein spots B3507, and B4424 (FIG. 6) correspond to electrophoretic isoforms of a Haptoglobin related protein (Tables VI, XX, SEQ ID NO: 12); and
Biomarker protein spots B2412 (FIG. 6) correspond to an Apoptosis Inhibitor protein (AIM) and/or a CD5L protein (Tables VI, XXI-XXII, SEQ ID NOS: 13-14); and
Biomarker protein spot B5713 (FIG. 6) corresponds to a Serotransferrin protein (Tables VI, XXIV, SEQ ID NO: 23); and
Biomarker protein spot B6218 (FIG. 6) corresponds to a Nucleolar and Ribosomal protein L27a protein (Tables VI, XXV-XXVII, SEQ ID NOS: 15-17); and
Biomarker protein spot B2412 (FIG. 6) corresponds to a Reticulon-4 protein (Tables VI, XXVIII-XXXII, SEQ ID NOS: 18-22).

As shown in FIGS. 8A-8B, the blood serum concentrations of Immunoglobulin lambda (λ) light chain biomarker protein spot B1322 (FIG. 8A, 8B, Table XXXVI) demonstrates a modest down shift in blood serum concentration, between that of normal controls (N) and that of both patients with benign breast disease or abnormalities (B9), and patients with Breast Cancer (BC).

As shown in FIGS. 9A-9B, the blood serum concentrations of Alpha-1-microglobulin biomarker protein spot B1418 (FIGS. 9A, 9B, Table XXXVII) demonstrates a modest and progressive up shift in blood serum concentration, from normal controls (N) to those of patients with benign breast disease or abnormalities (B9), and patients with Breast Cancer (Combined BC). The concentration appears to be maximal in DCIS BC (Table XXXVI).

As shown in FIGS. 10A-10B, Apolipoprotein A-I biomarker protein spot B2317, (FIGS. 10A, 10B, Table XXXVIII), demonstrates a down shift in blood serum concentration between normal controls (N) and patients with benign breast disease or abnormalities (B9), and conversely demonstrated an up-shift between normal controls (N) and patients with DCIS breast cancer (DCIS BC).

As shown in FIGS. 11A-11B, Apolipoprotein E3 biomarker protein spot B3406 (FIGS. 11A, 11B, Table XXXIX), demonstrates an down shift in blood serum concentration between normal controls (N) and patients with benign breast disease or abnormalities (B9), and conversely demonstrated an up-shift between patients with benign breast disease or abnormalities (B9) and patients with DCIS breast cancer (DCIS BC), and a corresponding return to normal levels in patients with Non-DCIS breast cancer (Non-DCIS BC).

As shown in FIGS. 12A-12B, Serum albumin biomarker protein spot B5539 (FIGS. 14 A, B, Table XL) demonstrated an up-shift in blood serum concentration between normal controls (N) and patients with benign breast disease or abnormalities (B9), and conversely demonstrated a progressive down-shift between patients with benign breast disease or abnormalities (B9), patients with DCIS breast cancer (DCIS BC), and patients with Non-DCIS breast cancer (Non-DCIS BC) to levels below normal (N).

As shown in FIGS. 13A-13B, Lectin P35 biomarker protein spot B6519 (FIGS. 13A, 13B, Table XLIX) demonstrated a progressive up shift in blood serum concentration from that of normal controls (N) and patients with benign breast disease or abnormalities (B9), to that of patients with DCIS breast cancer (DCIS BC), and patients with Non-DCIS breast cancer (Non-DCIS BC). As shown in FIGS. 14A-14B, Transferrin biomarker protein spot B6605 (FIGS. 14A, 14B, Table XLI) demonstrated an up-shift in blood serum concentration between that of normal controls (N) and that of patients with benign breast disease or abnormalities (B9), patients with DCIS breast cancer (DCIS BC), and patients with Non-DCIS breast cancer (Non-DCIS BC). The effect appeared to be maximal in patients with benign breast disease or abnormalities (B9) and to be progressively lower in patients with DCIS breast cancer (DCIS BC) and Non-DCIS breast cancer (Non-DCIS BC).

As shown in FIGS. 15A-15B, Complement C4A biomarker protein spot B7408 (FIGS. 15A, 15B, Table L) demonstrated an up-shift in blood serum concentration between that of normal controls (N) and that of patients with benign breast disease or abnormalities (B9), patients with DCIS breast cancer (DCIS BC), and patients with Non-DCIS breast cancer (Non-DCIS BC). The effect appeared to be maximal in patients with DCIS breast cancer (DCIS BC).

As shown in FIGS. 16A-16D, Haptoglobin biomarker protein spot B1512 (FIGS. 16A-16D, Table XLIII) a progressive up shift in blood serum concentration from that of normal controls (N) to that of patients with benign breast disease or abnormalities (B9), to that of patients with DCIS breast cancer (DCIS BC), and maximally to that of patients with Non-DCIS breast cancer (Non-DCIS BC).

As shown in FIGS. 17A-17B, Apoptosis Inhibitor (AIM and/or CD5L) biomarker protein spot B2412 (FIGS. 17A, 17B, Table LI) demonstrated a progressive up-shift in blood serum concentration between that of normal controls (N) and that of patients with benign breast disease or abnormalities (B9), patients with DCIS breast cancer (DCIS BC), and patients with Non-DCIS breast cancer (Non-DCIS BC). The effect appeared to be maximal in patients with DCIS breast cancer (DCIS BC).

As shown in FIGS. 18A-18B, Haptoglobin biomarker protein spot B4008 (FIGS. 18A, 18B, Table XLV) demonstrated an up-shift in blood serum concentration from that of normal controls (N) to that of patients with benign breast disease or abnormalities (B9), patients with DCIS breast cancer (DCIS BC), and patients with Non-DCIS breast cancer (Non-DCIS BC).

As shown in FIGS. 19A-19B, Haptoglobin biomarker protein spot B4206 (FIGS. 19A, 19B, Table XLVI) demonstrated an up-shift in blood serum concentration from that of normal controls (N) to that of patients with benign breast disease or abnormalities (B9), patients with DCIS breast cancer (DCIS BC), and patients with Non-DCIS breast cancer (Non-DCIS BC). The effect appeared to be maximal in patients with DCIS breast cancer (DCIS BC).

As shown in FIGS. 20A-20B, Haptoglobin related biomarker protein spot B4424 (FIGS. 20A, 20B, Table XLVIII) demonstrated a slight down-shift in blood serum concentration from that of normal controls (N) and patients with benign breast disease or abnormalities (B9), to that of patients with DCIS breast cancer (DCIS BC), and patients with Non-DCIS breast cancer (Non-DCIS BC). The effect appeared to be slightly more pronounced in that of patients with DCIS breast cancer (DCIS BC) than that of patients with Non-DCIS breast cancer (Non-DCIS BC).

As shown in FIGS. 21A-21B, Haptoglobin related biomarker protein spot B3506 (FIGS. 21A, 21B, Table XLVII) demonstrated a slight down-shift in blood serum concentration from that of normal controls (N) to that of patients with benign breast disease or abnormalities (B9), patients with DCIS breast cancer (DCIS BC), and patients with Non-DCIS breast cancer (Non-DCIS BC).

As shown in FIGS. 22A-22B, Serotransferrin biomarker protein spot B5713 (FIGS. 22A, 22B, Table XLII) demonstrated a down-shift in blood serum concentration from that of normal controls (N) to that of patients with benign breast disease or abnormalities (B9), patients with DCIS breast cancer (DCIS BC), and patients with Non-DCIS breast cancer (Non-DCIS BC).

As shown in FIGS. 23A-23C, Haptoglobin biomarker protein spot B6014 (FIGS. 23A, 23B, 23C, Table XLIV) demonstrated differential expression wherein a greater number (62.2%) of samples contained detectable blood serum levels of this biomarker in Non-DCIS breast cancer (Non-DCIS BC), than in normal controls and patients with benign breast disease or abnormalities (N+B9, 32.3%) and in patients with DCIS breast Cancer (DCIS BC).

As shown in FIGS. 24A-24B, Nucleolar and/or Ribosomal protein L27a biomarker protein spot B6218 (FIGS. 24A, 24B, Table LII) demonstrated an up-shift in blood serum concentration from that of normal controls (N) and patients with benign breast disease or abnormalities (B9), to that of patients with DCIS breast cancer (DCIS BC), and patients with Non-DCIS breast cancer (Non-DCIS BC). The effect appeared to be maximal in patients with DCIS breast cancer (DCIS BC).

As shown in FIGS. 25A-25D, Nucleolar and/or Reticulon-4 biomarker protein spot B7108 (FIGS. 25A-25D, Table LIII) demonstrated a progressive down-shift in blood serum concentration from that of normal controls (N) to that of patients with benign breast disease or abnormalities (B9), to that of patients with DCIS breast cancer (DCIS BC), and most pronounced in that of patients with Non-DCIS breast cancer (Non-DCIS BC).

While individual single variable non-parametric statistics of each of the 22 protein biomarkers in blood serum indicated significant disease specific differential expression, no single biomarker was capable of fully distinguishing between all the normal samples, benign samples, and breast cancer samples. However, the individual biomarkers performed differently from one another and when used together, employing multivariate linear discriminant analysis (Table X), the 22 biomarkers employed as a group were capable of synergistic discrimination of the three groups from each other (3-way, A & B) and between cancer and not cancer (2 way, C & D) with higher sensitivities and specificities (Table LIV). Furthermore, a group of 9 biomarkers selected by the Step Disc function of the linear discriminant analysis was essentially as good as the entire group of 22 biomarkers (Table LIV, compare a and b). As shown in FIGS. 26A-26B, (FIGS. 26A, 26B, Table LIV), the median differential expression profiles (median fold of mean normal blood serum concentration, where concentration=median 2D gel spot density, PPM) showed distinct differences between normal controls (Normal Median), patients with benign breast disease or abnormalities (B9 Median), patients with DCIS breast cancer (DCIS BC Median), and patients with Non-DCIS breast cancer (Non-DCIS BC Median). Furthermore, when these profiles are displayed in order of selection by the Step Disk function (FIG. 26A), a pattern is revealed wherein:
Apolipoprotein A-1 biomarker protein spot B2317 preferentially separates DCIS-BC from N+B9+Non-DCIS BC; followed by
ITI (H4) RP 35 KD protein isoform biomarker protein spot B2505, which preferentially separates DCIS-BC and to a lesser extent Non-DCIS BC from N+B9; followed by
Nucleolar and/or Ribosomal protein L27a biomarker protein spot B6218, which preferentially separates DCIS-BC and Non-DCIS BC from N+B9; followed by
Haptoglobin biomarker protein spot B6014, which preferentially separates Non-DCIS BC from N+B9+DCIS BC; followed by
Haptoglobin biomarker protein spot B1512, which preferentially separates Non-DCIS BC from N+B9+DCIS BC; followed by
Reticulon-4 biomarker protein spot B7108, which preferentially separates Non-DCIS BC from N+B9+DCIS BC; followed by
Serum Albumin protein spot B5539, which preferentially separates Non-DCIS BC from N+B9+DCIS BC; followed by
ITI (H4) RP 35 KD protein isoform biomarker protein spot B2422, which preferentially separates DCIS-BC and Non-DCIS BC from N+B9; followed by
ITI (H4) RP 35 KD protein isoform biomarker protein spot B2422, which preferentially separates DCIS-BC from N+B9+Non-DCIS BC.

The aforementioned Step Disc series of biomarkers (below the arrow, FIG. 26A) outlines how each new biomarker is synergistic with the previously selected biomarkers, arriving at the utility of specificity and sensitivity of the multivariate biostatistical analysis of the invention.

The additional 13 of the 22 biomarkers not selected by the Step Disc function are also displayed (below the dotted line, FIG. 26A) which also show distinct differences in separation between the groups of patients and controls. However, Based upon the slight increases in sensitivities and specificities obtained when they are also employed in the multivariate analysis (Table LIV b), these differences are largely redundant with the other nine biomarkers.

FIG. 26B further illustrates this redundancy when the individual isoforms are displayed in the order that they were selected into the Step Disk function, wherein:
Step Disk selected ITI (H4) RP 35 KD isoform spots B2505, B2422, and B4404, but not isoform spot B3410; and wherein
Step Disk selected Haptoglobin isoform spots B6014 and B1512, but not isoform spots B4008 nor B4206; and wherein
Step Disk selected neither Haptoglobin related protein isoform spots B3506 nor B4424.

On the other hand, when additional patient samples are added to the database, these additional “redundant” biomarkers provide further synergy to the invention.

The serum samples may also be subjected to various other techniques known in the art for separating and quantitating proteins. Such techniques include, but are not limited to gel filtration chromatography, ion exchange chromatography, reverse phase chromatography, affinity chromatography (typically in an HPLC or FPLC apparatus), or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture either as a one-dimensional, two-dimensional, three-dimensional or multi-dimensional protein pattern, and or the pattern of protein post synthetic modification isoforms.

Quantitation of a protein by antibodies directed against that protein is well known in the field. The techniques and methodologies for the production of one or more antibodies to the proteins, routine in the field and are not described in detail herein.

As used herein, the term antibody is intended to refer broadly to any immunologic binding agent such as IgG, 1gM, IgA, IgD and IgE. Generally, IgG and/or 1gM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit and even chicken origin. Due to the ease of preparation and ready availability of reagents, murine monoclonal antibodies are generally preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has 20 an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the one or more of the 22 protein biomarkers may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include microarrays of antibodies or proteins contained on a glass slide or a silicon chip, for example.

It is contemplated that arrays of antibodies to up to 22 protein biomarkers, or peptides derived, may be produced in an array and contacted with the serum samples or protein fractions of serum samples in order to quantitate the proteins. The use of such microarrays is well known in the art and is described, for example in U.S. Pat. No. 5,143,854, incorporated herein by reference.

The present invention includes a screening assay for breast cancer based on the up-regulation and/or down-regulation of the 22 protein biomarkers. One embodiment of the assay will be constructed with antibodies recognizing up to 22 protein biomarkers. One or more antibodies targeted to antigenic determinants of up to 22 protein biomarkers will be spotted onto a surface, such as a polyvinyl membrane or glass slide. As the antibodies used will each recognize an antigenic determinant of up to 22 protein biomarkers, incubation of the spots with patient samples will permit attachment of up to 22 protein biomarkers to the antibody.

The binding of up to 22 protein biomarkers can be reported using any of the known reporter techniques including radioimunoassays (RIA), stains, enzyme linked immunosorbant assays (ELISA), sandwich ELISAs with a horseradish peroxidase (HRP)-conjugated second antibody also recognizing up to 22 protein biomarkers, the pre-binding of fluorescent dyes to the proteins in the sample, or biotinylafing the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

As shown in Table X, the N-terminal of the of ITI (H4) RP PK-120 precursor is different from the ITI (H4) RP 35 KD isoforms, wherein the sequence containing the 35 KD (PK-120), corresponds to biomarkers B2422, B2595, B3410, and B4404 of the present invention is located in the C-terminal sequence. The lack of homology is maintained throughout the 35 KD product. For high throughput immunoassays, biomarker specific antibodies can be developed using truncated cDNA sequences to produce recombinant antigens in bacterial or mammalian systems, containing only the epitopes of the 35 KD biomarkers without the epitopes of the upstream region of the parent molecules. These antigens in turn can be used to immunize rabbits, sheep, chickens, or goats, for polyclonal antibodies, or mice to produce monoclonal antibodies either with classic hybridoma technologies or phage display methods. The recombinant antigens can also be employed as affinity agents to purify antibodies and as reagent controls in assays.

Alternatively, antibodies could be raised to the upstream portions of the parent molecule that would not cross react with the ITI (H4) RP 35 KD isoforms (Table X). Such antibodies could be used as affinity capture agents to isolate from serum or other sources the intact PK 120. Subsequent treatment of this group with plasma Kallikrein, which selectively cleaves out the ITI (H4) RP 35 KD isoforms would release the 35 KD isoforms, which would not bind the antibodies and thus the biomarkers, in native purified form, can be obtained from a biological sample.

Similar approaches are available for the other of up to 22 biomarkers whose amino acid sequences are defined in some of the accompanying tables.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention.

More specifically, it is well recognized in the art that the statistical data, including but not limited to the mean, standard error, standard deviation, median, interquartile range, 95% confidence limits, results of analysis of variance, non-parametric median tests, discriminant analysis, etc., will vary as data from additional patients are added to the database or antibodies are utilized to determine concentrations of one or more of the 22 biomarkers of the present invention, or any biomarker. Therefore changes in the statistical values of one or more of the 22 protein biomarkers do not depart from the concept, spirit and scope of the invention.

Also more specifically, it is disclosed (in cross referenced U.S. Utility patent applications by Goldknopf, I. L., et al. Ser. Nos. 11/507,337 and 11/503,881, U.S. Provisional Patent Applications by Goldknopf et al. Ser. No. 60/708,992 and 60/738,710, and referenced in Goldknopf, I. L et al. 2006 and Sheta et al. 2006, hereby incorporated as reference) that blood serum concentrations of protein biomarkers, including an inter alpha trypsin inhibitor family heavy chain (H4) related protein 35 KD and Apolipoprotein E3, can be used in combination with other biomarkers for diagnosis, differential diagnosis, and screening. Consequently, the use of one or more of the 22 protein biomarkers in conjunction with one or more additional biomarkers not disclosed in the present invention does not depart from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Tables I-LV

TABLE I
Table I: Staging Breast Cancer

| | | Lymph Node | Metastasis* | |
|---|---|---|---|---|
| Stage | Tumor Size | Involvement | (Spread) | AKA |
| 0 | In situ | No | No | Carcinoma |
| | (DCIS, LCIS) | | | in situ |
| I | Less than 2 cm | No | No | Invasive |
| II | Between 2-5 cm | No or in same side | No | carcinoma |
| | | of breast | | |
| III | More than 5 cm | Yes, on same side | Yes | |
| | | of breast | | |
| IV | Not applicable | Not applicable | | |

*No = not detected

TABLE II

| Protein | pI | Molecular Weight (Da) |
|---|---|---|
| Hen egg white conalbumin | 6.0, 6.3, 6.6 | 76,000 |
| Bovine serum albumin | 5.4, 5.5, 5.6 | 66,200 |
| Bovine muscle actin | 5.0, 5.1 | 43,000 |
| Rabbit muscle GAPDH | 8.3, 8.5 | 36,000 |
| Bovine carbonic anhydrase | 5.9, 6.0 | 31,000 |
| Soybean trypsin inhibitor | 4.5 | 21,500 |
| Equine myoglobin conalbumin | 7.0 | 17,500 |

TABLE III

| | Relative Mobility (Rf) | |
|---|---|---|
| | (Fold of 10,000 MW | |
| Biomarker Spot | distance from origin) | y = 13043x − 1.0128 |
| B1322 | 0.604 | 21,738 |
| B1418 | 0.474 | 27,780 |
| B2713 | 0.630 | 20,830 |
| B2422 | 0.448 | 29,411 |
| B2505 | 0.429 | 30,766 |
| B3410 | 0.468 | 28,170 |
| B4404 | 0.487 | 27,029 |
| B3406 | 0.442 | 29,849 |
| B5539 | 0.325 | 40,755 |
| B6519 | 0.422 | 31,245 |
| B6605 | 0.253 | 52,417 |
| B7408 | 0.461 | 28,572 |
| B1512 | 0.325 | 40,755 |
| B2412 | 0.506 | 25,977 |
| B4008 | 1.091 | 11,943 |
| B4206 | 0.740 | 17,687 |
| B3507 | 0.396 | 33,321 |
| B4424 | 0.403 | 32,777 |
| B5713 | 0.169 | 79,034 |
| B6014 | 0.896 | 14,576 |
| B6218 | 0.792 | 16,513 |
| B7108 | 0.948 | 13,767 |

TABLE IV
Calculation of pI From 2D Gel Electrophoresis

| Spot | Relative Focusing | pI |
|---|---|---|
| Acidic end | 0.0000 | 5.00 |
| Basic end | 1.0000 | 8.00 |
| B1322 | 0.0875 | 5.26 |
| B1418 | 0.0798 | 5.24 |
| B2317 | 0.1787 | 5.54 |
| B2422 | 0.2015 | 5.60 |
| B2505 | 0.1445 | 5.43 |
| B3410 | 0.2700 | 5.81 |
| B4404 | 0.3536 | 6.06 |
| B3406 | 0.2510 | 5.75 |
| B5539 | 0.4715 | 6.41 |
| B6519 | 0.6464 | 6.94 |
| B6605 | 0.6008 | 6.80 |
| B7408 | 0.7338 | 7.20 |
| B1512 | 0.0760 | 5.23 |
| B2412 | 0.1293 | 5.39 |
| B4008 | 0.3574 | 6.07 |
| B4206 | 0.3422 | 6.03 |
| B3506 | 0.2852 | 5.86 |
| B4424 | 0.4639 | 6.39 |
| B5713 | 0.5513 | 6.65 |
| B6014 | 0.6768 | 7.03 |
| B6218 | 0.6768 | 7.03 |
| B7108 | 0.7452 | 7.24 |

TABLE V

| | |
|---|---|
| 2D Gel | Amino Acid Sequence |

| | MW | pI | MW | pI |
|---|---|---|---|---|
| B1322 | 21,738 | 5.26 | 24,489 | 5.8 |
| B1416 | 27,780 | 5.24 | 20,433 | 5.8 |
| B2713 | 20,830 | 5.54 | 28,962 | 5.4 |
| B2422 | 29,411 | 5.60 | 26,970 | 6.3 |
| | | | 28,253 | 7.1 |
| B2505 | 30,766 | 5.43 | 26,970 | 6.3 |
| | | | 28,253 | 7.1 |
| B3410 | 28,170 | 5.81 | 26,970 | 6.3 |
| | | | 28,253 | 7.1 |
| B4404 | 27,029 | 6.06 | 26,970 | 6.3 |
| | | | 28,253 | 7.1 |
| B3406 | 29,849 | 5.75 | 34,364 | 5.5 |
| B5539 | 40,755 | 6.41 | 44,994 | 5.9 |
| B6519 | 31,245 | 6.94 | 34,019 | 6.1 |
| B6605 | 52,417 | 6.80 | 77,051 | 6.8 |
| B7408 | 28,572 | 7.20 | 33,074 | 6.4 |
| B1512 | 40,755 | 5.39 | 45,206 | 6.1 |
| B2412 | 25,977 | 5.42 | 38,088 | 5.3 |
| | | | 38,130 | 5.3 |
| B4008 | 11,943 | 6.07 | 45,206 | 6.1 |
| B4206 | 17,687 | 6.03 | 45,206 | 6.1 |
| B3507 | 33,321 | 5.86 | 39,008 | 6.4 |
| B4424 | 32,777 | 6.39 | 39,008 | 6.4 |
| B5713 | 79,034 | 6.65 | 77,051 | 6.8 |
| B6014 | 14,576 | 7.03 | 45,206 | 6.1 |
| B6218 | 16,513 | 7.03 | 16,430 | 11.0 |
| | | | 16,044 | 10.7 |
| B7108 | 13,767 | 7.24 | 12,932 | 4.8 |

TABLE VI

| Spot # | Protein ID | Accession # | # of peptides | Sequence # |
|---|---|---|---|---|
| B1332 | Immunoglobulin lambda chain | 106653 | 2 | 1 |
| B1418 | Alpha-1-microglobulin | 223373 | 3 | 2 |
| B2317 | Apolipoprotein A1 | 178775 | 9 | 3 |
| B2422 | Inter-α-trypsin inhibitor family heavy chain | 1483187 | 5 | 4, 5 |
| | related protein (ITIHRP) | | | |
| B2505 | Inter-α-trypsin inhibitor family heavy chain | 1483187 | 3 | 4, 5 |
| | related protein (ITIHRP) | | | |
| B3406 | Apolipoprotein E3 | 178849 | 3 | 6 |
| | | 1942471 | 4 | |
| B3410 | Inter-α-trypsin inhibitor family heavy chain | 1483187 | 4 | 4, 5 |
| | related protein (ITIHRP) | | | |
| B4404 | Inter-α-trypsin inhibitor family heavy chain | 1402590 | 3 | 4, 5 |
| | related protein (ITIHRP) | | | |
| B5539 | Serum Albumin Protein | 28590 | 5 | 7 |
| B6519 | Lectin P35 3 | 1669349 | 3 | 8 |
| B6605 | Transferrin | 4557871 | 9 | 9 |
| B7408 | Complement component C4A | 179674 | 2 | 10 |
| B1512 | Haptoglobin precursor[Contains: Haptoglobin | P00738 | 24 | 11 |
| | alpha chain; Haptoglobin beta chain] | | | |
| B2412 | Apoptosis inhibitor expressed by Macrophages | 4102235 | 9 | 13, 14 |
| | Human secreted protein CD5L [Homo sapiens] | 37182111 | | |
| B4008 | Haptoglobin precursor [Contains: Haptoglobin | P00738 | 9 | 11 |
| | alpha chain; Haptoglobin beta chain] | | | |
| B4206 | Haptoglobin precursor [Contains: Haptoglobin | P00738 | 11 | 11 |
| | alpha chain; Haptoglobin beta chain] | | | |
| B3506 | Haptoglobin-related protein precursor | P00739 | 8 | 12 |
| B4424 | Haptoglobin-related protein precursor | P00739 | 5 | 12 |
| B5713 | Serotransferrin precursor (Transferrin) | P02787 | 6 | 23 |
| | (Siderophilin) (Beta-1-metal-binding globulin) | | | |
| B6014 | Haptoglobin precursor [Contains: Haptoglobin | P00738 | 10 | 11 |
| | alpha chain; Haptoglobin beta chain] | | | |
| B6218 | Unknown (protein for IMAGE: 3543815) [60S | 18042923 | 1 | 15-17 |
| | ribosomal protein L27a] | | | |
| B7108 | Reticulon-4 (Neurite outgrowth inhibitor) | Q9NQC3 | 1 | 18-22 |
| | (Nogo protein) (Foocen) (Neuroendocrine- | | | |
| | specific protein) (NSP) (Neuroendocrine- | | | |
| | specific protein C homolog) (RTN-x) | | | |
| | (Reticulon-5) - Homo sapiens (Human) | | | |

TABLE VII

span of LC/MS/MS identified peptides underlined
MAWTVLLLGL LSHCTGSVTS YVLTQPPSVS VAPGKTASIT
CGGNNIGSKS VHWYQQKPGQ APVLVVYDDS DRPSGIPERF
SGSNSGNTAT LTISRVEAGD EADYYCQVWD SSSDVVFGGG
TKLTVLGQPK AAPSVTLFPP SSEELQANKA TLVCLISDFY
PGAVTVAWKA DSSPVKAGVE TTTPSKQSNN KYAASSYLSL
TPEQWKSHRS YSCQVTHEGS TVEKTVAPTE CS
(SEQ ID NO: 1)

pI of Protein: 5.8
Protein MW: 24489
Accession #106653

TABLE VIII

GPVPTPPDNI QVQENFNISR IYGKWYNLAI GSTCPLKIMD RMTVSTLVLG EGATEAEISM TSTRWRKGVC
EETSGAYEKT DTDGKFLYHK SKWNITMESY VVHTNYDEYA IFLTKKFSRH HGPTITAKLY GRAPQLRETL
LQDFRVVAQG VGIPEDSIFT MADRGECVPG EQEPEPILIP R
(SEO ID NO: 2)
MS-Digest Search Results: span of LC/MS/MS identified peptides underlinedpI of Protein: 5.8
Protein MW: 20433
Accession #223373
Protein alternative names:
HCP; IATIL; ITIL; OTTHUMP00000063975; UTI
ALPHA-1 MICROGLOBULIN/BIKUNIN PRECURSOR
Alpha-1 -microglobulin/bikunin precursor (inter-alpha-trypsin inhibitor, light chain; protein HC)
Alpha--microglobulin/bikunin precursor; inter-alpha-tiypsin
COMPLEX-FORMING GLYCOPROTEIN HETEROGENEOUS IN CHARGE INTER-ALPHA-TRYPSIN INHIBITOR
The alpha-1-microglobulin (Protein HC) is a 31-kD, single chain plasma glycoprotein, which appears to
be involved in regulation of the inflammatory process (Mendez et al., 1986). The alpha-1-microglobulin/
bikunin precursor gene (AMBP) codes for a precursor that splits into alpha-1-microglobulin, which
belongs to the lipocalin superfamily, and bikunin (formerly HI-30, urinary trypsin inhibitor, inhibitor
subunit of inter-alpha-trypsin inhibitor). The amino acid sequence of the parental protein is provided
below:
Parental precursor protein alternative names:
Alpha-1-microglobulin (Protein HC) (Complex-forming glycoprotein heterogeneous in charge)/Inter-alpha-trypsin inhibitor light chain
(ITI-LC) (Bikunin) (HI-30)J complex
Parental protein sequence: span of LC/MS/MS identified peptides underlined:
Signal peptide (italics):

| | | |
|---|---|---|
| MRSLGALLL LSACLAVSAG PVPTPPDNIQ VQENFNISRI YGKWYNLAIG STCPWLKKIM | 60 | Alpha-1-microglobulin |
| DRMTVSTLVL GEGATEAEIS MTSTRWRKGV CEETSGAYEK TDTDGKFLYH KSKWNITMES | 120 | (bold letters) |
| YVVHTNYDEY AIFLTKKFSR HHGPTITAKL YGRAPQLRET LLQDFRVVAQ GVGIPEDSIF | 180 | |
| TMADRGECVP GEQEPEPILI PRVRRAVLPQ EEEGSGGGQL VTEVTKKEDS CQLGYSAGPC | 240 | Inter-α-Trypsin Inhibitor |
| MGMTSRYFYN GTSMACETFQ YGGCMGNGNN FVTEKECLQT CRTVAACNLP IVRGPCRAFI | 300 | light chain (Bikumin) |
| QLWAFDAVKG KCVLFPYGGC QGNGNKFYSE KECREYCGVP GDGDEELLRF SN | 352 | |
| (SEQ ID NO: 24) | | |

TABLE IX

| | |
|---|---|
| Protein alternative names: | |
| Amyloidosis | |
| APOLIPOPROTEIN OF HIGH DENSITY LIPOPROTEIN APOA1/APOC3 FUSION GENE | |
| Apolipoprotein A-I | |
| Apolipoprotein A-I precursor | |
| Proapolipoprotein | |
| Parental Protein Full Sequence: NCBI accession # 178775: | |
| Span of LC/MS/MS identified peptides underlined: | |
| 1 RHFWQQDEPP QSPWDRVKDL ATVYVDVLKD SGRDYVSQFE GSALGKQLNL KLLDNWDSVT | Sequence identical to |
| 61 STFSKLREQL GPVTQEFWDN LEKETEGLRQ EMSKDLEEVK AKVQPYLDDF QKKWQEEMEL | apolipoprotein Al lacking |
| 121 YRQKVEPLRA ELQEGARQKL HELQEKLSPL GEEMRDRARA HVDALRTHLA PYSDELRQRL | the n-terminal signal |
| 181 AARLEALKEN GGAELAEYHA KATEHLSTLS EKAKPALEDL RQGLLPVLES FKVSFLSALE | peptide [MKAAVLTLAVLFLTGSQA] |
| 241 EYTKKLNTQ | |
| (SEQ ID NO: 3) | |

MS-Digest Search Results
pI of Protein: 5.4
Protein MW: 28962

TABLE X

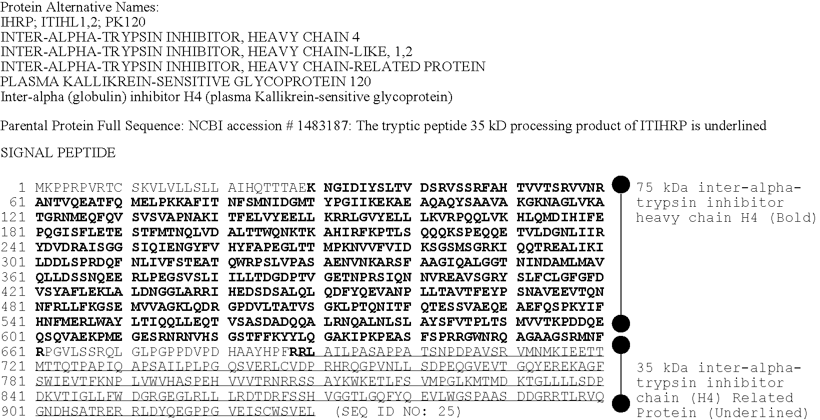

The amino acid sequence of the inter-alpha-trypsin inhibitor heavy chain (H4) related protein composed of 930 amino acids (Mwt 103.4 kDa). The N-terminal 28 residues corresponded to a signal peptide for secretion. The N-terminal 600 residues of the mature form exhibited considerable homology to those of Inter-alpha trypsin inhibitor (ITI) heavy chains, while the C-terminal 300 residues showed no homology with the heavy chains and low homology with ATP-dependent proteases. Inter-alpha-trypsin inhibitor heavy chain (H4) related protein is readily cleaved into 75- and 35-kDa fragments when plasma is incubated at 37 degrees C. The cleaved site, Arg-Arg-Leu (RRL), is within a proline-rich region (Saguchi et al, J Biochem (1995)117: 14-18). The 35-kDa cleavage fragment (underlined), expands the amino acid sequence starting at Arginine (R)-689 to Leucine (L)-930, is the fragment detected on 2D gel electrophoresis, marked as spots# 2422, 2505, 3410, and 4404 (Mwt 35 KD), it is most likely that the 4 protein spots corresponds to the 35 KD processing product in depicted in FIG. 1. [00510050] The sequence of peptides also exists in proteins with NCBI accession numbers: 1483187; 4096840; 7770149; 13432192; 55620443; 55732844, which belong to “Inter-alpha-trypsin inhibitor family heavy chain (H4) related protein family (ITIHRP; ITIH4).

TABLE XI

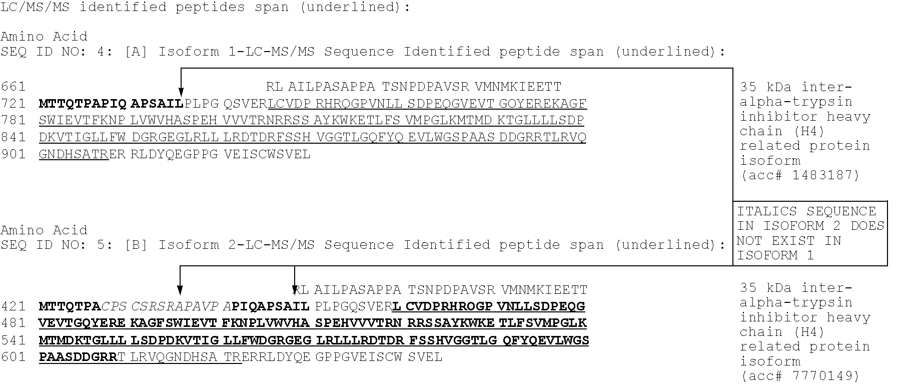

TABLE XII

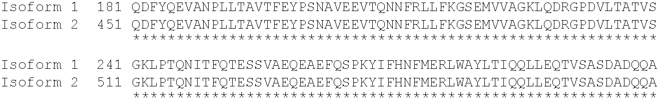

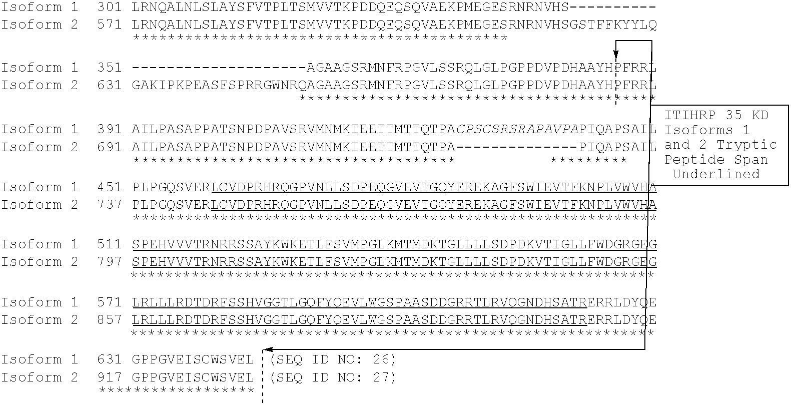

TABLE XIII

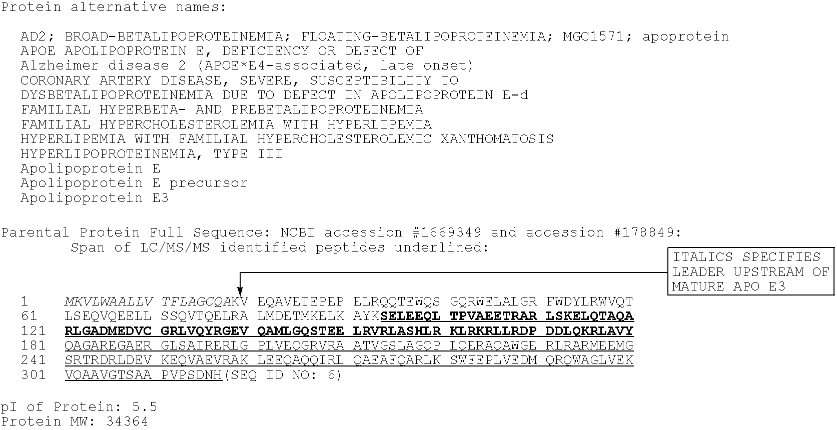

TABLE XIV

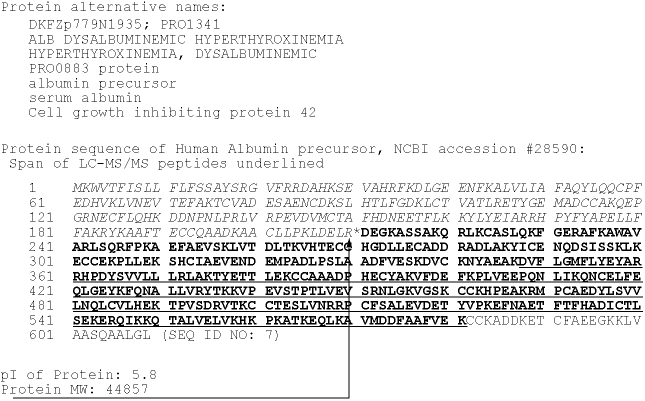

*Protein sequence that corresponds to spot B5539 has an estimated molecular weight of ~ 45 kD and pI of ~ 6.2, which is calculated to correspond to albumin fragment sequence that starts at Aspartic acid (D) residue number 211* extends to the C-terminal Leucine (L) residue # 609 and expands the LC-MS/MS identified peptides (underlined).

TABLE XV

```
Protein alternative names:
Ficolin-2 precursor (Collagen/fibrinogen domain-containing protein 2)
(Ficolin-B) (Ficolin B) (Serum Lectin p35) (EBP-37) (Heckling)
(L- Ficolin).
Parental Protein Full Sequence: NCBI accession #1669349:
Span op LC/MS/MS identified peptides underlined:
    1 MELDRAVGVL GAATLLLSFL GMAWALQAAD TCPEVKMVGL EGSDKLTILR GCPGLPGAPG
  61 DKGEAGTNGK RGERGPPGPP GKAGPPGPNGAPGEPQPCLT GPRTCKDLLD RGHFLSGWHT
121 IYLPDCRPLT VLCDMDTDGG GWTVFQRRVD GSVDFYRDWA TYKQGFGSRL GEFWLGNDNI
181 HALTAQGTSE LRVDLVDFED NYQFAKYRSF KVADEAEKYN LVLGAFVEGS AGDSLTFHNN
241 QSFSTKDQDN DLNTGNCAVM FQGAWWYKNC HVSNLNGRYL RGTHGSFANG INWKSGKGYN
301 YSYKVSEMKV RPA
(SEQ ID NO: 8)
```

TABLE XVI

```
Span of LC/MS/MS identified peptides underlined
Protein Sequence: NCBI Accession #4557871
    1 MRLAVGALLV CAVLGLCLAV PDKTVRWCAV SEHEATKCQS FRDHMKSVIP SDGPSVACVK
  61 KASYLDCIRA IAANEADAVT LDAGLVYDAY LAPNNLKPVV AEFYGSKEDP QTFYYAVAVV
121 KKDSGFQMNQ LRGKKSCHTG LGRSAGWNIP IGLLYCDLPE PRKPLEKAVA NFFSGSCAPC
181 ADGTDFPQLC QLCPGCGCST LNQYFGYSGA FKCLKDGAGD VAFVKHSTIF ENLANKADRD
241 QYELLCLDNT RKPVDEYKDC HLAQVPSHTV VARSMGGKED LIWELLNQAQ EHFGKDKSKE
301 FQLFSSPHGK DLLFKDSAHG FLKVPPRMDA KMYLGYEYVT AIRNLREGTC PEAPTDECKP
361 VKWCALSHHE RLKCDEWSVN SVGKIECVSA ETTEDCIAKI MNGEADAMSL DGGFVYIAGK
421 CGLVPVLAEN YNKSDNCEDT PEAGYFAVAV VKKSASDLTW DNLKGKKSCH TAVGRTAGWN
481 IPMGLLYNKI NHCRFDEFFS EGCAPGSKKD SSLCKLCMGS GLNLCEPNNK EGYYGYTGAF
541 RCLVEKGDVA FVKHQTVPQN TGGKNPDPWA KNLNEKDYEL LCLDGTRKPV EEYANCHLAR
601 APNHAVVTRK DKEACVHKIL RQQQHLFGSN VTDCSGNFCL FRSETKDLLF RDDTVCLAKL
661 HDRNTYEKYL GEEYVKAVGN LRKCSTSSLL EACTFRRP
(SEQ ID NO: 9)
```

pI of the Protein: 6.8
Molecular Weight: 77050 Da

TABLE XVII

Protein alternative names:
C4A2; C4A3; C4A4; C4A6; C4S; CO4
C4A anaphylatoxin
COMPLEMENT COMPONENT 4S
RODGERS FORM OF C4 COMPLEMENT COMPONENT 4A DEHCIENCY
acidic C4
c4 propeptide
complement component 4A preproprotein
complement component C4B
Span of LC/MS/MS Tryptic peptides underlined
1 11 21 31 41 51 61 71
EAPKVVEEQE SRVHYTVCIW RNGKVGLSGM AIADVTLLSG FHALRADLEK LTSLSDRYVS HFETEGPHVL LYFDSVPTSR
81 91 101 111 121 131 141 151
ECVGFEAVQE VPVGLVQPAS ATLYDYYNPE RRCSVFYGAP SKSRLLATLC SAEVCQCAEG KCPRQRRALE RGLQDEDGYR
161 171 181 191 201 211 221 231
MKFACYYPRV EYGFQVKVLR EDSRAAFRLF ETKITQVLHF TKDVKAAANQ MRNFLVRASC RLRLEPGKEY LIMGLDGATY
241 251 261 271 281 291
DLEGHPQYLL DSNSWIEEMP SERLCRSTRQ RAACAQLNDF LQEYGTQGCQ V
(SEQ ID NO: 10)

pI of Protein: 6.4
Protein MW: 33074 Da

TABLE XVIII

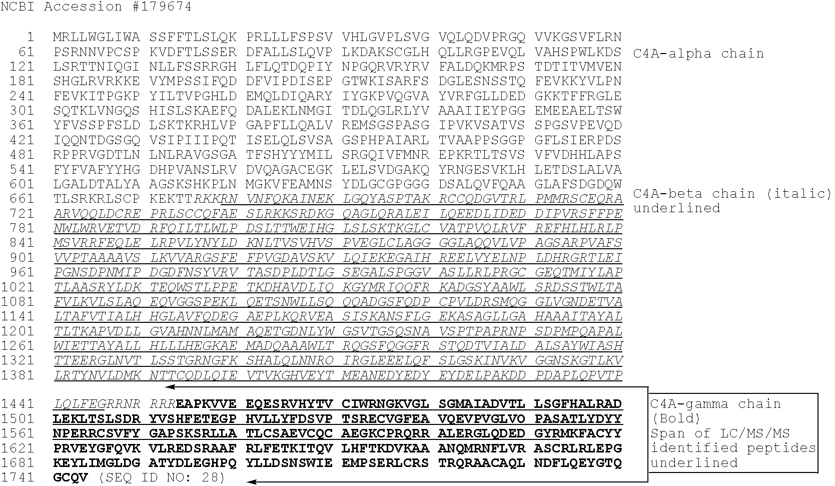

TABLE XIX
Accession #P00738.
Haptoglobin precu . . . [gi:123508]
Precursor Contains: Haptoglobin alpha chain:
Haptoglobin beta chain

(SEQ ID NO: 11)
MSALGAVIALLLWGQLFAVDSGNDVTDIADDGCPKPPEIAHGYVEHSVRY
QCKNYYKLRTEGDGVYTLNDKKQWINKAVGDKLPECEADDGCPKPPEIAH
GYVEHSVRYQCKNYYKLRTEGDGVYTLNNE KQWINKAVGDKLPECEAVC
GKPKNPANPVQRILGGHLDAKGSFPWQAKMVSHHNLTTGAT LINEQWLL
TTAKNLFLNHSENATAKDIAPTLTLYVGKKQLVEIEKVVLHPNYSQVDIG
LIKLKQKVSVNERVMPICLPSKDYAEVGRVGYVSGWGRNANFKFTDHLKY
VMLPVADQDQCIRHYEGSTVPEKKTPKSPVGVQPILNEHTFCAGMSKYQE
DTCYGDAGSAFAVHDLEEDTWYATGILSFDKSCAVAEYGVYVKVTSIQDW
VQKTIAEN
pI of Protein: 6.1
Protein MW: 45206
2D gel Results
B15 12: MW 38648;
B4008: MW 12257
B4206: MW 17699
B6014: MW 14768

TABLE XX
P00739. Haptoglobin-relat . . . [gi:123510]

(SEQ ID NO: 12)
MSDLGAVISLLLWGRQLFALYSGNDVTDISDDRFPKPPEIANGYVEHLFR
YQCKNYYRLRTEGDGVYTLNDKKQWINKAVGDKLPECEAVCGKPKNPANP
VQRILGGHLDAKGSFPWQAKMVSHHNLTTGATLINEQWLLTTAKNLFLNH
SENATAKDIAPTLTLYVGKKQLVEIEKVVLHPNYHQVDIGLIKLKQKVLV
NERVMPICLPSKNYAEVGRVGYVSGWGQSDNFKLTDHLKYVMLPVADQYD
CITHYEGSTCPKWKAPKSPVGVQPILNEHTFCVGMSKYQEDTCYGDAGSA
FAVHDLEEDTWYAAGILSFDKSCAVAEYGVYVKVTSIQDWVQKTIAEN
pI of Protein: 6.4
Protein MW: 39008
2D gel Results
B3606: MW 32011;
B4424: MW 31521

TABLE XXI
Peptides identified by LC
MS/MS of in-gel tryptic digests:

| | | |
|---|---|---|
| Accession | Sequence | Name |

| | | |
|---|---|---|
| gi\|4102235; | CSGEEQSLEQCQHR | AIM [Homo sapiens]; CDSL |
| gi\|37182111 | LVGGDNLCSGR | [Homo sapiens] |
| | IWLDNVR | |
| | CYGPGVGR | |
| | EATLQDCPSGPWGK | |
| | CSGEEQSLEQCQHR | |
| | HQNQWY | |
| | IWLDNVR | |
| | IWLDNVR | |

TABLE XXII
Accession #AAD01446 [gi:4102235]
Span of LCIMSIMS identified peptides underlined

(SEQ ID NO: 13)
MALLFSLILAICTRPGFLASPSGVRLVGGLHRCEGRVEVEQKGQWGTVCD
DGWDIKDVAVLCRELGCGAASGTPSGILYEPPAEKEQKVLIQSVSCTGTE
DTLAQCEQEEVYDCSHDEDAGASCENPESSFSPVPEGVRLADGPGHCKGR
VEVKHQNQWYTVCQTGWSLRAAKVVCRQLGCGRAVLTQKRCNKHAYGRKP
IWLSOMSCSGREATLQDCPSGPWGKNTCNHDEDTWVECEDPFDLRLVGGD
NLCSGRLEVLHKGVWGSVCDDNWGEKEDQVVCKQLGCGKSLSPSFRDRKC
YGPGVGRIWLDNVRCSGEEQSLEQCQHRFWGFHDCTHQEDVAVICSG
pI of Protein: 5.3
Protein MW: 38088
2D gel Results:
B2412: MW 25359

TABLE XXIII
Accession #AAQ88858. [gi:37182111];
Span of LC/MSIMS identified peptides underlined

(SEQ ID NO: 14)
MALLFSLILAICTRPGFLASPSGVRLVGGLHRCEGRVEVEQKGQWGTVCD
DGWDIKDVAVLCRELGCGAASGTPSGILYEPPAEKEQKVLIQSVSCTGTE
DTLAQCEQEEVYDCSHDEDAGASCENPESSFSPVPEGVRLADGPGHCKGR
VEVKHQNOWYTVCQTGWSLRAAKVVCRQLGCGRAVLTQKRCNKHAYGRKP
IWLSQMSCSGREATLQDCPSGPWGKNTCNHDEDTWVECEDPFDLRLVGGD
NLCSGRLEVLHKGVWGSVCDDNWGEKEDQVVCKQLGCGKSLSPSFRDRKC
YGPGVGRIWLDNVRCSGEEQSLEQCQHRFWGFHDCTHQEDVAVICSV
pI of Protein: 5.3
Protein MW: 38130
2D gel Results:
B2412: MW 25359

TABLE XXIV
(SEQ ID NO: 23)

MRLAVGALLV CAVLGLCLAV PDKTVRWCAV SEHEATKCQS
FRDHMKSVIP SDGPSVACVK KASYLDCIRA IAANEADAVT
LDAGLVYDAY LAPNNLKPVV AEFYGSKEDP QTFYYAVAVV
KKDSGFQMNQ LRGKKSCHTG LGRSAGWNIP IGLLYCDLPE
PRKPLEKAVA NFFSGSCAPC ADGTDFPQLC QLCPGCGCST
LNQYFGYSGA FKCLKDGAGD VAFVKHSTIF ENLANKADRD
QYELLCLDNT RKPVDEYKDC HLAQVPSHTV VARSMGGKED
LIWELLNQAQ EHFGKDKSKE FQLFSSPHGK DLLFKDSAHG
FLKVPPRMDA KMYLGYEYVT AIRNLREGTC PEAPTDECKP
VKWCALSHHE RLKCDEWSVN SVGKIECVSA ETTEDCIAKI
MNGEADAMSL DGGFVYIAGK CGLVPVLAEN YNKSDNCEDT
PEAGYFAVAV VKKSASDLTW DNLKGKKSCH TAVGRTAGWN
IPMGLLYNKI NHCRFDEFFS EGCAPGSKKD SSLCKLCMGS
GLNLCEPNNK EGYYGYTGAF RCLVEKGDVA FVKHQTVPQN
TGGKNPDPWA KNLNEKDYEL LCLDGTRKPV EEYANCHLAR
APNHAVVTRK DKEACVHKIL RQQQHLFGSN VTDCSGNFCL
FRSETKDLLF RDDTVCLAKL HDRNTYEKYL GEEYVKAVGN
LRKCSTSSLL EACTFRRP
pI of Protein: 6.8
Protein MW: 77051

TABLE XXV
Accession AAH20169. [gi:18042923]
Span of LC/MS/MS identified peptides underlined:

(SEQ ID NO: 15)
PSRLRKTRKLRGHVSHGHGRIGKHRKHPGGRGNAGGLHHHRINFDKYHPG
YFGKVGMKHYHLKRNQSFCPTVNLDKLWTLVSEQTRVNAAKNKTGAAPII
DVVRSGYYKVLGKGKLPKQPVIVKAKFFSRRAEEKIKSVGGACVLVA
gb|AAH2O169.1|AAH20169 Unknown (protein for
IMAGE:3543815) [Homo sapiens]
Length = 147
pI of Protein: 11.0
Protein MW: 16430

TABLE XXVI

Accession NP_000981 [gi:4506625]
Span of LC/MS/MS identified peptides underlined:

(SEQ ID NO: 16)
MPSRLRKTQKLRGHVSHGHGRIGKLQKHPRGHSNAGGMHHHRINFNKYYP
GYFGKVGMRYYLKRNQTVSLDKLWTLVSEQTQVNAAKNKPGAAPLIDVVQ
SGYYKVLGKEKLPKQPVIVKAKFFSRRAEKIKGVKGTCVLVA
ref|NP_000981.1| ribosomal protein L27a
[Homo sapiens]
sp| P46776| RL27A HUMAN 60S ribosomal protein L27a
gb| AAA85656.1| ribosomal protein L27a
dbj|BAA77361.1| ribosomal protein L27A
[Homo sapiens]
gb|AAH05326.1| Ribosomal protein L27a
[Homo sapiens]
gb|EAW68619.1| ribosomal protein L27a
[Homo sapiens]
prf|12113200C ribosomal protein L27a
Length = 148
pI of Protein: 10.7
Protein MW: 16044

TABLE XXVII

Accession EAW75952 hCG38472 [gi:119596358][[;]]
Span of LC/MS/MS identified peptides underlined:

(SEQ ID NO: 16)
MPSRLRKTQKLRGHVSHGHGRIGKLQKHPRGHSNAGGMHHHRINFNKYYP
GYFGKVGMRYYLKRNQTVSLDKLWTLVSEQTQVNAAKNKPGAAPLIDVVQ
SGYYKVLGKEKLPQPVIVKAKFFSRRAEKIKGVKGTCVLVA
gb|EAW75952.1] hCG38472 [Homo sapiens]
Length = 142
pI of Protein: 10.7
Protein MW: 16044

TABLE XXVIII
Accession Q9NQC3. [gi:17369290];
Span of LCIMSIMS identified peptides underlined:

(SEQ ID NO: 18)
MEDLDQSPLV SSSDSPPRPQ PAFKYQFVRE PEDEEEEEEE
EEEDEDEDLE ELEVLERKPA AGLSAAPVPT APAAGAPLMD
FGNDFVPPAP RGPLPAAPPV APERQPSWDP SPVSSTVPAP
SPLSAAAVSP SKLPEDDEPP ARPPPPPPAS VSPQAEPVWT
PPAPAPAAPP STPAAPKRRG SSGSVDETLF ALPAASEPVI
RSSAENMDLK EQPGNTISAG QEDFPSVLLE TAASLPSLSP
LSAASFKEHE YLGNLSTVLP TEGTLQENVS EASKEVSEKA
KTLLIDRDLT EFSELEYSEM GSSFSVSPKA ESAVIVANPR
EEIIVKNKDE EEKLVSNNIL HNQQELPTAL TKLVKEDEVV
SSEKAKDSFN EKRVAVEAPM REEYADFKPF ERVWEVKDSK
EDSDMLAAGG KIESNLESKV DKKCFADSLE QTNHEKDSES
SNDDTSFPST PEGIKDRSGA YITCAPFNPA ATESIATNIF
PLLGDPTSEN KTDEKKIEEK KAQIVTEKNT STKTSNPFLV
AAQDSETDYV TTDNLTKVTE EVVANMPEGL TPDLVQEACE
SELNEVTGTK IAYETKMDLV QTSEVMQESL YPAAQLCPSF
EESEATPSPV LPDIVMEAPL NSAVPSAGAS VIQPSSSPLE
ASSVNYESIK HEPENPPPYE EAMSVSLKKV SGIKEEIKEP
ENINAALQET EAPYISIACD LIKETKLSAE PAPDFSDYSE
MAKVEQPVPD HSELVEDSSP DSEPVDLFSD DSIPDVPQKQ
DETVMLVKES LTETSFESMI EYENKEKLSA LPPEGGKPYL
ESFKLSLDNT KDTLLPDEVS TLSKKEKIPL QMEELSTAVY
SNDDLFISKE AQIRETETFS DSSPIEIIDE FPTLISSKTD
SFSKLAREYT DLEVSHKSEI ANAPDGAGSL PCTELPHDLS
LKNIQPKVEE KISFSDDFSK NGSATSKVLL LPPDVSALAT
QAEIESIVKP KVLVKEAEKK LPSDTEKEDR SPSAIFSAEL
SKTSVVDLLY WRDIKKTGVV FGASLFLLLS LTVFSIVSVT
AYIALALLSV TISFRIYKGV IQAIQKSDEG HPFRAYLESE
VAISEELVQK YSNSALGHVN CTIKELRRLF LVDDLVDSLK
FAVLMWVFTY VGALFNGLTL LILALISLFS VPVIYERHQA
QIDHYLGLAN KNVKDAMAKI QAKIPGLKRK AE
pI of Protein: 4.4
Protein MW: 129932
Alternative names for B7108: (Neurite outgrowth inhibitor) (Nogo protein) (Foocen) (Neuroendocrine-specific protein) (NSP) (Neuroendocrine-specific protein C homolog) (RTN-x) (Reticulon-5)

TABLE XXIX
Span of LC/MS/MS identified peptides underlined:

(SEQ ID NO: 19)
DFTLFALPAA SEPVIRSSAE NMDLKEQPGN TISAGQEDFP
SVLLETAASL PSLSPLSAAS FKEHEYLGNL STVLPTEGTL
QENVSEASKE VSEKAKTLLI DRDLTEFSEL EYSEMGSSFS
VSPKAESAVI VANPR
(SEQ ID NO: 19)
pI of Protein: 4.3
Protein MW: 14420

TABLE XXX
Span of LC/MS/MS identified peptides underlined:

(SEQ ID NO: 20)
AESAVI VANPREEIIV KNKDEEEKLV SNNILHNQQE LPTALTKLVK
EDEVVSSEKA KDSFNEKRVA VEAPMREEYA DFKPFERVWE
VKDSKEDSDM LAAGGKIESN LESKVDKK
pI of Protein: 4.8
Protein MW: 12932

TABLE XXXI
Span of LC/MS/MS identified peptides underlined:

(SEQ ID NO: 21)
AESAVI VANPREEIIV KNKDEEEKLV SNNILHNQQE LPTALTKLVK
EDEVVSSEKA KDSFNEKRVA VEAPMREEYA DFKPFERVWE
VKDSKEDSDM LAAGGKIESN LESKVDKK CF ADSLEQTNHE
KDSESSNDDT SFPSTPEGIK DR
pI of Protein: 4.6
Protein MW: 16701

TABLE XXXII
Span of LC/MS/MS identified peptides underlined:

(SEQ ID NO: 22)
AESAVI VANPREEIIV KNKDEEEKLV SNNILHNQQE LPTALTKLVK
EDEVVSSEKA KDSFNEKRVA VEAPMREEYA DFKPFERVWE
VKDSKEDSDM LAAGGKIESN LESKVDKK CF ADSLEQTNHE K
pI of Protein: 4.8
Protein MW: 14435

TABLE XXXIII

| | | | | | |
|---|---|---|---|---|---|
| Gels | Patients | Mean | SE | Median | IQR |

a): B2422, down-regulated in breast cancer

| | | | | | | |
|---|---|---|---|---|---|---|
| ITI(H4) RP 35 KD Isoform | | | | | | |
| Protein Spot B2422 | | | | | | |
| Retrospective Samples | | | | | | |
| N | 192 | 64 | 45.8 | 3.63 | 32.0 | 39.3 |
| B9 | 344 | 115 | 45.8 | 2.61 | 32.0 | 64.1 |
| BC | 294 | 98 | 34.6 | 3.09 | 8.8 | 56.1 |

b): B2505, up-regulated in breast cancer*

| | | | | | | |
|---|---|---|---|---|---|---|
| ITI(H4) RP 35 KD Isoform | | | | | | |
| Protein Spot B2505 | | | | | | |
| Retrospective Samples | | | | | | |
| N | 192 | 64 | 104.3 | 4.39 | 95.5 | 54.0 |
| B9 | 344 | 115 | 101.7 | 3.10 | 88.2 | 74.9 |
| BC | 294 | 98 | 114.6 | 5.02 | 89.8 | 92.6 |

c): B3410, down-regulated in breast cancer

| | | | | | | |
|---|---|---|---|---|---|---|
| ITI(H4) RP 35 KD Isoform | | | | | | |
| Protein Spot B3410 | | | | | | |
| Retrospective Samples | | | | | | |
| N | 192 | 64 | 19.3 | 1.69 | 13.3 | 18.5 |
| B9 | 344 | 115 | 17.6 | 1.17 | 10.1 | 29.2 |
| BC | 294 | 98 | 14.6 | 1.41 | 0.0 | 26.6 |

d): B4404, down-regulated in breast cancer

| | | | | | | |
|---|---|---|---|---|---|---|
| ITI(H4) RP 35 KD Isoform | | | | | | |
| Protein Spot B4404 | | | | | | |
| Retrospective Samples | | | | | | |
| N | 192 | 64 | 21.2 | 1.43 | 17.0 | 19.4 |
| B9 | 344 | 115 | 23.1 | 1.41 | 16.9 | 16.5 |
| BC | 294 | 98 | 16.0 | 1.35 | 10.0 | 21.9 |

e) Sum of B2422 + B2505 + B3410 + B4404: “down-regulated” in breast cancer*

| | | | | | | |
|---|---|---|---|---|---|---|
| ITI(H4) RP 35 KD | | | | | | |
| PPM Sum of Isoforms | | | | | | |
| B2422 + B2505 + B3410 + B4404 | | | | | | |
| Retrospective Samples | | | | | | |
| N | 192 | 64 | 190.6 | 9.35 | 168.5 | 108.5 |
| B9 | 344 | 115 | 188.2 | 6.10 | 161.8 | 144.3 |
| BC | 294 | 98 | 179.9 | 9.46 | 117.4 | 137.4 |

*One of the isoforms that make up the sum, B2505 (b), is actually up-regulated. This is due to the lack of a significant down-regulation of B2505 in non-DCIS breast cancer patients (FIG. 4b; Table XXXVb). Thus, the up-regulation observed comes from the contribution from the more pronounced up regulation in the DCIS breast cancer patients within the breast cancer group.

TABLE XXXIV
Total ITI (H4) RP 35 KD Proteins = Sum of Protein Spots B2422 + B2505 + B3410 + B4404
Blood Serum Concentration Measured as 2D Gel Spot Density (PPM)
Retrospective vs. Prospective Samples
a) Concentration in 2D gel spot density:
Total ITI (H4) RP 35 KD Proteins = Sum of 2D gel spot density (PPM) of protein spots
B2422 + B2505 + B3410 + B4404

| | | Gels | Patients | Mean | SE | Median | IQR |
|---|---|---|---|---|---|---|---|
| | ITI(H4) RP 35 KD | | | | | | |
| | PPM Sum of Isoforms | | | | | | |
| | B2422 + B2505 + | | | | | | |
| | B3410 + B4404 | | | | | | |
| Retrospective | N | 192 | 64 | 190.6 | 9.35 | 168.5 | 108.5 |
| Samples | B9 | 344 | 115 | 188.2 | 6.10 | 161.8 | 144.3 |
| | N + B9 | 536 | 179 | 189.1 | 5.15 | 165.5 | 127.7 |
| | BC | 294 | 98 | 179.9 | 9.46 | 117.4 | 137.4 |
| Prospective | N | 48 | 16 | 282.2 | 21.96 | 273.0 | 163.4 |
| Samples | BC | 36 | 12 | 212.6 | 12.16 | 223.8 | 118.6 |
| | Total ITI (H4) RP 35 KD | | | | | | |
| | Isoform Spots = B2422 + | | | | | | |
| | B2505 + B2410 + B4404 | | | | | | |
| Retrospective | N | 240 | 80 | 209.74 | 15.11 | 177.29 | 108.23 |
| and Prospective | B9 | 327 | 109 | 188.00 | 10.46 | 165.76 | 148.48 |
| Combined | N + B9 | 567 | 189 | 197.20 | 8.80 | 171.88 | 136.39 |
| With and | Combined BC | 312 | 104 | 188.70 | 15.33 | 127.71 | 161.10 |
| Without DCIS | Non-DCIS BC | 222 | 74 | 148.96 | 13.51 | 106.10 | 117.82 |
| | DCIS BC | 90 | 30 | 286.72 | 36.00 | 218.14 | 291.90 |

b) Differential Expression in Fold of Average Normal Concentration;
Concentration = Fold of Average 2D Gel Spot Density (PPM)*
Total ITI(H4) RP 35 KD = Protein Spots B2422 + B2505 + B3410 + B4404

| | | Gels | Patients | Mean | SE | Median | Min | Max | IQR |
|---|---|---|---|---|---|---|---|---|---|
| | Retrospective | | | | | | | | |
| b1 | N | 192 | 64 | 1.000 | 0.049 | 0.884 | 0.199 | 5.714 | 0.569 |
| | B9 | 344 | 115 | 0.988 | 0.032 | 0.849 | 0.148 | 3.292 | 0.757 |
| | BC | 294 | 98 | 0.944 | 0.050 | 0.616 | 0.020 | 4.933 | 0.721 |
| | Prospective | | | | | | | | |
| b2 | N | 51 | 17 | 1.000 | 0.075 | 0.931 | 0.214 | 2.628 | 0.534 |
| | BC | 39 | 13 | 0.775 | 0.041 | 0.848 | 0.277 | 1.203 | 0.392 |
| | Combined +/− DCIS | | | | | | | | |
| b3 | N + B9 | 567 | 189 | 1.003 | 0.044 | 0.876 | 0.211 | 4.965 | 0.664 |
| | Non-DCIS BC | 234 | 78 | 0.747 | 0.085 | 0.538 | 0.048 | 3.624 | 0.601 |
| | DCIS BC | 90 | 30 | 1.482 | 0.191 | 1.130 | 0.231 | 4.548 | 1.566 |

*Determined separately for prospective and retrospective samples, then combined in b3

TABLE XXXV
Fold of Average Normal PPM or μg protein/ml of blood serum
a.

| ITI (H4) RP 35 KD | | | | | | |
|---|---|---|---|---|---|---|
| Isoform Spot B2422 | Gels | Patients | Mean | SE | Median | IQR |
| N | 240 | 80 | 1.008 | 0.110 | 0.812 | 0.828 |
| B9 | 327 | 109 | 1.011 | 0.099 | 0.758 | 1.452 |
| N + B9 | 567 | 189 | 1.009 | 0.073 | 0.803 | 1.144 |
| Combined BC | 312 | 104 | 0.799 | 0.108 | 0.312 | 1.241 |
| Non-DCIS BC | 222 | 74 | 0.567 | 0.105 | 0.181 | 0.800 |
| DCIS BC | 90 | 30 | 1.372 | 0.242 | 1.131 | 2.016 |

b.

| ITI (H4) RP 35 KD | | | | | | |
|---|---|---|---|---|---|---|
| Isoform Spot B2505 | Gels | Patients | Mean* | SE | Median | IQR |
| N | 240 | 80 | 1.001 | 0.059 | 0.944 | 0.481 |
| B9 | 327 | 109 | 0.968 | 0.050 | 0.850 | 0.687 |
| N + B9 | 567 | 189 | 0.982 | 0.038 | 0.888 | 0.590 |
| Combined BC | 312 | 104 | 1.102 | 0.077 | 0.841 | 0.820 |
| Non-DCIS BC | 222 | 74 | 0.915 | 0.063 | 0.758 | 0.684 |
| DCIS BC | 90 | 30 | 1.564 | 0.196 | 1.161 | 1.166 |

c.

| ITI (H4) RP 35 KD | | | | | | |
|---|---|---|---|---|---|---|
| Isoform Spot B3410 | Gels | Patients | Mean | SE | Median | IQR |
| N | 240 | 80 | 1.006 | 0.117 | 0.803 | 0.919 |
| B9 | 327 | 109 | 0.920 | 0.103 | 0.588 | 1.591 |
| N + B9 | 567 | 189 | 0.957 | 0.077 | 0.752 | 1.487 |
| Combined BC | 312 | 104 | 0.806 | 0.116 | 0.229 | 1.353 |
| Non-DCIS BC | 222 | 74 | 0.535 | 0.102 | 0.000 | 0.731 |
| DCIS BC | 90 | 30 | 1.474 | 0.281 | 1.226 | 2.383 |

d.

| ITI (H4) RP 35 KD | | | | | | |
|---|---|---|---|---|---|---|
| Isoform Spot B4404 | Gels | Patients | Mean | SE | Median | IQR |
| N | 240 | 80 | 1.004 | 0.130 | 0.809 | 0.734 |
| B9 | 327 | 109 | 1.084 | 0.108 | 0.847 | 0.675 |
| N + B9 | 567 | 189 | 1.050 | 0.083 | 0.824 | 0.719 |
| Combined BC | 312 | 104 | 0.727 | 0.091 | 0.482 | 0.901 |
| Non-DCIS BC | 222 | 74 | 0.548 | 0.096 | 0.350 | 0.735 |
| DCIS BC | 90 | 30 | 1.170 | 0.189 | 0.860 | 1.306 |

*Insignificant down-regulation of b. B2505 in non-DCIS breast cancer patients, as compared To a. B2422, c. B3410, and d. B4404.

TABLE XXXVI

| Immunoglobulin | | | | | | |
|---|---|---|---|---|---|---|
| Lambda | | | | | | |
| Chain Protein | | | | | | |
| Spot B1322 | Gels | Patients | Mean | SE | Median | IQR |
| N | 240 | 80 | 1.004 | 0.054 | 0.911 | 0.572 |
| B9 | 327 | 109 | 0.915 | 0.039 | 0.816 | 0.447 |
| N + B9 | 567 | 189 | 0.953 | 0.032 | 0.852 | 0.477 |
| Combined BC | 312 | 104 | 0.931 | 0.043 | 0.841 | 0.483 |
| Non-DCIS BC | 222 | 74 | 0.916 | 0.049 | 0.818 | 0.506 |
| DCIS BC | 90 | 30 | 0.966 | 0.086 | 0.898 | 0.344 |

TABLE XXXVII

| Alpha-1-microglobulin | | | | | | |
|---|---|---|---|---|---|---|
| Protein Spot B1418 | Gels | Patients | Mean | SE | Median | IQR |
| N | 240 | 80 | 1.000 | 0.035 | 0.934 | 0.427 |
| B9 | 327 | 109 | 1.092 | 0.046 | 0.956 | 0.619 |
| N + B9 | 567 | 189 | 1.053 | 0.031 | 0.944 | 0.557 |
| Combined BC | 312 | 104 | 1.212 | 0.069 | 1.053 | 0.522 |
| Non-DCIS BC | 222 | 74 | 1.192 | 0.088 | 1.009 | 0.568 |
| DCIS BC | 90 | 30 | 1.259 | 0.102 | 1.168 | 0.361 |

TABLE XXXVIII

| Apolipoprotein A1 | | | | | | |
|---|---|---|---|---|---|---|
| Protein B2317 | Gels | Patients | Mean | SE | Median | IQR |
| N | 240 | 80 | 0.996 | 0.037 | 0.984 | 0.378 |
| B9 | 327 | 109 | 0.842 | 0.033 | 0.794 | 0.516 |
| N + B9 | 567 | 189 | 0.907 | 0.025 | 0.904 | 0.445 |
| Combined BC | 312 | 104 | 1.095 | 0.071 | 0.943 | 0.550 |
| Non-DCIS BC | 222 | 74 | 0.950 | 0.051 | 0.874 | 0.478 |
| DCIS BC | 90 | 30 | 1.453 | 0.198 | 1.242 | 0.497 |

TABLE XXXIX

| Apolipoprotein E3 | | | | | | |
|---|---|---|---|---|---|---|
| Protein Spot B3406 | Gels | Patients | Mean | SE | Median | IQR |
| N | 240 | 80 | 0.988 | 0.066 | 0.827 | 0.725 |
| B9 | 327 | 109 | 0.970 | 0.069 | 0.871 | 0.918 |
| N + B9 | 567 | 189 | 0.977 | 0.049 | 0.860 | 0.835 |
| Combined BC | 312 | 104 | 1.023 | 0.070 | 0.856 | 0.753 |
| Non-DCIS BC | 222 | 74 | 0.947 | 0.071 | 0.825 | 0.695 |
| DCIS BC | 90 | 30 | 1.211 | 0.164 | 0.948 | 0.878 |

TABLE XL

| Serum Albumin | | | | | | |
|---|---|---|---|---|---|---|
| Protein Spot B5539 | Gels | Patients | Mean | SE | Median | IQR |
| N | 240 | 80 | 1.001 | 0.093 | 0.948 | 0.342 |
| B9 | 327 | 109 | 1.170 | 0.032 | 1.139 | 0.404 |
| N + B9 | 567 | 189 | 1.098 | 0.044 | 1.017 | 0.355 |
| Combined BC | 312 | 104 | 0.896 | 0.034 | 0.892 | 0.465 |
| Non-DCIS BC | 222 | 74 | 0.854 | 0.034 | 0.856 | 0.379 |
| DCIS BC | 90 | 30 | 0.999 | 0.081 | 1.081 | 0.599 |

TABLE XLI

| Transferrin Protein | | | | | | |
|---|---|---|---|---|---|---|
| Spot B6605 | Gels | Patients | Mean | SE | Median | IQR |
| N | 240 | 80 | 1.003 | 0.039 | 0.926 | 0.406 |
| B9 | 327 | 109 | 1.186 | 0.046 | 1.151 | 0.537 |
| N + B9 | 567 | 189 | 1.109 | 0.032 | 1.045 | 0.506 |
| Combined BC | 312 | 104 | 1.107 | 0.055 | 1.034 | 0.615 |
| Non-DCIS BC | 222 | 74 | 1.086 | 0.062 | 0.993 | 0.597 |
| DCIS BC | 90 | 30 | 1.157 | 0.116 | 1.167 | 0.681 |

TABLE XLII

| Serotransferin Protein | | | | | | |
|---|---|---|---|---|---|---|
| Spot B5713 | Gels | Patients | Mean | SE | Median | IQR |
| N | 240 | 80 | 0.992 | 0.081 | 0.866 | 0.723 |
| B9 | 327 | 109 | 0.856 | 0.059 | 0.682 | 0.771 |
| N + B9 | 567 | 189 | 0.914 | 0.048 | 0.747 | 0.737 |
| Combined BC | 312 | 104 | 0.833 | 0.066 | 0.612 | 0.790 |
| Non-DCIS BC | 222 | 74 | 0.841 | 0.084 | 0.587 | 0.841 |
| DCIS BC | 90 | 30 | 0.816 | 0.099 | 0.652 | 0.578 |

TABLE XLIII

| Haptoglobin Protein | | | | | | |
|---|---|---|---|---|---|---|
| Spot B1512 | Gels | Patients | Mean | SE | Median | IQR |
| N | 240 | 80 | 0.995 | 0.058 | 0.957 | 0.814 |
| B9 | 327 | 109 | 1.206 | 0.060 | 1.128 | 0.836 |
| N + B9 | 567 | 189 | 1.116 | 0.043 | 1.063 | 0.817 |
| Combined BC | 312 | 104 | 1.418 | 0.068 | 1.354 | 0.865 |
| Non-DCIS BC | 222 | 74 | 1.483 | 0.083 | 1.426 | 0.897 |
| DCIS BC | 90 | 30 | 1.259 | 0.115 | 1.125 | 0.954 |

TABLE XLIV

| Haptoglobin Protein | | | | | | |
|---|---|---|---|---|---|---|
| Spot B6014 | Gels | Patients | Mean | SE | Median | IQR |
| N | 240 | 80 | 1.013 | 0.198 | 0.000 | 1.345 |
| B9 | 327 | 109 | 0.749 | 0.155 | 0.000 | 0.131 |
| N + B9 | 567 | 189 | 0.860 | 0.122 | 0.000 | 1.061 |
| Combined BC | 312 | 104 | 1.821 | 0.319 | 0.085 | 2.784 |
| Non-DCIS BC | 222 | 74 | 2.091 | 0.405 | 0.322 | 3.066 |
| DCIS BC | 90 | 30 | 1.154 | 0.455 | 0.000 | 2.086 |

TABLE XLV

| Haptoglobin Protein | | | | | | |
|---|---|---|---|---|---|---|
| Spot B4008 | Gels | Patients | Mean | SE | Median | IQR |
| N | 240 | 80 | 0.977 | 0.086 | 0.855 | 1.054 |
| B9 | 327 | 109 | 1.277 | 0.130 | 1.019 | 1.329 |
| N + B9 | 567 | 189 | 1.150 | 0.084 | 0.933 | 1.231 |
| Combined BC | 312 | 104 | 1.311 | 0.139 | 0.976 | 0.947 |
| Non-DCIS BC | 222 | 74 | 1.210 | 0.100 | 0.994 | 0.942 |
| DCIS BC | 90 | 30 | 1.561 | 0.417 | 0.859 | 0.962 |

TABLE XLVI

| Haptoglobin Protein | | | | | | |
|---|---|---|---|---|---|---|
| Spot B4206 | Gels | Patients | Mean | SE | Median | IQR |
| N | 240 | 80 | 0.975 | 0.110 | 0.864 | 1.196 |
| B9 | 327 | 109 | 1.394 | 0.134 | 1.133 | 1.308 |
| N + B9 | 567 | 189 | 1.217 | 0.091 | 0.982 | 1.352 |
| Combined BC | 312 | 104 | 1.579 | 0.167 | 1.274 | 1.880 |
| Non-DCIS BC | 222 | 74 | 1.390 | 0.167 | 1.094 | 2.290 |
| DCIS BC | 90 | 30 | 2.045 | 0.396 | 1.654 | 1.230 |

TABLE XLVII

| Haptoglobin Related | | | | | | |
|---|---|---|---|---|---|---|
| Protein Spot B3506 | Gels | Patients | Mean | SE | Median | IQR |
| N | 240 | 80 | 1.013 | 0.099 | 0.762 | 1.383 |
| B9 | 327 | 109 | 1.002 | 0.187 | 0.706 | 1.214 |
| N + B9 | 567 | 189 | 1.006 | 0.115 | 0.710 | 1.294 |
| Combined BC | 312 | 104 | 0.940 | 0.094 | 0.701 | 1.443 |
| Non-DCIS BC | 222 | 74 | 0.960 | 0.115 | 0.847 | 1.514 |
| DCIS BC | 90 | 30 | 0.892 | 0.162 | 0.638 | 1.122 |

TABLE XLVIII

| Haptoglobin Related | | | | | | |
|---|---|---|---|---|---|---|
| Protein Spot B4424 | Gels | Patients | Mean | SE | Median | IQR |
| N | 240 | 80 | 0.999 | 0.104 | 0.800 | 1.166 |
| B9 | 327 | 109 | 1.045 | 0.080 | 0.955 | 0.945 |
| N + B9 | 567 | 189 | 1.025 | 0.063 | 0.918 | 1.077 |
| Combined BC | 312 | 104 | 0.930 | 0.069 | 0.893 | 0.816 |
| Non-DCIS BC | 222 | 74 | 0.953 | 0.087 | 0.887 | 0.813 |
| DCIS BC | 90 | 30 | 0.875 | 0.109 | 0.895 | 0.829 |

TABLE XLIX

| Lectin P35 3 Protein | | | | | | |
|---|---|---|---|---|---|---|
| Spot B6519 | Gels | Patients | Mean | SE | Median | IQR |
| N | 240 | 80 | 0.995 | 0.041 | 0.986 | 0.478 |
| B9 | 327 | 109 | 1.269 | 0.167 | 0.992 | 0.572 |
| N + B9 | 567 | 189 | 1.153 | 0.098 | 0.992 | 0.522 |
| Combined BC | 309 | 103 | 1.214 | 0.135 | 1.030 | 0.558 |
| Non-DCIS BC | 222 | 74 | 1.143 | 0.111 | 1.038 | 0.531 |
| DCIS BC | 87 | 29 | 1.393 | 0.391 | 0.977 | 0.605 |

TABLE L

| Complement | | | | | | |
|---|---|---|---|---|---|---|
| C4A gamma | | | | | | |
| Protein Spot B7408 | Gels | Patients | Mean | SE | Median | IQR |
| N | 240 | 80 | 1.008 | 0.069 | 0.862 | 0.863 |
| B9 | 327 | 109 | 1.273 | 0.077 | 1.058 | 0.918 |
| N + B9 | 567 | 189 | 1.161 | 0.054 | 0.992 | 0.903 |
| Combined BC | 312 | 104 | 1.320 | 0.104 | 1.062 | 0.864 |
| Non-DCIS BC | 222 | 74 | 1.180 | 0.106 | 0.992 | 0.830 |
| DCIS BC | 90 | 30 | 1.664 | 0.238 | 1.177 | 1.440 |

TABLE LI

| Apoptosis | | | | | | |
|---|---|---|---|---|---|---|
| Inhibitor (CD5L) | | | | | | |
| Protein Spot B2412 | Gels | Patients | Mean | SE | Median | IQR |
| N | 240 | 80 | 1.002 | 0.031 | 0.989 | 0.329 |
| B9 | 327 | 109 | 1.052 | 0.036 | 0.938 | 0.431 |
| N + B9 | 567 | 189 | 1.031 | 0.025 | 0.967 | 0.361 |
| Combined BC | 312 | 104 | 1.181 | 0.058 | 1.056 | 0.521 |
| Non-DCIS BC | 222 | 74 | 1.154 | 0.070 | 1.046 | 0.556 |
| DCIS BC | 90 | 30 | 1.250 | 0.101 | 1.093 | 0.389 |

TABLE LII

| Nucleolar | | | | | | |
|---|---|---|---|---|---|---|
| Ribosomal Protein | | | | | | |
| L27a Spot B6218 | Gels | Patients | Mean | SE | Median | IQR |
| N | 240 | 80 | 0.909 | 0.114 | 0.672 | 1.217 |
| B9 | 327 | 109 | 0.835 | 0.089 | 0.539 | 1.106 |
| N + B9 | 567 | 189 | 0.866 | 0.070 | 0.604 | 1.147 |
| Combined BC | 312 | 104 | 1.514 | 0.170 | 1.010 | 1.873 |
| Non-DCIS BC | 222 | 74 | 1.383 | 0.193 | 0.989 | 1.905 |
| DCIS BC | 90 | 30 | 1.838 | 0.346 | 1.143 | 2.017 |

TABLE LIII

| Neuroendocrie Specific | | | | | | |
|---|---|---|---|---|---|---|
| (NSP) Protein | | | | | | |
| Spot B7108 | Gels | Patients | Mean | SE | Median | IQR |
| N | 240 | 80 | 1.003 | 0.051 | 0.908 | 0.571 |
| B9 | 327 | 109 | 0.844 | 0.050 | 0.768 | 0.474 |
| N + B9 | 567 | 189 | 0.911 | 0.036 | 0.816 | 0.516 |
| Combined BC | 312 | 104 | 0.748 | 0.047 | 0.717 | 0.640 |
| Non-DCIS BC | 222 | 74 | 0.722 | 0.061 | 0.630 | 0.722 |
| DCIS BC | 90 | 30 | 0.812 | 0.066 | 0.746 | 0.467 |

TABLE LIV

| Number of Observations and | | | | Number of Observations | | |
|---|---|---|---|---|---|---|
| Percent Classified into Diagnosis | | | | and Percent Classified | | |
| Step Disk 9 Biomarkers | | | | All 22 Biomarkers | | |
| From | Control | Combined | | From | Control | Combined |
| Diagnosis | (N + B9) | BC | Total | Diagnosis | (N + B9) | BC |
| N + B9 | 141 | 48 | 189 | N + B9 | 143 | 46 |
| | 74.60% | 25.40% | | | 75.66% | 24.34% |
| DCIS BC | 6 | 24 | 30 | DCIS BC | 5 | 25 |
| | 20.00% | 80.00% | | | 16.67% | 83.33% |
| Non-DCIS BC | 19 | 55 | 74 | Non-DCIS BC | 19 | 55 |
| | 25.68% | 74.32% | | | 25.68% | 74.32% |
| Combined BC | 24 | 80 | 104 | Combined BC | 24 | 80 |
| | 23.08% | 76.92% | | | 23.08% | 76.92% |

TABLE LV

| | Normal | B9 | DCIS BC | Non-DCIS BC |
|---|---|---|---|---|
| | Median | Median | Median | Median |
| B2317 | 0.984 | 0.794 | 1.242 | 0.874 |
| B2505 | 0.944 | 0.850 | 1.161 | 0.758 |
| B6218 | 0.672 | 0.539 | 1.143 | 0.989 |
| B6014 | 0.000 | 0.000 | 0.000 | 0.322 |
| B1512 | 0.957 | 1.128 | 1.125 | 1.426 |
| B7108 | 0.908 | 0.768 | 0.746 | 0.630 |
| B5539 | 0.948 | 1.139 | 1.081 | 0.856 |
| B2422 | 0.812 | 0.758 | 1.131 | 0.181 |
| B4404 | 0.809 | 0.847 | 0.860 | 0.350 |
| B3410 | 0.803 | 0.588 | 1.226 | 0.000 |
| B7408 | 0.862 | 1.058 | 1.177 | 0.992 |
| B4008 | 0.855 | 1.019 | 0.859 | 0.994 |
| B4206 | 0.864 | 1.133 | 1.654 | 1.094 |
| B2412 | 0.989 | 0.938 | 1.093 | 1.046 |
| B1322 | 0.911 | 0.816 | 0.898 | 0.818 |
| B1418 | 0.934 | 0.956 | 1.168 | 1.009 |
| B3406 | 0.827 | 0.871 | 0.948 | 0.825 |
| B6519 | 0.986 | 0.992 | 1.055 | 1.038 |
| B6605 | 0.926 | 1.151 | 1.167 | 0.993 |
| B3506 | 0.762 | 0.706 | 0.638 | 0.847 |
| B4424 | 0.800 | 0.955 | 0.895 | 0.887 |
| B5713 | 0.866 | 0.682 | 0.652 | 0.587 |

### REFERENCES

1. Tan, P. K. et al. Evaluation of gene expression measurements from commercial microarray platforms. Nucleic Acids Res 31, 5676-5684 (2003).
2. Marshall, E. Getting the noise out of gene arrays. Science 306, 630-631 (2004).
3. Shi, L. et al. The MicroArray Quality Control (MAQC) project shows inter- and intraplatform reproducibility of gene expression measurements. Nat Biotechnol 24, 1151-1161 (2006).
4. Guo, L. et al. Rat toxicogenomic study reveals analytical consistency across microarray platforms. Nat Biotechnol 24, 1162-1169 (2006).
5. Canales, R. D. et al. Evaluation of DNA microarray results with quantitative gene expression platforms. Nat Biotechnol 24, in press (2006).
6. Shippy, R. et al. Using RNA sample titrations to assess microarray platform performance and normalization techniques. Nat Biotechnol 24, 1123-1131 (2006).
7. Patterson, T. A. et al. Performance comparison of one-color and two-color platforms within the Microarray Quality Control (MAQC) project. Nat Biotechnol 24, 1140-1150 (2006).
8. Tong, W. et al. Evaluation of external RNA controls for the assessment of microarray performance. Nat Biotechnol 24, 1132-1139 (2006).
9. Casciano, D. A. & Woodcock, J. Empowering microarrays in the regulatory setting. Nat Biotechnol 24, 1103 (2006).
10. Making the most of microarrays. Nat Biotechnol 24, 1039 (2006).
11. Frueh, F. W. Impact of microarray data quality on genomic data submissions to the FDA. Nat Biotechnol 24, 1105-1107 (2006).
12. Dix, D. J. et al. A framework for the use of genomics data at the EPA. Nat Biotechnol 24, 1108-1111 (2006).
13. Ji, H. & Davis, R. W. Data quality in genomics and microarrays. Nat Biotechnol 24, 1112-1113 (2006).
14. Reid, L. H. & Warrington, J. A. A note on nomenclature. Nat Biotechnol 24, ii (2006).
15. Strauss, E. Arrays of hope. Cell 127, 657-659 (2006).
16. Eisenstein, M. Microarrays: quality control. Nature 442, 1067-1070 (2006).
17. Couzin, J. Genomics. Microarray data reproduced, but some concerns remain. Science 313, 1559 (2006).
18. Kiermer, V. Microarray quality in the spotlight again. Nat Methods 3, 772 (2006).
19. Sage, L. Do microarrays measure up? Anal Chem 78, 7358-7360 (2006).
20. Michiels, S., Koscielny, S. & Hill, C. Prediction of cancer outcome with microarrays: a multiple random validation strategy. Lancet 365, 488-492 (2005).
21. Ioannidis, J. P. Microarrays and molecular research: noise discovery? Lancet 365, 454-455 (2005).
22. Ein-Dor, L., Zuk, O. & Domany, E. Thousands of samples are needed to generate a robust gene list for predicting outcome in cancer. Proc Natl Acad Sci USA 103, 5923-5928 (2006).
23. Simon, R. Development and evaluation of therapeutically relevant predictive classifiers using gene expression profiling. J Natl Cancer Inst 98, 1169-1171 (2006).

What is claimed is:

1. Twenty two (22) protein biomarkers as related to breast cancer.

2. A method for screening, diagnosis, or staging of patients with breast cancer, whereby 1, 2, or more of up to the 22 protein biomarkers of claim 1 in human blood identified as related to breast cancer are employed for differentiating between patients having an earlier and/or later stage of breast cancer, patients having a benign breast disease or abnormality, and normal control individuals. The method comprises:
collecting a whole blood, blood serum, or blood plasma sample from a test subject;
determining the concentrations of up to 22 protein biomarkers identified as related to breast cancer in the test subject sample, and
determining the concentrations of up to 22 protein biomarkers identified as related to breast cancer in samples from patients having biopsy confirmed and histological staged breast cancer, patients having a benign breast abnormality or benign breast disease, and normal control individuals having no evidence of breast disease or breast abnormality,
Performing a statistical analysis and determining whether or not the test subject is normal, has benign breast disease or abnormality or has an earlier and/or later stage of breast cancer, based on a statistical analysis of the concentration in blood serum of the one, two or more of the selected 22 protein biomarkers.

3. The method of claim 2, wherein the concentration of the protein biomarkers are determined by first separating the proteins by 2D gel electrophoresis.

4. The method of claim 2, wherein the statistical analysis is an analysis of variance, a multivariate linear or quadratic discriminant analysis, a multivariate canonical discriminant analysis, a receiver operator characteristics (ROC) analysis, and/or a statistical plot such as a Box and Whiskers plot and/or a receiver operator characteristics (ROC) plot.

5. One, two or more biomarkers of claim 1, wherein the biomarker is one, two or more of the following 22 biomarkers:
An inter-alpha-trypsin inhibitor heavy chain (H4) related protein and/or one or more of the biomarker protein isoforms, and/or post-synthetic modification variants, and/or processing products thereof, and/or
an immunoglobulin lambda chain protein, and/or one or more of the biomarker protein isoforms, and/or post-synthetic modification variants, and/or processing products thereof, and/or
an alpha-1-microglobulin protein, and/or one or more of the biomarker protein isoforms, and/or post-synthetic modification variants, and/or processing products thereof, and/or
an Apolipoprotein A-I protein, and/or one or more of the biomarker protein isoforms, and/or post-synthetic modification variants, and/or processing products thereof, and/or
an Apolipoprotein E protein, an Apolipoprotein E3 protein, and/or one or more of the biomarker protein isoforms, and/or post-synthetic modification variants, and/or processing products thereof, and/or
a Complement C4 protein, a Complement C4A protein, a Complement C4A gamma chain protein, and/or one or more of the biomarker protein isoforms, and/or post-synthetic modification variants, and/or processing products thereof, and/or
a Serum Albumin protein, and/or one or more of the biomarker protein isoforms, and/or post-synthetic modification variants, and/or processing products thereof, and/or
a Lectin P35 protein, and/or one or more of the biomarker protein isoforms, and/or post-synthetic modification variants, and/or processing products thereof, and/or
a Transferrin protein, and/or one or more of the biomarker protein isoforms, and/or post-synthetic modification variants, and/or processing products thereof, and/or
a Haptoglobin protein, and/or one or more of the biomarker protein isoforms or post-synthetic modification variants of a Haptoglobin protein, and/or a processing product thereof, and/or
an Apoptosis inhibitor expressed by Macrophages (AIM), and/or a Human secreted protein CD5L, and/or one or more of the biomarker protein isoforms, and/or post-synthetic modification variants, and/or processing products thereof, and/or
a Haptoglobin-related protein, and/or one or more of the biomarker protein isoforms, and/or post-synthetic modification variants post-synthetic modification variants, and/or processing products thereof, and/or
a Serotransferrin protein and/or a Siderophilin protein and/or a Beta-1-metal-binding globulin protein, and/or one or more of the biomarker protein isoforms, and/or post-synthetic modification variants, and/or processing products thereof, and/or
a nucleolar protein, and/or a ribosomal protein, and/or a 60S ribosomal protein L27a protein, and/or one or more of the biomarker protein isoforms, and/or post-synthetic modification variants, and/or processing products thereof, and/or
a Reticulon-4 (Neurite outgrowth inhibitor) (Nogo protein) (Foocen) (Neuroendocrine-specific protein) (NSP) (Neuroendocrine-specific protein C homolog) (RTN-x) (Reticulon-5) protein, and/or one or more of the biomarker protein isoforms, and/or post-synthetic modification variants, and/or processing products thereof, and/or
one or more of the proteins comprising the amino acid sequences #1-23, referred to in Table VI, and/or depicted as protein spots: B1512; B1418; B1322; B2412; B2505: B3406; B2422; B3410; B3506; B4008; B4206; B4404; B4424; B5539; B5713; B6605; B6519; B6218; B6014; B7408; and/or B7108, in the 2D gels in FIG. 1 and/or FIG. 6.
